(19) United States
(12) Patent Application Publication
(10) Pub. No.: US 2022/0313822 A1
(43) Pub. Date: Oct. 6, 2022

(54) NEAR-INFRARED (NIR) PHOTOIMMUNOTHERAPY (PIT) FOR THE TREATMENT OF CANCERS USING ANTI-CD25 ANTIBODY-PHTHALOCYANINE DYE CONJUGATE AND ANTI-PD1 ANTIBODY

(71) Applicant: Rakuten Medical, Inc., San Mateo, CA (US)

(72) Inventors: Miguel GARCIA-GUZMAN, San Diego, CA (US); Roger HEIM, Del Mar, CA (US); Jerry FONG, San Mateo, CA (US)

(73) Assignee: Rakuten Medical, Inc., San Mateo, CA (US)

(21) Appl. No.: 17/630,854

(22) PCT Filed: Jul. 29, 2020
(86) PCT No.: PCT/US2020/043999

§ 371 Date: Jan. 27, 2022

Related U.S. Application Data
(60) Provisional application No. 62/931,405, filed on Nov. 6, 2019.
(60) Provisional application No. 62/896,453, filed on Sep. 5, 2019.
(60) Provisional application No. 62/880,514, filed on Jul. 30, 2019.

(51) Int. Cl. A61K 41/00; A61K 39/395; A61K 47/68; A61P 35/00

(52) U.S. Cl. CPC A61K 41/0071; A61K 39/3955; A61K 47/6849; A61P 35/00; A61K 2039/545

(57) ABSTRACT

The present disclosure relates to near-infrared (NIR) photoimmunotherapy (PIT) for treating a subject having a cancer, such as a cancer comprising a first tumor or a primary tumor, metastatic tumor cells and/or invasive tumor cells. The method includes administering to the subject a targeting molecule that binds CD25 conjugated with phthalocyanine dye, such as IR700, and administering an immune checkpoint inhibitor such as an anti PD-1 antibody, followed by illuminating the first tumor or primary tumor with a wavelength of light suitable for the activation of the phthalocyanine dye.

### RELATED APPLICATIONS

This application claims priority from U.S. Provisional Application No. 62/880,514, filed Jul. 30, 2019, entitled “COMPOSITIONS AND METHODS FOR LOCAL AND SYSTEMIC TREATMENT OF CANCERS, TUMORS AND TUMOR CELLS,” from U.S. Provisional Application No. 62/896,453, filed Sep. 5, 2019, entitled “COMPOSITIONS AND METHODS FOR LOCAL AND SYSTEMIC TREATMENT OF CANCERS, TUMORS AND TUMOR CELLS,” and from U.S. Provisional Application No. 62/931,405, filed Nov. 6, 2019, entitled “COMPOSITIONS AND METHODS FOR LOCAL AND SYSTEMIC TREATMENT OF CANCERS, TUMORS AND TUMOR CELLS,” the contents of which are incorporated by reference in their entirety.

### FIELD

The present disclosure relates to compositions, combinations, methods and uses for treating a subject having a cancer, such as a cancer comprising a first tumor or a primary tumor, metastatic tumor cells, and/or invasive tumor cells. The methods include administering to the subject a targeting molecule that binds CD25 conjugated with phthalocyanine dye, such as IR700, and administering an immune checkpoint inhibitor, followed by illuminating the first tumor or primary tumor with a wavelength of light suitable for the activation of the phthalocyanine dye. The methods and uses described herein provide for reduction, growth and elimination of tumors and tumor cells including first tumors, primary tumors, metastatic tumor cells, and/or invasive tumor cells. Also provided are compositions, combinations, methods and uses for enhancing systemic immunity against tumor growth in a subject having a cancer, such as a cancer comprising a first tumor or a primary tumor, metastatic tumor cells, and/or invasive tumor cells.

### BACKGROUND

Cancer metastasis is the primary cause of cancer-related deaths. Despite some available treatments for certain types of cancers, therapeutic strategies are still urgently needed for effectively treating cancers, including those that involve both primary and metastatic tumors. Treating metastatic cancers, in particular, still represents a great clinical challenge.

### SUMMARY

Provided herein are compositions, combinations, methods and uses for treating cancer, including cancers comprising a tumor, such as a first tumor, as well as those that have a secondary population of tumor cells, such as metastatic tumor cells, invasive tumor cells or infiltrating cells.

Provided herein are methods and uses for treating a cancer. In some of any embodiments, the provided methods and uses involve: administering an immune checkpoint inhibitor to a subject having a cancer comprising a first tumor and a secondary population of tumor cells; administering a conjugate comprising a phthalocyanine dye linked to an anti-CD25 antibody to the subject; and after administering the conjugate, illuminating the first tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the secondary population is not directly illuminated.

In some of any embodiments, the first tumor and/or the secondary population is inhibited to a greater degree as compared with administration of only the conjugate, only the conjugate followed by illumination, or only the immune checkpoint inhibitor. In some of any embodiments, the immune checkpoint inhibitor is selected from the group consisting of a PD-1 inhibitor, a PD-L1 inhibitor or a CTLA-4 inhibitor. In some of any embodiments, the immune checkpoint inhibitor is or comprises an anti-PD-1 antibody, an anti-PD-L1 antibody or an anti-CTLA-4 antibody, or an antigen-binding fragment thereof. In some of any embodiments, the immune checkpoint inhibitor is or comprises an anti-PD-1 antibody or an antigen-binding fragment thereof.

Provided herein are methods and uses for treating a cancer. In some of any embodiments, the provided methods and uses involve: administering an anti-PD-1 antibody to a subject having a cancer comprising a first tumor and a secondary population of tumor cells; administering a conjugate comprising a phthalocyanine dye linked to an anti-CD25 antibody to the subject; and after administering the conjugate, illuminating the first tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the secondary population is not directly illuminated; wherein the first tumor and/or the secondary population is inhibited to a greater degree as compared with administration of only the conjugate, only the conjugate followed by illumination, or only the anti-PD-1 antibody.

In some of any embodiments, inhibition comprises one or more of: less than at or about 20% increase in tumor volume, tumor dimensions or tumor mass, or a reduction in tumor volume, tumor dimensions or tumor mass, or a reduction in the number of tumor cells. In some of any embodiments, the reduction in tumor volume, tumor dimensions or tumor mass, or the number of tumor cells comprises at or about a 30% reduction or more.

In some of any embodiments, the secondary population comprises metastatic tumor cells. In some of any embodiments, the secondary population comprises invasive tumor cells. In some of any embodiments, the secondary population comprises metastatic tumor cells and invasive tumor cells.

In some of any embodiments, the immune checkpoint inhibitor is administered to the subject concurrently with the conjugate. In some of any embodiments, the immune checkpoint inhibitor is administered to the subject within 24 to 48 hours of administering the conjugate. In some of any embodiments, the immune checkpoint inhibitor is administered to the subject within 24 hours±4 hours of administering the conjugate.

In some of any embodiments, a first dose of the immune checkpoint inhibitor is administered prior to the administration of the conjugate. In some of any embodiments, the first dose of the immune checkpoint inhibitor is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

In some of any embodiments, the immune checkpoint inhibitor is administered once, twice, three times, four times, five times, six times, seven times, eight times, nine times, 10 times, or more than 10 times to the subject. In some of any embodiments, immune checkpoint inhibitor is administered once, twice, three times, four times, five times, six times, seven times, eight times, nine times, or 10 times before the administration of the conjugate, and subsequently followed by the administration of the immune checkpoint inhibitor zero times, once, twice, three times, four times, five times, six times, seven times, eight times, nine times, 10 times, or more than 10 times to the subject after administration of the conjugate. In some of any embodiments, the immune checkpoint inhibitor is administered concurrently with the administration of the conjugate, and subsequently followed by the administration of the immune checkpoint inhibitor zero times, once, twice, twice, three times, four times, five times, six times, seven times, eight times, nine times, 10 times, or more than 10 times to the subject after administration of the conjugate. In some of any embodiments, the immune checkpoint inhibitor is administered once, twice, three times, four times, five times, six times, seven times, eight times, nine times, 10 times, or more than 10 times after the administration of the conjugate to the subject.

In some of any embodiments, the anti-PD-1 antibody is administered to the subject concurrently with the conjugate. In some of any embodiments, the anti-PD-1 antibody is administered to the subject within 24 to 48 hours of administering the conjugate. In some of any embodiments, the anti-PD-1 antibody is administered to the subject within 24 hours±4 hours of administering the conjugate.

In some of any embodiments, a first dose of the anti-PD-1 antibody is administered prior to the administration of the conjugate. In some of any embodiments, the first dose of the anti-PD-1 antibody is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

In some of any embodiments, the anti-PD-1 antibody is administered once, twice, three times, four times, five times, six times, seven times, eight times, nine times, 10 times, or more than 10 times to the subject. In some of any embodiments, anti-PD-1 antibody is administered once, twice, three times, four times, five times, six times, seven times, eight times, nine times, or 10 times before the administration of the conjugate, and subsequently followed by the administration of the anti-PD-1 antibody zero times, once, twice, three times, four times, five times, six times, seven times, eight times, nine times, 10 times, or more than 10 times to the subject after administration of the conjugate. In some of any embodiments, the anti-PD-1 antibody is administered concurrently with the administration of the conjugate, and subsequently followed by the administration of the anti-PD-1 antibody zero times, once, twice, twice, three times, four times, five times, six times, seven times, eight times, nine times, 10 times, or more than 10 times to the subject after administration of the conjugate. In some of any embodiments, the anti-PD-1 antibody is administered once, twice, three times, four times, five times, six times, seven times, eight times, nine times, 10 times, or more than 10 times after the administration of the conjugate to the subject.

In some of any embodiments, the secondary population is comprised in a solid tumor.

In some of any embodiments, the subject exhibits a complete response.

In some of any embodiments, the anti-CD25 antibody comprises a functional Fc region. In some of any embodiments, the anti-CD25 antibody is basiliximab or daclizumab, or biosimilar, interchangeable, biobetter, copy biological or biogeneric thereof. In some of any embodiments, the anti-CD25 antibody is basiliximab.

In some of any embodiments, the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

In some of any embodiments, the phthalocyanine dye is a Si-phthalocyanine dye. In some of any embodiments, the Si-phthalocyanine dye is IR700.

In some of any embodiments, the illumination is carried out between 30 minutes and 96 hours after administering the conjugate. In some of any embodiments, the illumination is carried out 24 hours±4 hours after administering the conjugate. In some of any embodiments, the first tumor is illuminated at a wavelength of 690±40 nm. In some of any embodiments, the first tumor is illuminated at a dose of or about of 50 J/cm² or 100 J/cm of fiber length.

In some of any embodiments, the methods and uses also involve (d) administering an additional therapeutic agent or anti-cancer treatment.

In some of any embodiments, one or more of steps (a), (b), (c), or (d) are repeated.

In some of any embodiments, the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some of any embodiments, the second population is located in one, two, three, or more than three tissues or organs that are different from the tissue or organ where the first tumor is located.

In some embodiments, the first tumor and/or the secondary population is inhibited to a greater degree as compared with administration of only the conjugate followed by illumination, and as compared with administration of only the immune checkpoint inhibitor. In some embodiments, the first tumor and/or the secondary population is inhibited to a greater degree as compared with administration of only the conjugate followed by illumination, and as compared with administration of only the anti-PD-1 antibody.

Provided herein are methods and uses for generating an enhanced response in a subject having a cancer. In some of any embodiments, the methods and uses involve: administering an immune checkpoint inhibitor to the subject having a cancer comprising a tumor; administering a conjugate comprising a phthalocyanine dye linked to an anti-CD25 antibody to the subject, wherein the immune checkpoint inhibitor is administered prior to or concurrently with the conjugate; and after administering the conjugate, illuminating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length.

In some of any embodiments: the enhanced response comprises an enhancement of systemic immunity of the subject as compared to the systemic immunity of the subject prior to the administration of the conjugate followed by illumination and the immune checkpoint inhibitor; and/or the enhanced response comprises an enhancement of inhibition of the tumor as compared with administration of only the conjugate, only the conjugate followed by illumination, or only the immune checkpoint inhibitor.

In some of any embodiments, the immune checkpoint inhibitor is selected from the group consisting of a PD-1 inhibitor, a PD-L1 inhibitor or a CTLA-4 inhibitor. In some of any embodiments, the immune checkpoint inhibitor is or comprises an anti-PD-1 antibody, an anti-PD-L1 antibody or an anti-CTLA-4 antibody, or an antigen-binding fragment thereof. In some of any embodiments, the immune checkpoint inhibitor is or comprises an anti-PD-1 antibody or an antigen-binding fragment thereof.

Provided herein are methods and uses for generating an enhanced response in a subject having a cancer. In some of any embodiments, the methods and uses involve: administering an anti-PD-1 antibody to the subject having a cancer comprising a tumor; administering a conjugate comprising a phthalocyanine dye linked to an anti-CD25 antibody to the subject, wherein the anti-PD-1 antibody is administered prior to or concurrently with the conjugate; and after administering the conjugate, illuminating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length; wherein: the enhanced response comprises an enhancement of systemic immunity of the subject as compared to the systemic immunity of the subject prior to the administration of the conjugate followed by illumination and the anti-PD-1 antibody; and/or the enhanced response comprises an enhancement of inhibition of the tumor as compared with administration of only the conjugate, only the conjugate followed by illumination, or only the anti-PD-1 antibody.

In some of any embodiments, the enhanced response is additive or synergistic.

In some of any embodiments, inhibition comprises one or more of: less than at or about 20% increase in tumor volume, tumor dimensions or tumor mass, or a reduction in tumor volume, tumor dimensions or tumor mass, or a reduction in the number of tumor cells. In some of any embodiments, the reduction in tumor volume, tumor dimensions or tumor mass, or the number of tumor cells comprises at or about a 30% reduction or more.

In some of any embodiments: the tumor comprises a first tumor and a secondary population of tumor cells, and wherein the first tumor is illuminated and the secondary population is not directly illuminated; the tumor comprises a first tumor and metastatic tumor cells, and wherein the first tumor is illuminated and the metastatic tumor cells are not directly illuminated; and/or the tumor comprises a first tumor and invasive tumor cells, and wherein the first tumor is illuminated and the invasive tumor cells are not directly illuminated.

In some of any embodiments, the enhanced response is a synergistic response, wherein the synergistic response comprises a synergistic reduction in the growth, tumor volume, tumor dimensions or tumor mass of a first tumor, a synergistic reduction in the number of cells in the secondary population in the subject, a synergistic reduction in the growth, tumor volume, tumor dimensions, tumor mass, or the number of metastatic or invasive tumor cells, or any combination thereof.

In some of any embodiments, the immune checkpoint inhibitor is administered to the subject within 24 hour to 48 hours of administering the conjugate. In some of any embodiments, the immune checkpoint inhibitor is administered to the subject within 24 hours±4 hours of administering the conjugate. In some of any embodiments, a first dose of the immune checkpoint inhibitor is administered prior to the administration of the conjugate. In some of any embodiments, the first dose of the immune checkpoint inhibitor is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

In some of any embodiments, the anti-PD-1 antibody is administered to the subject within 24 hour to 48 hours of administering the conjugate. In some of any embodiments, the anti-PD-1 antibody is administered to the subject within 24 hours±4 hours of administering the conjugate. In some of any embodiments, a first dose of the anti-PD-1 antibody is administered prior to the administration of the conjugate. In some of any embodiments, the first dose of the anti-PD-1 antibody is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

In some of any embodiments, systemic immunity is measured by one or more of a cytotoxic T lymphocyte (CTL) activity assay, an intratumoral T cell exhaustion assay, an intratumoral effector T cell expansion assay, a T cell receptor diversity assay, an activated CD8⁺ T cell assay, a circulating regulatory T cell (Treg) assay, an intratumoral Treg assay, or a CD8⁺ Tcell:Treg assay. In some of any embodiments, systemic immunity is measured by a CTL activity assay using splenocytes or peripheral blood cells or bone marrow cells or lymph node cells, optionally collected from the subject between day 4 and day 28 after illumination of the first tumor in the subject. In some of any embodiments, systemic immunity is measured by an intratumoral T cell exhaustion assay using T cells collected from the first tumor or a metastatic tumor cell mass or an invasive tumor cell mass, optionally collected from the subject between day 4 and day 28 after illumination of the first tumor in the subject. In some of any embodiments, systemic immunity is measured by an intratumoral effector T cell expansion assay using T cells collected from the first tumor or a metastatic tumor cell mass or an invasive tumor cell mass, optionally collected from the subject between day 4 and day 28 after illumination of the first tumor in the subject. In some of any embodiments, systemic immunity is measured by a T cell receptor diversity assay using T cells collected from the first tumor or a metastatic tumor cell mass or an invasive tumor cell mass or the peripheral circulation, optionally collected from the subject between day 4 and day 28 after illumination of the first tumor in the subject.

In some of any embodiments, systemic immunity is measured by determining the presence, number or frequency of regulatory T cells (Tregs) in the tumor and/or the ratio of intratumoral Treg cells to intratumoral CD8+ T cells or intratumoral CD4+ T cells from the first tumor or a metastatic tumor cell mass or an invasive tumor cell mass, optionally collected from the subject between day 4 and day 28 after illumination of the first tumor in the subject.

In some of any embodiments, the anti-CD25 antibody comprises a functional Fc region. In some of any embodiments, the anti-CD25 antibody is basiliximab or daclizumab, or biosimilar, interchangeable, biobetter, copy biological or biogeneric thereof. In some of any embodiments, the anti-CD25 antibody is basiliximab.

In some of any embodiments, the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

In some of any embodiments, the immune checkpoint inhibitor is administered once, twice, three times, four times, five times, six times, seven times, eight times, nine times, 10 times, or more than 10 times to the subject. In some of any embodiments, the anti-PD-1 antibody is administered once, twice, three times, four times, five times, six times, seven times, eight times, nine times, 10 times, or more than 10 times to the subject.

In some of any embodiments, the phthalocyanine dye is a Si-phthalocyanine dye. In some of any embodiments, the Si-phthalocyanine dye is IR700.

In some of any embodiments, the illumination is carried out between 30 minutes and 96 hours after administering the conjugate. In some of any embodiments, the illumination is carried out 24 hours±4 hours after administering the conjugate. In some of any embodiments, the tumor is illuminated at a wavelength of 690±40 nm. In some of any embodiments, the tumor is illuminated at a dose of or about of 50 J/cm² or 100 J/cm of fiber length.

In some of any embodiments, the methods and uses also involve (d) administering an additional therapeutic agent or anti-cancer treatment.

In some of any embodiments, one or more of steps (a), (b), (c), or (d) are repeated.

In some of any embodiments, the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some of any embodiments, the enhanced response comprises an additive response or a synergistic response as compared with administration of only the conjugate followed by illumination, and as compared with administration of only the immune checkpoint inhibitor. In some of any embodiments, the enhanced response comprises an additive response or a synergistic response as compared with administration of only the conjugate followed by illumination, and as compared with administration of only the anti-PD-1 antibody.

Provided in some embodiments are methods for treating cancer in a subject using photoimmunotherapy, e.g., with a photosensitive phthalocyanine dye-targeting molecule conjugate. In some embodiments, the method includes administering to a subject having cancer, such as cancers that include a first tumor or primary tumor and invasive tumor cells and/or metastatic tumor cells, a conjugate containing a phthalocyanine dye linked to a targeting molecule, such as an antibody or an antigen-binding fragment thereof, that binds to CD25 protein on the surface of a cell present in tumor microenvironment. In some embodiments, the conjugate is administered alone or in combination with an immune checkpoint inhibitor (ICI). In some embodiments, after administering the conjugate, the first tumor or primary tumor is illuminated at a wavelength of 500 to 900 nm at a dose of at least 1 J/cm² or 1 J/cm of fiber length thereby treating the tumor in the subject. In some embodiments, the wavelength for illumination is 600 nm to 850 nm, such as 660 nm to 740 nm.

In some embodiments, the cancer includes first tumor or primary tumor or multiple first tumor or primary tumors, invasive tumor cells, and/or metastatic tumor cells.

Provided are methods and uses for treating a cancer. In some of any embodiments, the methods and uses involve: (a) administering to a subject having a cancer comprising a first tumor or primary tumor and metastatic tumor cells, a conjugate comprising a phthalocyanine dye linked to a targeting molecule, wherein the targeting molecule binds to CD25, (b) administering an immune checkpoint inhibitor to the subject, and (c) after administering the conjugate, illuminating the first tumor or primary tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the growth and/or increase in volume of one or both of the first tumor or primary tumor and metastatic tumor cells in the subject is inhibited. In some of any embodiments, the metastatic tumor cells are not directly illuminated.

In some of any embodiments, the metastatic tumor cells are comprised in a solid tumor. In some of any embodiments, the growth inhibition of the first tumor or primary tumor, the metastatic tumor cells or both is synergistic as compared with administration of only one of the conjugate and the immune checkpoint inhibitor.

In some of any embodiments, the subject exhibits a complete response.

In some of any embodiments, the targeting molecule is an antibody or antigen-binding fragment thereof or includes an antigen-binding fragment. In some of any embodiments, the antibody is an anti-CD25 antibody. In some of any embodiments, the anti-CD25 antibody includes a functional Fc region. In some of any embodiments, the anti-CD25 antibody is basiliximab or daclizumab, or biosimilar, interchangeable, biobetter, copy biological or biogeneric thereof. In some of any embodiments, the anti-CD25 antibody is basiliximab.

In some of any embodiments, the immune checkpoint inhibitor is selected from the group consisting of a PD-1 inhibitor, a PD-L1 inhibitor or a CTLA-4 inhibitor. In some of any embodiments, the immune checkpoint inhibitor is or includes an anti-PD-1 antibody, an anti-PD-L1 antibody or an anti-CTLA-4 antibody, or an antigen-binding fragment thereof.

In some of any embodiments, the immune checkpoint inhibitor is or includes an anti-PD-1 antibody or an antigen-binding fragment thereof. In some of any embodiments, the anti-PD-1 antibody is selected from the group consisting of pembrolizumab (MK-3475, KEYTRUDA), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab.

In some of any embodiments, the anti-PD-L1 antibody is selected from the group consisting of atezolizumab (MPDL3280A, Tecentriq), avelumab (BAVENCIO), durvalumab (MEDI4736, IMFINZI), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

In some of any embodiments, the anti-CTLA-4 antibody is selected from the group consisting of ipilimumab (YERVOY), tremelimumab, AGEN1181, AGEN1884, ADU-1064, BCD-145, and BCD-217.

In some of any embodiments, the conjugate is administered to the subject before, concurrently, or after the administration of the immune checkpoint inhibitor.

In some of any embodiments, the immune checkpoint inhibitor is administered once, twice, three times, four times, five times, six times, seven times, eight times, nine times, 10 times, or more than 10 times to the subject. In some of any embodiments, the immune checkpoint inhibitor is administered once, twice, three times, four times, five times, six times, seven times, eight times, nine times, or 10 times before the administration of the conjugate, and subsequently followed by the administration of the immune checkpoint inhibitor zero time, once, twice, three times, four times, five times, six times, seven times, eight times, nine times, 10 times, or more than 10 times to the subject after administration of the conjugate. In some of any embodiments, the immune checkpoint inhibitor is administered concurrently with the administration of the conjugate, and subsequently followed by the administration of the immune checkpoint inhibitor zero time, once, twice, twice, three times, four times, five times, six times, seven times, eight times, nine times, 10 times, or more than 10 times to the subject after administration of the conjugate. In some of any embodiments, the immune checkpoint inhibitor is administered once, twice, three times, four times, five times, six times, seven times, eight times, nine times, 10 times, or more than 10 times after the administration of the conjugate to the subject.

In some of any embodiments, the phthalocyanine dye is a Si-phthalocyanine dye. In some of any embodiments, the Si-phthalocyanine dye is IR700.

In some of any embodiments, the illumination is carried out between 30 minutes and 96 hours after administering the conjugate. In some of any embodiments, the illumination is carried out 24 hours±4 hours after administering the conjugate. In some of any embodiments, the first tumor or primary tumor is illuminated at a wavelength of 690±40 nm. In some of any embodiments, the first tumor or primary tumor is illuminated at a dose of or about of 50 J/cm² or 100 J/cm of fiber length.

In some of any embodiments, one or more of steps (a), (b) or (c) are repeated.

In some of any embodiments, the provided methods or uses further involve (d) administering an additional therapeutic agent or anti-cancer treatment.

In some of any embodiments, the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma. In some of any embodiments, the metastatic tumor cells are located in one, two, three, or more than three tissues or organs that are different from the tissue or organ where the first tumor or primary tumor is located.

Provided are methods and uses for treating a cancer that involve: (1) administering to a subject having a cancer that includes a first tumor or primary tumor and invasive tumor cells, a conjugate comprising a phthalocyanine dye linked to a targeting molecule, wherein the targeting molecule binds to CD25, (2) administering an immune checkpoint inhibitor to the subject, and (3) after administering the conjugate, illuminating the first tumor or primary tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the growth and/or increase in volume of one or both of the first tumor or primary tumor and invasive tumor cells in the subject is inhibited. In some of any embodiments, the invasive tumor cells are not directly illuminated.

Provided are methods and uses for treating a cancer. In some of any embodiments, the methods and uses involve: the invasive tumor cells are comprised in a solid tumor.

In some of any embodiments, the growth inhibition of the first tumor or primary tumor, the invasive tumor cells or both is synergistic as compared with administration of only one of the conjugate and the immune checkpoint inhibitor.

In some of any embodiments, the subject exhibits a complete response.

In some of any embodiments, the targeting molecule is an antibody or antigen-binding fragment thereof or includes an antigen-binding fragment. In some of any embodiments, the antibody is an anti-CD25 antibody. In some of any embodiments, the anti-CD25 antibody includes a functional Fc region. In some of any embodiments, the anti-CD25 antibody is basiliximab or daclizumab, or biosimilar, interchangeable, biobetter, copy biological or biogeneric thereof. In some of any embodiments, the anti-CD25 antibody is basiliximab.

In some of any embodiments, the immune checkpoint inhibitor is selected from the group consisting of a PD-1 inhibitor, a PD-L1 inhibitor or a CTLA-4 inhibitor. In some of any embodiments, the immune checkpoint inhibitor is or includes an anti-PD-1 antibody, an anti-PD-L1 antibody or an anti-CTLA-4 antibody, or an antigen-binding fragment thereof.

In some of any embodiments, the immune checkpoint inhibitor is or includes an anti-PD-1 antibody or an antigen-binding fragment thereof. Provided are methods and uses for treating a cancer. In some of any embodiments, the methods and uses involve: the anti-PD-1 antibody is selected from the group consisting of pembrolizumab (MK-3475, KEYTRUDA), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab.

In some of any embodiments, the anti-PD-L1 antibody is selected from the group consisting of atezolizumab (MPDL3280A, Tecentriq), avelumab (BAVENCIO), durvalumab (MEDI4736, IMFINZI), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

In some of any embodiments, the anti-CTLA-4 antibody is selected from the group consisting of ipilimumab (YERVOY), tremelimumab, AGEN1181, AGEN1884, ADU-1064, BCD-145, and BCD-217.

Provided are methods and uses for treating a cancer. In some of any embodiments, the methods and uses involve: the conjugate is administered to the subject before, concurrently, or after the administration of the immune checkpoint inhibitor.

In some of any embodiments, the immune checkpoint inhibitor is administered once, twice, three times, four times, five times, six times, seven times, eight times, nine times, 10 times, or more than 10 times to the subject. In some of any embodiments, the immune checkpoint inhibitor is administered once, twice, three times, four times, five times, six times, seven times, eight times, nine times, or 10 times before administration of the conjugate, and subsequently followed by the administration of the immune checkpoint inhibitor zero time, once, twice, three times, four times, five times, six times, seven times, eight times, nine times, 10 times, or more than 10 times to the subject after administration of the conjugate. In some of any embodiments, the immune checkpoint inhibitor is administered concurrently with the administration of the conjugate, and subsequently followed by the administration of the immune checkpoint inhibitor zero time, once, twice, twice, three times, four times, five times, six times, seven times, eight times, nine times, 10 times, or more than 10 times to the subject after administration of the conjugate. In some of any embodiments, the immune checkpoint inhibitor is administered once, twice, three times, four times, five times, six times, seven times, eight times, nine times, 10 times, or more than 10 times after administration of the conjugate to the subject.

In some of any embodiments, the phthalocyanine dye is a Si-phthalocyanine dye. In some of any embodiments, the Si-phthalocyanine dye is IR700.

In some of any embodiments, the illumination is carried out between 30 minutes and 96 hours after administering the conjugate. In some of any embodiments, the illumination is carried out 24 hours±4 hours after administering the conjugate.

In some of any embodiments, the first tumor or primary tumor is illuminated at a wavelength of 690±40 nm. In some of any embodiments, the first tumor or primary tumor is illuminated at a dose of or about of 50 J/cm² or 100 J/cm of fiber length.

In some of any embodiments, one or more of steps (1), (2) or (3) are repeated.

In some of any embodiments, the provided methods and uses also involve (4) administering an additional therapeutic agent or anti-cancer treatment.

In some of any embodiments, the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

Also provided are methods and uses for enhancing systemic immunity in a subject having a tumor. In some of any embodiments, the methods and uses involve: (a) administering to a subject having a tumor a conjugate comprising a phthalocyanine dye linked to an anti-CD25 antibody, (b) administering an immune checkpoint inhibitor to the subject, and (c) after administering the conjugate, illuminating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein systemic immunity of the subject is enhanced as compared to the systemic immunity of the subject prior to the administration of the conjugate and the immune checkpoint inhibitor.

In some of any embodiments, systemic immunity is measured by one or more of a cytotoxic T lymphocyte (CTL) activity assay, an intratumoral T cell exhaustion assay, an intratumoral effector T cell expansion assay, or a T cell receptor diversity assay.

In some of any embodiments, the tumor includes a first tumor or primary tumor and metastatic tumor cells, and wherein the first tumor or primary tumor is illuminated and the metastatic tumor cells are not directly illuminated; or the tumor includes a first tumor or primary tumor and invasive tumor cells, and wherein the first tumor or primary tumor is illuminated and the invasive tumor cells are not directly illuminated.

In some of any embodiments, systemic immunity is measured by a CTL activity assay using splenocytes or peripheral blood cells or bone marrow cells or lymph node cells, optionally collected from the subject between day 4 and day 28 after illumination of the first tumor or primary tumor in the subject.

In some of any embodiments, systemic immunity is measured by an intratumoral T cell exhaustion assay using T cells collected from the first tumor or primary tumor or a metastatic tumor cell mass or an invasive tumor cell mass, optionally collected from the subject between day 4 and day 28 after illumination of the first tumor or primary tumor in the subject.

In some of any embodiments, systemic immunity is measured by an intratumoral effector T cell expansion assay using T cells collected from the first tumor or primary tumor or a metastatic tumor cell mass or an invasive tumor cell mass, optionally collected from the subject between day 4 and day 28 after illumination of the first tumor or primary tumor in the subject.

In some of any embodiments, systemic immunity is measured by a T cell receptor diversity assay using T cells collected from the first tumor or primary tumor or a metastatic tumor cell mass or an invasive tumor cell mass or the peripheral circulation, optionally collected from the subject between day 4 and day 28 after illumination of the first tumor or primary tumor in the subject.

In some of any embodiments, systemic immunity is measured by determining the presence, number or frequency of regulatory T cells (Tregs) in the tumor and/or the ratio of intratumoral Treg cells to intratumoral CD8+ T cells or intratumoral CD4+ T cells from the first tumor or primary tumor or a metastatic tumor cell mass or an invasive tumor cell mass, optionally collected from the subject between day 4 and day 28 after illumination of the first tumor or primary tumor in the subject.

In some of any embodiments, the anti-CD25 antibody comprises a functional Fc region. In some of any embodiments, the anti-CD25 antibody is basiliximab or daclizumab, or biosimilar, interchangeable, biobetter, copy biological or biogeneric thereof. In some of any embodiments, the anti-CD25 antibody is basiliximab. In some of any embodiments, the immune checkpoint inhibitor is or includes an anti-PD-1 antibody, an anti-PD-L1 antibody or an anti-CTLA-4 antibody, or an antigen-binding fragment thereof.

In some of any embodiments, the immune checkpoint inhibitor is or includes an anti-PD-1 antibody or an antigen-binding fragment thereof. In some of any embodiments, the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

In some of any embodiments, the anti-PD-L1 antibody is selected from the group consisting of atezolizumab (MPDL3280A, Tecentriq), avelumab (BAVENCIO), durvalumab (MEDI4736, IMFINZI), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

In some of any embodiments, the anti-CTLA-4 antibody is selected from the group consisting of ipilimumab (YERVOY), tremelimumab, AGEN1181, AGEN1884, ADU-1064, BCD-145, and BCD-217.

In some of any embodiments, the conjugate is administered to the subject before, concurrently with, or after the administration of the immune checkpoint inhibitor. In some of any embodiments, the immune checkpoint inhibitor is administered once, twice, three times, four times, five times, six times, seven times, eight times, nine times, 10 times, or more than 10 times to the subject.

In some of any embodiments, the phthalocyanine dye is a Si-phthalocyanine dye. In some of any embodiments, the Si-phthalocyanine dye is IR700.

In some of any embodiments, the illumination is carried out between 30 minutes and 96 hours after administering the conjugate and immune checkpoint inhibitor. In some of any embodiments, the illumination is carried out 24 hours±4 hours after administering the conjugate and immune checkpoint inhibitor.

In some of any embodiments, the tumor is illuminated at a wavelength of 690±40 nm. In some of any embodiments, the tumor is illuminated at a dose of or about of 50 J/cm2 or 100 J/cm of fiber length.

In some of any embodiments, one or more of steps (a), (b), (c) or (d) are repeated.

In some of any embodiments, the tumor is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

Also provided are methods and uses for generating a synergistic response in a subject. In some embodiments, the methods and uses involve: (1) administering to a subject having cancer that includes a first tumor or primary tumor a conjugate comprising a phthalocyanine dye linked to a targeting molecule, wherein the targeting molecule binds to CD25, (2) administering an immune checkpoint inhibitor to the subject, and (3) after administering the conjugate, illuminating the first tumor or primary tumor at a wavelength of at or about 600 nm to at or about 850 nm and at a dose of from at or about 25 J/cm2 to at or about 400 J/cm2 or from at or about 2 J/cm fiber length to at or about 500 J/cm fiber length, wherein the growth and/or increase in volume of the first tumor or primary tumor is synergistically reduced as compared to administration of only the conjugate or only the immune checkpoint inhibitor.

In some of any embodiments, the cancer further includes metastatic tumor cells, and wherein the growth and/or increase in volume of the metastatic tumor cells is reduced as compared to administration of only the conjugate or only the immune checkpoint inhibitor. In some of any embodiments, the reduction of growth or the increase in volume of the metastatic tumor cells is synergistic.

In some of any embodiments, the cancer further includes invasive tumor cells, and wherein the growth and/or increase in volume of the invasive tumor cells is reduced as compared to administration of only the conjugate or only the immune checkpoint inhibitor. In some of any embodiments, the reduction of growth or the increase in volume of the invasive tumor cells is synergistic.

In some of any embodiments, the targeting molecule is an antibody or antigen-binding fragment thereof or includes an antigen-binding fragment. In some of any embodiments, the antibody is an anti-CD25 antibody. In some of any embodiments, the anti-CD25 antibody includes a functional Fc region. In some embodiments, the anti-CD25 antibody is an antibody fragment. In some of any of the embodiments, the anti-CD25 antibody is basiliximab or daclizumab, or biosimilar, interchangeable, biobetter, copy biological or biogeneric thereof. In some of any embodiments, the anti-CD25 antibody is basiliximab. In some of any embodiments, the immune checkpoint inhibitor is selected from the group consisting of a PD-1 inhibitor, a PD-L1 inhibitor or a CTLA-4 inhibitor. In some of any embodiments, the immune checkpoint inhibitor is or includes an anti-PD-1 antibody, an anti-PD-L1 antibody or an anti-CTLA-4 antibody, or an antigen-binding fragment thereof.

In some of any embodiments, the immune checkpoint inhibitor is or includes an anti-PD-1 antibody or an antigen-binding fragment thereof. In some of any embodiments, the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

In some of any embodiments, the anti-PD-L1 antibody is selected from the group consisting of atezolizumab (MPDL3280A, Tecentriq), avelumab (BAVENCIO), durvalumab (MEDI4736, IMFINZI), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

In some of any embodiments, the anti-CTLA-4 antibody is selected from the group consisting of ipilimumab (YERVOY), tremelimumab, AGEN1181, AGEN1884, ADU-1064, BCD-145, and BCD-217.

In some of any embodiments, the conjugate is administered to the subject before, concurrently with, or after the administration of the immune checkpoint inhibitor. In some of any embodiments, the immune checkpoint inhibitor is administered once, twice, three times, four times, five times, six times, seven times, eight times, nine times, 10 times, or more than 10 times to the subject. In some of any embodiments, the immune checkpoint inhibitor is administered once, twice, three times, four times, five times, six times, seven times, eight times, nine times, or 10 times before the administration of the conjugate, and subsequently followed by the administration of the immune checkpoint inhibitor zero time, once, twice, three times, four times, five times, six times, seven times, eight times, nine times, 10 times, or more than 10 times to the subject after administration of the conjugate. In some of any embodiments, the immune checkpoint inhibitor is administered concurrently with the administration of the conjugate, and subsequently followed by the administration of the immune checkpoint inhibitor zero time, once, twice, twice, three times, four times, five times, six times, seven times, eight times, nine times, 10 times, or more than 10 times to the subject after administration of the conjugate. In some of any embodiments, the immune checkpoint inhibitor is administered once, twice, three times, four times, five times, six times, seven times, eight times, nine times, 10 times, or more than 10 times after the administration of the conjugate to the subject.

In some of any embodiments, the phthalocyanine dye is a Si-phthalocyanine dye. 1 In some of any embodiments, the Si-phthalocyanine dye is IR700.

In some of any embodiments, the illumination is carried out between 30 minutes and 96 hours after administering the conjugate. In some of any embodiments, the illumination is carried out 24 hours±4 hours after administering the conjugate.

In some of any embodiments, the first tumor or primary tumor is illuminated at a wavelength of 690±40 nm. In some of any embodiments, the first tumor or primary tumor is illuminated at a dose of or about of 50 J/cm2 or 100 J/cm of fiber length.

In some of any embodiments, one or more of steps (1), (2), and (3) are repeated.

In some of any embodiments, the methods and uses also involve (4) administering an additional therapeutic agent or anti-cancer treatment.

In some of any embodiments, the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some of any embodiments, the metastatic tumor cells are located in one, two, three, or more than three tissues or organs that are different from the tissue or organ where the first tumor or primary tumor is located.

### BRIEF DESCRIPTION OF THE DRAWINGS

FIGS. 1A-1B show the in vivo change in tumor volume (FIG. 1A) and a survival curve (FIG. 1B) in a mouse CT26-EphA2 clone c4D10 allograft model after treatment with an anti-PD-1 antibody (RMP1-14), PC61-IR700 (anti-CD25 antibody-IR700 conjugate, no illumination), PC61-IR700 PIT (anti-CD25 antibody-IR700 conjugate and illumination), the combination of PC61-IR700 PIT and anti-PD-1 antibody, and saline control.

FIGS. 2A-2B show the in vivo change in tumor volume of illuminated primary tumors (FIG. 2A) and unilluminated, distal tumors (FIG. 2B) after treatment with anti-PD-1 antibody (RMP1-14), PC61-IR700, PC61-IR700 PIT, and the combination of PC61-IR700 PIT and anti-PD-1 antibody.

FIGS. 3A-3B show the effects of an exemplary anti-CD25 antibody with a wild-type rat Fc region (PC61 rat WT), a wild-type mouse Fc region (PC61 mouse WT) or an N297Q mutant mouse Fc region (PC61 mouse N297Q), on circulating regulatory T cells (Tregs) in vivo at 1 day (FIG. 3A) and 8 days (FIG. 3B) post-antibody administration.

FIGS. 4A-4B show the synergistic effect of anti-PD-1 and anti-CD25 (wild-type Fc; mWT PC61)-IR700 PIT (FIG. 4A) and anti-PD-1 and N297Q Fc mutant anti-CD25 (N297Q PC61)-IR700 PIT (FIG. 4B) on non-illuminated, distal tumors.

FIG. 5 shows the percentage of intratumoral CD3⁺ CD4⁺ FoxP3⁺ regulatory T (T_{reg}) cells among CD45⁺ cells, from mice with a tumor that have been treated with an anti-CD25-IR700 with illumination (PIT), anti-CD25-IR700 conjugate without illumination (conjugate) or saline control.

FIG. 6A shows a comparison of intratumoral CD8⁺ T cells in mice treated with anti-CD25-IR700 PIT and saline and conjugate (conj.) only (no illumination) controls. FIG. 6B shows a comparison of intratumoral exhausted CD8⁺ T cells (in mice treated with anti-CD25-IR700 PIT as compared to saline and conjugate (conj.) only (no illumination) controls. FIG. 6C shows a comparison of intratumoral effector CD8⁺ T cells in mice treated with anti-CD25-IR700 PIT and saline and conjugate (conj.) only (no illumination) controls.

FIG. 7 shows the effect of anti-CD25 PIT on intratumoral CD8⁺ T cell activation in vivo, from mice with a tumor that have been treated with an anti-CD25-IR700 with illumination (PIT), anti-CD25-IR700 conjugate without illumination (conjugate) or saline control.

FIGS. 8A-8B show the ratio of intratumoral total CD8⁺ T cells (CD3⁺ CD8⁺) to CD3⁺CD4⁺FoxP3⁺ Tregs from tumor-bearing mice that have been treated with anti-CD25-IR700 with illumination (PIT), anti-CD25-IR700 conjugate without illumination (conj.), or saline control, at 2 hours (FIG. 8A) or 8 days after illumination (FIG. 8B) after illumination.

FIG. 9 shows the results for a contralateral site tumor re-challenge in animals previously treated with an anti-CD25 antibody-IR700 and illumination (PC61-IR700 PIT) or a combination of PC61-IR700 PIT and an anti-PD-1 antibody (RMP1-14) (both of which resulted in complete response after the initial treatment), and the changes in tumor volume in naïve (control) animals.

FIG. 10 shows the results for a tumor re-challenge with a different tumor type (4T1), in mice that have been previously treated with a combination of an anti-CD25 antibody-IR700 and illumination (PC61-IR700 PIT) and an anti-PD-1 antibody (RMP1-14), which exhibited a complete response after a re-challenge with the same tumor type as the initial tumor, and the changes in tumor volume in naïve (control) animals.

FIG. 11 shows the cytotoxicity against CT26 tumor cells or unrelated tumor cells after incubation with splenocytes obtained from complete response (CR) mice that were treated with an anti-CD25 antibody-IR700 and illumination (PC61-IR700 PIT) or a combination of PC61-IR700 PIT and anti-PD-1 antibody (RMP1-14) at effector: target ratios of 30:1, 15:1, and 7:1 (or 250:1 for unrelated tumor cells), after being primed with tumor specific antigen

FIGS. 12A-12B show the in vivo change in tumor volume of illuminated primary tumors (FIG. 12A) and unilluminated distal tumors (FIG. 12B) after treatment with an anti-PD-1 antibody (RMP1-14), an anti-CD25 antibody-IR700 and illumination (PC61-IR700 PIT), the combination of PC61-IR700 PIT and an anti-PD-1 antibody, and the combination of PC61-IR700 PIT and an anti-PD-1 antibody under a condition of CD8⁺ T cell depletion.

FIG. 13A shows the percentage of total FoxP3⁺ Tregs among intratumoral CD3+CD4⁺ T cells from mice with a tumor that have been treated with anti-CD25-IR700 conjugate PIT (CD25 PIT), anti-CD25-IR700 conjugate without illumination (CD25 Conj.), anti-CD25-IR700 conjugate PIT with an anti-PD-1 antibody (CD25 PIT+PD1), anti-CD25-IR700 conjugate without illumination with an anti-PD-1 antibody (CD25 Conj.+PD1) and saline control. FIG. 13B shows the ratio of intratumoral CD4⁺FoxP3⁻ helper T cell to CD4⁺FoxP3⁺ Treg cells from mice with a tumor that have been treated with anti-CD25-IR700 conjugate PIT (CD25 PIT), anti-CD25-IR700 conjugate without illumination (CD25 Conj.), anti-CD25-IR700 conjugate PIT with an anti-PD-1 antibody (CD25 PIT+PD1), anti-CD25-IR700 conjugate without illumination with an anti-PD-1 antibody (CD25 Conj.+PD1) and saline control. FIG. 13C shows the ratio of intratumoral CD8⁺ T cell to FoxP3⁺ Treg cells from mice with a tumor that have been treated with anti-CD25-IR700 conjugate PIT (anti-CD25-IR700 PIT), anti-CD25-IR700 conjugate without illumination (anti-CD25-IR700), anti-CD25-IR700 conjugate PIT with an anti-PD-1 antibody (anti-CD25-IR700 PIT+anti-PD1), anti-CD25-IR700 conjugate without illumination with an anti-PD-1 antibody (anti-CD25-IR700+anti-PD1) and saline control.

FIG. 14 shows a schematic of a proposed mechanism of action for depletion of intratumoral Treg cells by anti-CD25-IR700 PIT.

### DETAILED DESCRIPTION

Provided herein are compositions, combinations and methods for treating a cancer, such as cancers that include a first tumor or a primary tumor or multiple primary tumors as well as metastatic tumor cells, for example metastatic cancers; and/or cancers that include a primary tumor or multiple primary tumors as well as invasive or infiltrating tumor cells, for example, invasive cancers or infiltrating cancers. Also provided are compositions, combinations and methods for enhancing systemic immunity in a subject, such as a subject having a cancer, such as an invasive cancer, an infiltrating cancer, or a metastatic cancer. Also provided are compositions, combinations and methods for generating an enhanced response, for example, an enhanced response to a treatment or a therapy in a subject, e.g., a subject having a cancer or a tumor, such as an invasive cancer, an infiltrating cancer, or a metastatic cancer.

The provided compositions, combinations, methods and uses can be used to treat cancers that include a first tumor or a primary tumor, metastatic tumor cells and/or invasive tumor cells. In some embodiments, a phthalocyanine dye conjugated with a targeting molecule that binds CD25 is used alone or in combination with an immune checkpoint inhibitor. The methods and uses described herein provide various advantages in treating cancers, e.g., metastatic cancers and/or invasive cancers, including without the need to locate and/or directly illuminate the metastatic tumor cells and/or invasive tumor cells. The disclosure also provides unexpected features in enhancing the systemic immunity in a subject, for example, against cancer recurrence.

The provided embodiments, in some contexts, are based on the observation that treatment of a cancer with a phthalocyanine dye-targeting molecule conjugate, such as an anti-CD25 antibody-IR700 conjugate, followed by illumination of a first tumor or a primary tumor, results in not only treatment of the illuminated tumor, such as the illuminated first tumor or the illuminated primary tumor, but also results in effective treatment of a tumor that is distal to the illumination site (e.g., metastasized tumor), and effective treatment of a tumor that is introduced after the subject has a complete response following the treatment of the initial tumor, indicating a tumor-specific immune memory response. The provided embodiments are based on a further observation that a combination treatment with an anti-CD25 antibody-IR700 conjugate and an immune checkpoint inhibitor, such as an anti-PD-1 antibody, results in striking synergistic effects in the treatment of both the illuminated first tumor or primary tumor and a distal tumor or a later-introduced tumor, such as a tumor comprising a secondary population of tumor cells, a metastatic tumor and/or an invasive tumor. Accordingly, the provided compositions, combinations, methods and uses are demonstrated to provide a substantially improved and effective treatment of a cancer, including cancers that include a first tumor or a primary tumor or multiple primary tumors as well as metastatic tumor cells, for example metastatic cancers; and/or cancers that include a first tumor or a primary tumor or multiple primary tumors as well as invasive tumor cells, for example, invasive cancers. The provided compositions, combinations, methods and uses can result in enhancement or improvement of the subject's immune response, e.g. systemic immune response against a cancer including immune memory response, that can be effective against tumors that may develop after the treatment.

The methods and uses provided herein include treating a subject that has one or more first tumors, e.g., primary tumors, and optionally, secondary population of cells, such as metastatic tumor cells and/or invasive tumor cells with a conjugate comprising a phthalocyanine dye linked to a targeting molecule, such as a targeting molecule that binds to CD25, and after administration of the conjugate, illuminating the one or more first tumors or primary tumors with a light wavelength suitable for use with the phthalocyanine dye. Some embodiments of the method include administering an immune checkpoint inhibitor prior to, concurrent with or subsequent to administration of the conjugate.

### I. Methods and Uses for Treating a Cancer, e.g., Invasive Cancer or Metastatic Cancer

In some embodiments, provided are methods for using and uses of the compositions containing a phthalocyanine dye-targeting molecule conjugate, in which the targeting molecule binds CD25 (e.g., an anti-CD25 antibody-IR700 conjugate), for therapy or treatment of a cancer, such as a cancer that includes a first tumor or multiple tumors (e.g., one or more primary tumors) as well as a secondary population of cancer cells, such as metastatic tumor cells (e.g., metastatic cancers), invasive tumor cells, (e.g., invasive cancers), or infiltrating tumor cells (e.g., infiltrating cancers). In some embodiments, the secondary population of cancer cells is related, such as directly or indirectly, to the first tumor. In some embodiments, the secondary population of cells is not directly derived from the first tumor. The provided methods and uses include therapeutic methods and uses, for example, involving administration of the conjugates to a subject having a cancer followed by illumination (or irradiation) of a tumor associated with the cancer (such as a first tumor) or the microenvironment of a tumor, using a particular light wavelength and dose. In some aspects, the illumination (or irradiation) results in illumination-dependent lysis and death of cells expressing the target molecule (e.g., CD25), resulting in a therapeutic effect or treatment of the cancer (in some cases called photoimmunotherapy (PIT)). In some aspects, the methods also involve administration of an immune modulating agent, such as an immune checkpoint inhibitor (e.g., anti-PD-1 antibody), in combination with the phthalocyanine dye-targeting molecule conjugate. In some aspects, a combination of a phthalocyanine dye-targeting molecule conjugate and the immune checkpoint inhibitor (e.g., anti-PD-1 antibody) are employed in the provided methods and uses, such as in the provided methods and uses for treatment of a cancer.

Uses include uses of the compositions and combinations in such methods and treatments and uses of such compositions and combinations in the preparation of a medicament in order to carry out such therapeutic methods. In some embodiments, the methods and uses thereby treat the cancer, such as cancers that include tumors and cancers that include a first tumor, that is primary or non-primary tumor, and one or more secondary population(s) of tumor cells (e.g., metastatic tumor cells and/or invasive tumor cells), such as metastatic and/or invasive cancers, in a subject. In some embodiments the secondary tumor cells are related to the first tumor. In some embodiments of the methods and uses more than one tumor is treated. In some aspects, also provided are methods and uses of such compositions and combinations in enhancing, boosting, augmenting, strengthening, increasing, boosting or supporting the immune function, such as systemic immunity, in the subject.

The methods include administering to a subject having a first tumor, a conjugate comprising a phthalocyanine dye linked to targeting molecule, wherein the targeting molecule binds to CD25 and after administration of the conjugate illuminating at least a first tumor with a wavelength of light suitable for the selected phthalocyanine dye. In some embodiments, the methods include the administration of an immune checkpoint inhibitor, such as an anti-PD-1 antibody, prior to, concurrent with or subsequent to the administration of the conjugate. In some embodiments the methods further administering an additional therapeutic agent or anti-cancer treatment.

In some embodiments, the methods involve illumination of a tumor associated with a cancer or the microenvironment of a tumor (tumor microenvironment; TME), or cells that are present in the TME, with light. In some aspects, the tumor or the TME is illuminated with a wavelength of light suitable for therapy or treatment. In some embodiments, a light wavelength suitable for use with the phthalocyanine dye includes light with a wavelength that achieves activation of the dye-conjugate by illumination with absorbing light, such that the light excites the photosensitizer and results in cell killing, thereby reducing or eliminating a lesion (e.g., tumor), reducing or inhibiting tumor growth, reducing, inhibiting or eliminating secondary populations of tumor cells, such as a tumor cell metastasis, reducing, inhibiting or eliminating invasive and/or metastatic tumor cells or any combination thereof.

In some embodiments, the illumination is at a wavelength between about 500 nm and 900 nm, between about 600 nm and 850 nm, between about 650 nm and 800 nm or between about 660 nm and 740 nm. In some embodiments, illumination is at a wavelength of 690+50 nm or at a wavelength of or about 690+20 nm.

The illumination can be at a dose of at least 1 J/cm² or 1 J/cm of fiber length. In some embodiments, the lesion is illuminated at a dose of from or from about 2 J/cm² to about 400 J/cm² or from or from about 2 J/cm fiber length to about 500 J/cm fiber length. In some embodiments, the illumination is at a dose of at least or at least about 2 J/cm², 5 J/cm², 10 J/cm², 25 J/cm², 50 J/cm², 75 J/cm², 100 J/cm², 150 J/cm², 200 J/cm², 300 J/cm², 400 J/cm², or 500 J/cm²; or the lesion is illuminated at a dose of at least or at least about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the illumination is at a dose of from 25 J/cm² to 400 J/cm² or from about 2 J/cm fiber length to about 500 J/cm fiber length. In some embodiments, the illumination is at a dose from 5 J/cm² to 200 J/cm² or from 20 J/cm fiber length to 500 J/cm fiber length. In some embodiments, the illumination is at a dose of about 50 J/cm² or 100 J/cm fiber length.

In some embodiments of the methods and uses provided herein, the illumination is effected after administration of the phthalocyanine dye-targeting molecule conjugate. In some embodiments, the illumination or illumination is carried out or effected between or between about 30 minutes and 96 hours after administering the phthalocyanine dye-targeting molecule conjugate (e.g., IR700-anti-CD25 antibody conjugate), such as between 30 minutes and 48 hours, 30 minutes and 24 hours or 12 hours and 48 hours, such as generally at least 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more after administering the conjugate. In some embodiments, the illumination is performed within about 24 hours after administering the conjugate or within 24 hours±4 hours after administering the conjugate, or within about 20, 21, 22, 23, 24, 24, 26, 27, or 28 hours after administering the conjugate.

The methods described herein include illuminating a first tumor in a subject, or the tumor microenvironment (TME) of a first tumor, such as a primary tumor. In some embodiments, the methods and uses provided herein include treating a subject that has one or more tumors. The subject may have one, two, three, or more than three tumors. Such tumors can be in one or more tissues or organs, such as in one tissue or organ, in two different tissues or organs, in three different tissues or organs, or in more than three different tissues or organs.

In some aspects, primary tumor can refer to the first or original tumor in a subject and can also refer to the one or more tumors selected for illumination with the methods and uses provided herein. In some embodiments, a first tumor or additional tumors may be a solid tumor or solid tumors, may be lymphomas, or may be leukemias. The tumor can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown primary origin.

In some embodiments of the methods, the growth of the first tumor or primary tumors is inhibited, the volume of the primary tumor or primary tumors is reduced or both tumor growth and volume are reduced. In some embodiments of the methods, the growth of the first tumor or primary tumors is inhibited, the volume of the primary tumor or primary tumors is reduced or both tumor growth and volume are reduced as compared to a monotherapy such as administration of only the conjugate, only the conjugate followed by illumination, or only the anti-PD-1 antibody.

In some embodiments, the methods and uses provided herein include treating a subject that has one or more tumors and also a secondary population of tumor cells, such as invasive tumor cells. In some of such embodiments, the secondary population contains when cells originating from a tumor, such as a primary tumor, have invaded into surrounding tissues. The methods include administering to a subject having a first tumor(s) and invasive tumor cells, a conjugate comprising a phthalocyanine dye linked to targeting molecule, wherein the targeting molecule binds to CD25 and after administration of the conjugate, illuminating the first tumor with a wavelength suitable for the selected phthalocyanine dye. In some of such embodiments, the secondary population of tumor cells is not directly illuminated. In some embodiments, the methods include the administration of an immune checkpoint inhibitor prior to, concurrent with or subsequent to the administration of the conjugate. In some embodiments, the methods include the administration of an anti-PD-1 antibody prior to, concurrent with, or subsequent to administration of an anti-CD25 antibody-IR700 conjugate, whereby the conjugate administration is followed by illumination of the first tumor.

In some aspects, invasive tumor cells refer to cells originated from a primary tumor and have invaded into surrounding tissues of the same organ or neighboring organs of the primary tumor within the body of a subject having the first tumor. In some embodiments, the first tumor is a primary tumor, and the invasive tumor cells are directly or indirectly derived from the first tumor. In some embodiments, the invasive tumor cells are not directly derived from the first tumor.

The methods and uses provided herein include illumination of the first tumor and/or additional tumors, and some or all of the invasive tumor cells are not illuminated. In some embodiments, the growth of invasive tumor cells is inhibited, reduced or eliminated, the volume, dimension, or mass of one or more invasive tumors is reduced or any combination thereof. In some embodiments, the growth of the first tumor also is inhibited, reduced or eliminated, the volume, dimension, or mass of the first tumor or additional tumors also is reduced along with the effect(s) on the one or more invasive tumor cells.

In some embodiments, invasive tumor cells are contained in a solid tumor. In some embodiments, invasive tumor cells are contained in body fluids, including but not limited to peritoneal fluid, pleural fluid, and cerebrospinal fluid. In some embodiments, invasive tumor cells are contained in the effusion of a body cavity or body cavities, including but not limited to peritoneal effusion (ascites), pleural effusion, and pericardial effusion.

In some embodiments, the methods and uses provided herein include treating a subject that has a first tumor and also a secondary population of tumor cells (for example, a secondary population of related tumor cells), such as invasive and/or metastatic tumor cells. The methods include administering to a subject having a first tumor and a secondary population of tumor cells (for example, a secondary population of related tumor cells), such as invasive and/or metastatic tumor cells, a conjugate comprising a phthalocyanine dye linked to targeting molecule, wherein the targeting molecule binds to CD25 and after administration of the conjugate, illuminating the first tumor with a wavelength suitable for the selected phthalocyanine dye. In some of such embodiments, the secondary population of tumor cells is not directly illuminated. In some embodiments, the methods include the administration of an immune checkpoint inhibitor, such as an anti-PD-1 antibody, prior to, concurrent with or subsequent to the administration of the conjugate. In such methods, the growth (volume, dimension, or mass) of the first tumor and/or the secondary population of tumor cells, such as metastatic tumor cells, is inhibited, reduced or eliminated, the volume, dimension, or mass of one or more of the first tumor and/or secondary population of cells is reduced, or any combination thereof. In some embodiments, the inhibition of the first tumor and/or secondary population is effected to a greater degree than the inhibition achieved by administration of only the conjugate, only the conjugate followed by illumination, or only the anti-PD-1 antibody. In some embodiments, inhibition is achieved if the tumor exhibits less than 20% increase in tumor volume, tumor dimension(s), or tumor mass; no change in tumor volume, dimension, or mass (i.e., halted tumor growth or progression); or the tumor is reduced in volume, dimensions, or mass; or there is a reduction in the number of tumor cells. In some aspects, the reduction in tumor volume, tumor dimensions or tumor mass, or the number of tumor cells comprises at or about a 30% reduction or more.

In any of the methods and uses herein, the first tumor can be a primary tumor or a secondary tumor. In some embodiments, the first tumor and the secondary population are related. In some embodiments, the secondary population of cells is directly or indirectly derived from the first tumor. In some embodiments, the secondary population is not derived from the first tumor. In some embodiments, the first tumor is a primary tumor and the secondary population of cells is related to the primary tumor; for example, the secondary population of cells is derived directly or indirectly from the primary tumor. In some embodiments, the first tumor is a primary tumor and the secondary population of tumor cells is a second primary tumor. In some embodiments, the first tumor is a secondary tumor and the secondary population of cells is related to the secondary tumor. In some aspects, a secondary population of tumor includes cells that originated from a primary tumor and invade local or distal healthy tissue (i.e., invasive tumor cells) or spread to a distal tissue or organ, or distal tissues or organs within the body of a subject having the primary tumor (i.e., metastatic tumor cells), for example a tissue or an organ located distantly or far away from the primary tumor. In some aspects a secondary population of tumor cells is both invasive and metastatic. In some aspects a secondary population of tumor cells is infiltrating. In some aspects, a secondary population of tumor cells is metastatic and is directly or indirectly related to, such as derived from, the first tumor. In other aspects, a secondary population of tumor cells is metastatic and not directly related to the first tumor. Metastatic tumor cells can be located in one or more locations in the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, blood, bone marrow, cerebrospinal fluid, or any other tissues or organs. In some embodiments, metastatic tumor cells are contained in a solid tumor. In some embodiments, metastatic tumor cells are circulating tumor cells, are a liquid tumor, or are not associated with a tumor mass.

In some embodiments of the methods and uses provided herein, secondary tumor cells are metastatic tumor cells that are distal to the first tumor and some or all of the metastatic tumor cells are not illuminated, e.g., not directly illuminated. In some embodiments of the methods and uses, only the first tumor is illuminated after administration of the conjugate and invasive or metastatic tumor cells are not directly illuminated. In some embodiments, more than one tumor, including a first tumor, is illuminated but at least one site of tumor cells, such as a site containing metastatic tumor cells, is not illuminated.

### II. Methods for Enhancing Systemic Immunity and/or Response

Also provided herein are methods and uses of compositions and combinations in enhancing, boosting, augmenting, or supporting the immune function, such as systemic immunity, in a subject, e.g., in a subject having a cancer or a tumor. In some embodiments, the method and uses herein includes enhancing systemic immunity in a subject having a cancer, a tumor or a cancerous lesion. In some aspects, “systemic immunity” refers to the ability of a subject's immune system to respond to an immunologic challenge, including those associated with a cancer or a tumor, in a systemic manner. In some aspects, systemic immunity can include systemic response of the subject's adaptive immune system and/or innate immune system. In some aspects, systemic immunity includes an immune response across different tissues, including the blood stream, lymph node, bone marrow, spleen and/or the tumor microenvironment, and in some cases, includes a coordinated response among the tissues and organs and various cells and factors of the tissues and organs. Also provided herein are methods and uses of compositions and combinations in enhancing, boosting or augmenting the a response to a treatment or a therapy in a subject, such as in a subject having a cancer or a tumor.

In some aspects, the provide methods and uses include administering to the subject a conjugate comprising a phthalocyanine dye linked to targeting molecule, wherein the targeting molecule binds to CD25, administering an immune checkpoint inhibitor, and after administration of the conjugate, illuminating the tumor or a cancerous lesion, or the tumor microenvironment. The conditions for illumination regarding wavelength, dosage of illumination and timing of illumination are such as those described herein. The immune checkpoint inhibitor can be administered prior to, concurrent with or subsequent to the administration of the conjugate, such as described herein. In some aspects, the methods and uses provided herein result in an enhancement of the systemic immunity in the subject, which can in turn result in enhanced or synergistic response to the therapy or treatment for cancer. In some embodiments, the methods and uses provided herein results in an enhanced response, such as a synergistic response, to the treatment or therapy for the cancer or the tumor, as compared with the administration of only the conjugate, only the conjugate followed by illumination or only the anti-PD-1 antibody. In some aspects, the enhanced response comprises an enhancement of systemic immunity of the subject as compared to the systemic immunity of the subject prior to the administration of the conjugate followed by illumination and the anti-PD-1 antibody. In some aspects, the enhanced response comprises an enhanced response, such as n additional, additive, or synergistic response, and/or a more complete response, more durable response or longer lasting response, to the treatment as compared with administration of only the conjugate, only the conjugate followed by illumination, or only the anti-PD-1 antibody.

In some embodiments of the methods and uses provided herein, the systemic immunity against recurring tumors is increased or augmented. In some aspects, the level, strength or extent of systemic immunity can be measured based on the number of intratumoral CD8⁺ T lymphocytes, the ratio of CD8⁺ T lymphocytes to regulatory T cells (T_{regs}), intratumoral T lymphocyte exhaustion (e.g., the percentage of CD3⁺CD8⁺ cells that express PD-1 and/or CTLA4 markers), the number or percentage of intratumoral activated CD8⁺ T lymphocytes (e.g., Ki67⁺ or CD69⁺ CD8 cells as a percentage of CD45⁺ cells), the expansion of cytotoxic intratumoral T lymphocytes (e.g., the percentage of CD3⁺CD8⁺ cells that do not express PD-1 and/or CTLA4 markers), based on the splenocyte cytotoxicity against tumor cells, or any or all of combination thereof. In some aspects, intratumoral CD8⁺ T lymphocytes include CD3⁺CD8⁺ cells, intratumoral exhausted T lymphocytes include PD-1⁺CTLA-4⁺CD3⁺CD8⁺ cells, activated intratumoral CD8⁺ T lymphocytes include CD3⁺CD8⁺Ki67⁺ and/or CD3⁺CD8⁺CD69⁺ cells, expansion of cytotoxic T lymphocytes include PD-1⁻CTLA-4⁻CD3⁺CD8⁺ cells. In some aspects, intratumoral CD8⁺ T lymphocytes, exhausted intratumoral T lymphocytes, activated CD8⁺ T lymphocytes, or expanded cytotoxic intratumoral T lymphocytes are measured as a percentage of leukocytes (CD45⁺ cells) and/or total CD8⁺ T cells (e.g., CD3⁺CD8⁺CD45⁺ cells). Determination of such numbers or percentages can be achieved using several well-known methods, including those described herein. For example, such numbers or percentages can be determined by generating single cell suspensions, such as by mechanical dissociation of tumor and/or tissue biopsies, or collection of blood samples containing circulating immune cells, followed by staining and flow cytometric analysis or mass cytometry. Other methods can include multiplexed immunofluorescence imaging of tissue and/or tumor biopsies.

In some of such embodiments, the strength or extent of immunity is compared to the strength or extent of immunity in the same subject prior to treatment. In some of such embodiments, the strength or extent of immunity is compared to a population of subjects. In some of such embodiments, the strength or extent of immunity is compared to a threshold value. In some embodiments, the strength or extent of immunity following a combination therapy, such as anti-CD25 PIT in combination with administration of a checkpoint inhibitor (e.g., anti-PD-1 antibody), is compared to the strength or extent of immunity following treatment with a monotherapy, such as administration of a single agent, such as an immune checkpoint inhibitor (e.g., anti-PD-1 antibody) or anti-CD25 conjugate or anti-CD25 PIT alone.

In some embodiments, the strength or extent of immunity is measured by the number of intratumoral CD8⁺ T lymphocytes, such as CD3⁺CD8⁺ T lymphocytes, and the systemic immunity against recurring tumors is increased or augmented if the percentage of intratumoral CD8⁺ T lymphocytes (e.g., CD3⁺CD8⁺ T lymphocytes), among the total number of CD45⁺ cells, is increased after treatment as compared to before treatment. In some of such examples, the systemic immunity against recurring tumors is increased or augmented if the number of intratumoral CD8⁺ T lymphocytes (e.g., CD3⁺CD8⁺ T lymphocytes) is at least at or about 30% of the total number of CD45⁺ cells, such as at least at or about 30%, 35%, 36%, 37%, 38% 39%, 40%, 41%, 42% 43%, 44%, 45%, 46%, 47%, 48%, 49%, 50%, 51%, 52%, 53%, 54%, 55%, 56%, 57%, 58%, 59%, 60%, or more of the total number of CD45⁺ cells. In some embodiments, the percentage of intratumoral CD8⁺ T lymphocytes is at least 40% of the intratumoral CD45⁺ cell population. In some embodiments, the systemic immunity against recurring tumors is increased or augmented if the percentage of intratumoral CD3⁺CD8⁺ T cells among the population of intratumoral CD45⁺ cells is increased after treatment as compared to before treatment. In some of such embodiments the percentage of intratumoral CD3⁺CD8⁺ T cells among the population of intratumoral CD45⁺ cells is increased after treatment by at least at or about 5%, 6%, 7%, 8%, 9%, 10%, 11%, 12%, 13%, 14%, 15%, 16%, 17%, 18%, 19%, 20%, 25%, 30%, or more compared to before treatment. In some embodiments, the percentage of intratumoral CD3⁺CD8⁺ T cells among the population of intratumoral CD45⁺ cells is increased after treatment by at least 10% as compared to before treatment.

In some embodiments, the strength or extent of immunity is measured by the number of exhausted intratumoral CD8⁺ T lymphocytes, such as the number of PD-1⁺CTLA-4⁺CD3⁺CD8⁺ cells as a percentage of intratumoral CD8⁺ T lymphocytes (e.g., CD3⁺CD8⁺ T lymphocytes), and the systemic immunity against recurring tumors is increased or augmented if the percentage of PD-1⁺CTLA-4⁺CD3⁺CD8⁺ cells among intratumoral CD3⁺CD8⁺ T cells is decreased after treatment as comparted to before treatment. In some of such examples, the systemic immunity against recurring tumors is increased if the percentage of PD-1⁺CTLA-4⁺CD3⁺CD8⁺ cells among intratumoral CD3⁺CD8⁺ T cells after treatment is less than at or about 20%, such as less than 19%, 18%, 17%, 16%, 15%, 14%, 13%, 12%, 11%, 10%, 9%, 8%, 7%, 6%, 5%, 4%, 3%, 2%, 1%, or less. In some embodiments, the systemic immunity against recurring tumors is increased or augmented if the percentage of PD-1⁺CTLA-4⁺CD3⁺CD8⁺ cells among intratumoral CD3⁺CD8⁺ T cells is reduced after treatment compared to before treatment. In some of such embodiments, the percentage of PD-1⁺CTLA-4⁺CD3⁺CD8⁺ cells among intratumoral CD3⁺CD8⁺ T cells is decreased by at least at or about 5%, 6%, 7%, 8%, 9%, 10%, 11%, 12%, 13%, 14%, 15%, 16%, 17%, 18%, 19%, 20%, 25%, 30%, or more compared to before treatment. In some embodiments, the percentage of exhausted intratumoral CD8⁺ T cells (PD-1⁺CTLA-4⁺CD3⁺CD8⁺ cells) among the population of intratumoral CD3⁺CD8⁺ T cells is decreased after treatment by at least 10% as compared to before treatment.

In some embodiments, the strength or extent of immunity is measured by the number of activated intratumoral CD8⁺ T lymphocytes, such as CD3⁺CD8⁺Ki67⁺ and/or CD3⁺CD8⁺CD69⁺ T lymphocytes, and the systemic immunity against recurring tumors is increased or augmented if the number of activated intratumoral CD8+ T lymphocytes, such as CD3⁺CD8⁺Ki67⁺ and/or CD3⁺CD8⁺CD69⁺ T lymphocytes, as a percentage of intratumoral CD45⁺ leukocytes, is increased after treatment as compared to before treatment. In some of such embodiments, the systemic immunity against recurring tumors is increased or augmented if the number of intratumoral CD3⁺CD8⁺Ki67⁺ cells, is at least at or about 0.15% of the total number of CD45⁺ cells, such as at least at or about 0.2%, 0.25%, 0.3%, 0.35%. 0.4%, 0.45%, 0.5%, or more of the total number of intratumoral CD45⁺ cells after treatment. In other of such embodiments, the systemic immunity against recurring tumors is increased or augmented if the number of intratumoral CD3⁺CD8⁺CD69⁺ cells, is at least at or about 0.5% of the total number of CD45⁺ cells, such as at least at or about 0.6%, 0.7%, 0.8%, 0.9%. 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, or more of the total number of intratumoral CD45⁺ cells, such as at least about 1.0% of the total number of intratumoral CD45+ cells after treatment. In some embodiments, the systemic immunity against recurring tumors is increased or augmented if the percentage of intratumoral CD3⁺CD8⁺Ki67⁺ and/or CD3⁺CD8⁺CD69⁺ T lymphocytes among intratumoral CD45⁺ cells is increased after treatment compared to before treatment. In some of such embodiments, the percentage of CD3⁺CD8⁺Ki67⁺ and/or CD3⁺CD8⁺CD69⁺ T lymphocytes cells among intratumoral CD45⁺ cells is increased by at least at or about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold-18-fold, 19-fold, 20-fold, 21-fold, 22-fold, 23-fold, 24-fold, 25-fold, 30-fold, 35-fold, 40-fold, 45-fold, 50-fold, 55-fold, 60-fold, or more compared to the percentage of CD3⁺CD8⁺Ki67⁺ and/or CD3⁺CD8⁺CD69⁺ T lymphocytes cells among intratumoral CD45⁺ cells before treatment. In some embodiments, the percentage of intratumoral CD3⁺CD8⁺Ki67⁺ T lymphocytes cells among intratumoral CD45⁺ cells is increased by at least 15-fold or 20-fold compared to the percentage of CD3⁺CD8⁺Ki67⁺ T lymphocytes cells among intratumoral CD45⁺ cells before treatment. In some embodiments, the percentage of intratumoral CD3⁺CD8⁺CD69⁺ T lymphocytes cells among intratumoral CD45⁺ cells is increased by at least 5-fold compared to the percentage of CD3⁺CD8⁺CD69⁺ T lymphocytes cells among intratumoral CD45⁺ cells before treatment.

In some embodiments, the strength or extent of immunity is measured by the expansion of intratumoral cytotoxic T lymphocytes, such as PD-1⁻CTLA-4⁻CD3⁺CD8⁺ cells, and the systemic immunity against recurring tumors is increased or augmented if the percentage of intratumoral cytotoxic T lymphocytes (e.g., PD-1⁻CTLA-4⁻CD3⁺CD8⁺ cells) among CD8⁺ T cells (e.g., CD3⁺CD8⁺ T cells) is increased after treatment as compared to before treatment. In some of such examples, the systemic immunity against recurring tumors is increased or augmented if the number of intratumoral cytotoxic T lymphocytes (e.g., PD-1⁻CTLA-4⁻ CD3⁺CD8⁺ cells) is at least at or about 20% of the total number of CD3⁺CD8⁺ T cells, such as at least at or about 25%, 30%, 35%, 40%, 41%, 42% 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, or more of the total number of CD45⁺ cells. In some embodiments, the percentage of intratumoral PD-1⁻CTLA-4⁻CD3⁺CD8⁺ cells is at least at or about 40%, 45%, 50%, or 55% of the intratumoral CD3⁺CD8⁺ T cell population. In some embodiments, the systemic immunity against recurring tumors is increased or augmented if the percentage of intratumoral PD-1⁻CTLA-4⁻CD3⁺CD8⁺ cells among the population of intratumoral CD3⁺CD8⁺ T cells is increased after treatment as compared to before treatment. In some of such embodiments the percentage of intratumoral PD-1⁻CTLA-4⁻CD3⁺CD8⁺ cells among the population of intratumoral CD3⁺CD8⁺ T cells is increased after treatment by at least at or about 5%, 10%, 15%, 20%, 25%, 30%, 35%, 40%, 50%, 60%, 70%, 75%, 80%, or more compared to the percentage of intratumoral PD-1⁻CTLA-4⁻CD3⁺CD8⁺ cells among the population of intratumoral CD3⁺CD8⁺ T cells before treatment. In some embodiments, the percentage of intratumoral PD-1⁻CTLA-4⁻CD3⁺CD8⁺ cells among the population of intratumoral CD3⁺CD8⁺ T cells is increased after treatment by at least 30% as compared to the percentage of intratumoral PD-1⁻CTLA-4⁻CD3⁺CD8⁺ cells among the population of intratumoral CD3⁺CD8⁺ T cells before treatment.

In some embodiments, treatment in accordance with the methods and uses provided herein, leads to cell death of or a reduction in number of regulatory T cells (Tregs), such as intratumoral CD4+FoxP3+ Tregs. Hence, in some embodiments, the level, strength, or extent of systemic immunity can be measured based on the number or percentage of intratumoral or circulating regulatory T cells (T_{regs}). In some aspects, binding of the anti-CD25 conjugate to the surface of CD25-expressing cells, such as certain Tregs, and illumination to effect illumination-dependent lysis and death of cells expressing CD25, results in a reduction of the number of cells expressing CD25. In some aspects, such result leads to a reduction in the number of immunosuppressive cells, such as Tregs, within the tumor, and thus can alleviate or reverse immunosuppression in the tumor. In some aspects, such reduction in immunosuppressive cells can result in the activation and proliferation of intratumoral T cells, such as intratumoral CD8+ cytotoxic T cells or CD4+ helper T cells, that can eliminate tumor cells, and lead to reduction of tumor volume and/or elimination of the tumor. In some aspects, treatment in accordance with the provided embodiments can result in a reduction of intratumoral Tregs and/or an increase of intratumoral CD8+ to Treg ratio or intratumoral CD4+ to Treg ratio.

In some aspects, treatment in accordance with the methods and uses provided herein can result in a lasting or durable decrease in intratumoral Tregs. In some aspects, treatment in accordance with the methods and uses provided herein can result in a lasting or durable increase of intratumoral CD8+ to Treg ratio or intratumoral CD4+ to Treg ratio. In some embodiments, the level, strength or extent of systemic immunity can be measured by determining the intratumoral CD8⁺ to Treg ratio, and the systemic immunity against recurring tumors is increased or augmented if the intratumoral CD8⁺ to Treg ratio is increased after treatment as compared to before treatment. In some of such examples, the systemic immunity against recurring tumors is increased or augmented if the intratumoral CD8⁺ to Treg ratio is increased by at least at or about 1-fold, 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold. 4.0-fold, or more compared to the intratumoral CD8⁺ to Treg ratio before treatment. In some embodiments, the level, strength or extent of systemic immunity can be measured by determining the intratumoral CD4⁺ to Treg ratio, and the systemic immunity against recurring tumors is increased or augmented if the intratumoral CD4⁺ to Treg ratio is increased after treatment as compared to before treatment. In some embodiments, the level, strength or extent of systemic immunity can be measured by determining the intratumoral Treg to CD45⁺ ratio, and the systemic immunity against recurring tumors is increased or augmented if the intratumoral Treg to CD45⁺ ratio is decreased after treatment as compared to before treatment. In some aspects, such increases or decreases can last for at or about 3, 4, 5, 6, 7, 8, 9, 10, 11, 12, 13, 14 days or 3, 4, 5, 6, 7 or 8 weeks or longer.

In some aspects, the level, strength or extent of systemic immunity can be measured by a CTL activity assay using splenocytes or peripheral blood cells or bone marrow cells or lymph node cells. In some embodiments, the cells are collected from the subject between day 4 and day 28 after illumination of the first tumor in the subject.

In some aspects, the level, strength or extent of systemic immunity can be measured by an intratumoral T cell exhaustion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of systemic immunity can be measured by an intratumoral effector T cell expansion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of systemic immunity can be measured by a T cell receptor diversity assay using T cells collected from the first tumor or a metastatic tumor cells mass or an invasive tumor cell mass or the peripheral circulation. In some embodiments, the cells are collected from the subject between day 4 and day 28 after illumination of the first tumor in the subject.

In some aspects, the level, strength or extent of systemic immunity can be measured by determining the presence, number or frequency of regulatory T cells (Tregs) in the tumor and/or the ratio of intratumoral Treg cells to intratumoral CD8+ T cells or intratumoral CD4+ T cells from the first tumor or a metastatic tumor cell mass or an invasive tumor cell mass. In some embodiments, the cells are collected from the subject between day 4 and day 28 after illumination of the first tumor in the subject.

In some embodiments, any of the above assays can be used in combination.

### III. Compositions for Use According to the Methods Herein

In some aspects, provided are compositions and combinations, for example, comprising a conjugate comprising a phthalocyanine dye linked to a targeting molecule, such as an antibody or an antigen-binding fragment thereof, that binds to CD25 protein; and an immune checkpoint inhibitor, such as an anti-PD-1 antibody. In some aspects, provided are compositions and combinations for use in a method of treatment or in a treatment regimen in accordance with the provided methods and uses, or in the manufacture of a medicament for the treatment of a cancer or a tumor. In some aspects, provided are compositions and combinations for use in accordance with the provided methods and uses.

The methods and uses provided herein employ a conjugate including a targeting molecule that binds to CD25, such as an anti-CD25 antibody or an antigen-binding fragment that binds to, such as specifically binds to, CD25. CD25 can be expressed on activated T cells, including CD8+ cells, CD4+FoxP3+ regulatory T cells, activated B cells, some thymocytes, myeloid precursors, and oligodendrocytes. CD25 is also known as interleukin 2 receptor alpha chain (IL2RA), IDDM10, IL2R, TCGFR, p55 or IMD41.

In some embodiments, the targeting molecule can be an anti-CD25 antibody such basiliximab (Simulect®), daclizumab, PC61, or antigen-binding fragment of an anti-CD25 antibody such basiliximab (Simulect®), daclizumab, or PC61. In some of any embodiments, the anti-CD25 antibody is daclizumab. In some embodiments, the anti-CD25 antibody is a basiliximab comprising an Fc region.

In some of any embodiments, the targeting molecule is an antibody or antigen-binding fragment thereof or includes an antigen-binding fragment. In some of any embodiments, the antibody is an anti-CD25 antibody. In some of any embodiments, the anti-CD25 antibody includes a functional Fc region. In some of any embodiments, the anti-CD25 antibody includes a full length Fc region. In some embodiments, the anti-CD25 antibody is an antibody fragment. In some of any of the embodiments, the anti-CD25 antibody is basiliximab or daclizumab, or biosimilar, interchangeable, biobetter, copy biological or biogeneric thereof. In some of any embodiments, the anti-CD25 antibody is basiliximab. In some of any of the embodiments, the anti-CD25 antibody is basiliximab comprising an Fc region. In some of any of the embodiments, the anti-CD25 antibody is basiliximab comprising an Fc region that is engineered to exhibit antibody-dependent cellular cytotoxicity (ADCC) activity or exhibit enhanced ADCC activity. In some of any of the embodiments, the anti-CD25 antibody is basiliximab comprising a full length Fc region. In some of any of the embodiments, the anti-CD25 antibody is basiliximab comprising an Fc region that is engineered to exhibit ADCC activity or exhibit enhanced ADCC activity. In some of any of the embodiments, the anti-CD25 antibody is basiliximab comprising an Fc region that does not exhibit ADCC activity or exhibit reduced ADCC activity. In some of any of the embodiments, the anti-CD25 antibody is daclizumab. In some of any of the embodiments, the anti-CD25 antibody is daclizumab comprising an Fc region. In some of any of the embodiments, the anti-CD25 antibody is daclizumab comprising a functional Fc region. In some of any of the embodiments, the anti-CD25 antibody is daclizumab comprising a full length Fc region. In some embodiments, the targeting molecule can be an antibody or antibody fragment that includes the “complementarity-determining regions” or “CDRs” of an anti-CD25 antibody, such as any of the described antibodies or antigen-binding fragment thereof. The CDRs are typically responsible for binding to an epitope of an antigen. The CDRs of each chain are typically referred to as CDR1, CDR2, and CDR3, numbered sequentially starting from the N-terminus, and are also generally identified by the chain in which the particular CDR is located. Thus, a heavy chain variable region (VH) CDR3 is located in the variable domain of the heavy chain of the antibody in which it is found, whereas a light chain variable region (VL) CDR1 is the CDR1 from the variable domain of the light chain of the antibody in which it is found. Antibodies with different specificities, such as different combining sites for different antigens, have different CDRs. Although it is the CDRs that vary from antibody to antibody, only a limited number of amino acid positions within the CDRs are directly involved in antigen binding. These positions within the CDRs are called specificity determining residues (SDRs). In some embodiments, the targeting molecule includes CDRs from basiliximab (Simulect®), daclizumab or PC61. In some embodiments, the targeting molecule is basiliximab (Simulect®). In some embodiments, the antibody of the conjugate is a biosimilar, interchangeable or biobetter of any of the anti-CD25 antibody described herein, e.g., basiliximab (Simulect®), or an antigen-binding fragment thereof. Such antibodies also include copy biologicals and biogenerics of any of the anti-CD25 antibody described herein, e.g., basiliximab (Simulect®), or an antigen-binding fragment thereof.

In some embodiments of the methods and uses provided herein, an anti-CD25 antibody comprises a functional Fc region. In some embodiments of the methods and uses provided herein, an anti-CD25 antibody comprises a full-length Fc region.

The conjugates used in the methods and uses provided herein include a phthalocyanine dye. In some embodiments of the methods and uses provided herein, a phthalocyanine dye is a phthalocyanine dye with a silicon coordinating metal (Si-phthalocyanine dye). In some embodiments, the phthalocyanine dye comprises the formula:

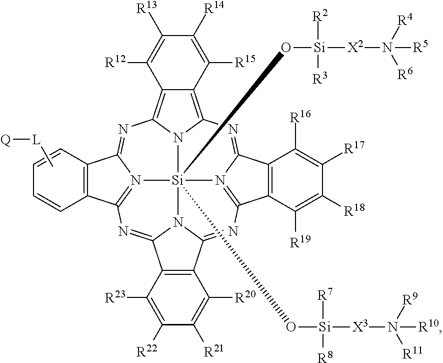

wherein:

L is a linker;

Q is a reactive group for attachment of the dye to the targeting molecule;

R², R³, R⁷, and R⁸ are each independently selected from among optionally substituted alkyl and optionally substituted aryl;

R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water-soluble group;

R¹², R¹³, R¹⁴, R¹⁵, R¹⁶, R¹⁷, R¹⁸, R¹⁹, R²⁰, R²¹, R²² and R²³ are each independently selected from among hydrogen, halogen, optionally substituted alkylthio, optionally substituted alkylamino and optionally substituted alkoxy; and

X² and X³ are each independently C₁-C₁₀ alkylene, optionally interrupted by a heteroatom.

In some embodiments, the phthalocyanine dye comprises the formula:

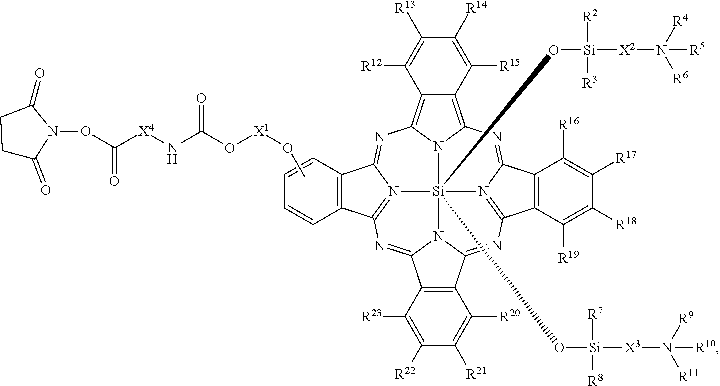

wherein:

X¹ and X⁴ are each independently a C₁-C₁₀ alkylene optionally interrupted by a heteroatom;

R², R³, R⁷, and R⁸ are each independently selected from optionally substituted alkyl and optionally substituted aryl;

R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water-soluble group; and

R¹⁶, R¹⁷, R¹⁸ and R¹⁹ are each independently selected from among hydrogen, halogen, optionally substituted alkylthio, optionally substituted alkylamino and optionally substituted alkoxy.

In some embodiments of the methods and uses provided herein, a Si-phthalocyanine dye is IRDye 700DX (IR700). In some embodiments, the phthalocyanine dye containing the reactive group is IR700 NHS ester, such as IRDye 700DX NHS ester (LiCor 929-70010, 929-70011). In some embodiments, the dye is a compound having the following formula:

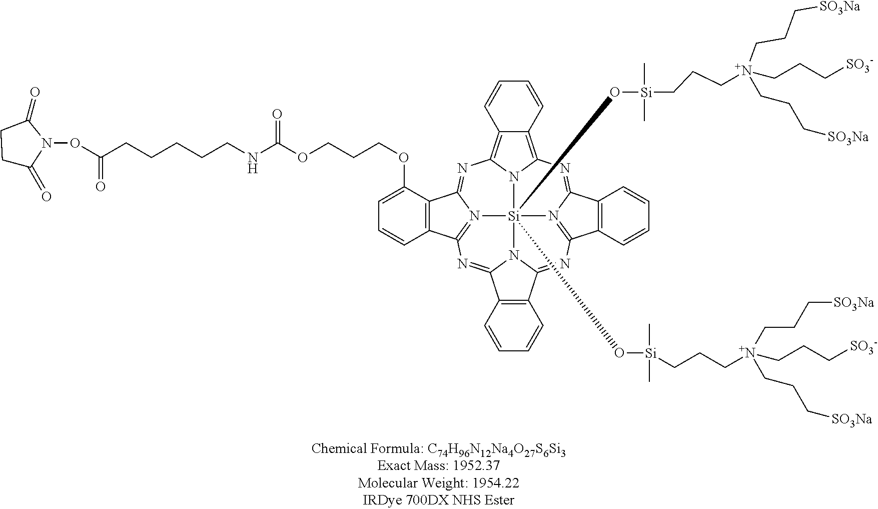

For purposes herein, the term “IR700,” “IRDye 700” or “IRDye 700DX” includes the above formula when the dye is conjugated such as to an antibody, e.g. via a reactive group.

In some embodiments the compositions for use with the methods and uses provided herein include a conjugate comprising a Si-phthalocyanine dye linked to targeting molecule, wherein the targeting molecule binds to CD25. In some embodiments, the composition is an anti-CD25-Si-phthalocyanine dye conjugate. In some embodiments, the composition is an anti-CD25-IR700 conjugate. In some embodiments, the composition is an anti-CD25-IR700 conjugate, where the anti-CD25 portion is basiliximab. In some embodiments, the composition is an anti-CD25-IR700 conjugate, where the anti-CD25 portion is basiliximab containing a functional Fc region. In some embodiments, the composition is an anti-CD25-IR700 conjugate, where the anti-CD25 portion is basiliximab containing an Fc region, such as a full-length Fc region. In some embodiments, the composition is an anti-CD25-IR700 conjugate, where the anti-CD25 portion is basiliximab containing an Fc region, such as an Fc region that is engineered to exhibit antibody-dependent cellular cytotoxicity (ADCC) activity or exhibit enhanced ADCC activity. In some embodiments, the composition is an anti-CD25-IR700 conjugate, where the anti-CD25 portion is daclizumab.

### IV. Checkpoint Inhibitor Combination Therapy

The methods and uses provided herein can include the administration of an immune checkpoint inhibitor prior to, concurrent with or subsequent to the administration of the conjugate. For example, the methods can include administering one or more doses of an immune checkpoint inhibitor, administering a conjugate comprising a phthalocyanine dye linked to targeting molecule, wherein the targeting molecule binds to CD25, and after administration of the conjugate, illuminating a first tumor and, optionally one or more additional tumors. The methods can include first administering a conjugate comprising a phthalocyanine dye linked to targeting molecule, wherein the targeting molecule binds to CD25, and after administration of the conjugate, illuminating a first tumor and, optionally one or more additional tumors, and then administering an immune checkpoint inhibitor subsequent either to administration of the conjugate or subsequent to the illumination (e.g., irradiation) step. The methods can also include the administration of an immune checkpoint inhibitor concurrently with administration of the conjugate.

In some embodiments, the immune checkpoint inhibitor is selected from a PD-1 inhibitor, a PD-L1 inhibitor, a CTLA-4 inhibitor, or a combination thereof. In some embodiments, the immune checkpoint inhibitor is selected from an antibody or antigen-binding fragment that binds to PD-1, an antibody or antigen-binding fragment that binds to PD-L1 or an antibody or antigen-binding fragment that binds to CTLA-4, or a combination thereof.

In some embodiments of the methods and uses provided herein, the immune checkpoint inhibitor is an anti-PD-1 antibody. In some embodiments, an anti-PD-1 antibody for use with the methods include, but are not limited to, pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-IIOA, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, TSR-042 (ANB011), and any combination thereof.

In some embodiments of the methods and uses provided herein, the immune checkpoint inhibitor is an anti-PD-L1 antibody. Anti-PD-L1 antibodies that can be used in the methods and uses provided herein include, but are not limited to, atezolizumab (MPDL3280A, Tecentriq, RG7446), avelumab (BAVENCIO, MSB0010718C; M7824), durvalumab (MEDI4736, IMFINZI), LDP, NM-01, STI-3031 (IMC-001; STI-A1015), KN035, LY3300054, M7824 (MSB0011359C), BMS-936559, MSB2311, BCD-135, BGB-A333, CBT-502 (TQB-2450), cosibelimab (CK-301), CS1001 (WPB3155), FAZ053, MDX-1105, SHR-1316 (HTI-1088), TG-1501, ZKAB001 (STI-A1014), INBRX-105, MCLA-145, KN046, LY3415244, REGN3504, HLX20, and any combination thereof.

In some embodiments of the methods and uses provided herein, the immune checkpoint inhibitor is an anti-CTLA-4 antibody. Exemplary anti-CTLA-4 antibodies are ipilimumab (YERVOY), tremelimumab (ticilimumab, CP-675,206), AGEN1181, AGEN1884, ADU-1064, BCD-145, BCD-217, ADG116, AK104, ATOR-1015, BMS-986218, KN046, MGD019, MK-1308, REGN4659, XmAb20717, XmAb22841, and any combination thereof.

In some embodiments of the methods and uses provided herein, the immune checkpoint inhibitor is selected from an antibody or antigen-binding fragment that binds to PD-1, PD-L1, or CTLA-4, and the conjugate is an anti-CD25-IR700 conjugate. In some embodiments of the methods and uses provided herein, the immune checkpoint inhibitor is antibody or antigen-binding fragment that binds to PD-1 and the conjugate is an anti-CD25-IR700 conjugate, where the anti-CD25 portion of the conjugate is or is derived from basiliximab. In some embodiments of the methods and uses provided herein, the immune checkpoint inhibitor is antibody or antigen-binding fragment that binds to PD-1 and the conjugate is an anti-CD25-IR700 conjugate, where the anti-CD25 portion of the conjugate is or is derived from basiliximab, and the antibody portion of the conjugate includes a functional Fc region. In some embodiments of the method, the conjugate is an anti-CD25-IR700 conjugate, where the anti-CD25 portion of the conjugate basiliximab, that includes a functional Fc region. In some embodiments of the method, the conjugate is an anti-CD25-IR700 conjugate, where the anti-CD25 portion of the conjugate basiliximab, that includes a functional Fc region, and the anti-PD-1 antibody is pembrolizumab (MK-3475, KEYTRUDA), nivolumab (OPDIVO), or cemiplimab (LIBTAYO).

In some embodiments of the methods and uses provided herein, an immune checkpoint inhibitor can be administered to a subject having cancer prior to the administration of anti-CD25-IR700 conjugate, concurrently to the administration of anti-CD25-IR700 conjugate, after the administration of anti-CD25-IR700 conjugate or any combination thereof.

In some embodiments of the methods and uses provided herein, an immune checkpoint inhibitor is administered one time, two times, three times, four times, five times, six times, seven times, eight times, nine times, 10 times, or more than 10 times to a subject.

In some embodiments, an immune checkpoint inhibitor is administered to a subject once, twice three times, four times, five times or more than five times prior to the administration of the conjugate. In some embodiments, an immune checkpoint inhibitor is administered to a subject at or about 12 hours, 24 hours, 48 hours, 96 hours, one week, two weeks, three weeks or four weeks prior to the administration of the conjugate. In some embodiments, an immune checkpoint inhibitor is administered to a subject less than 1-4 weeks or less than 1-3 weeks or 1-2 weeks prior to administration of the conjugate.

In some embodiments, an immune checkpoint inhibitor is administered to a subject once, twice three times, four times, five times or more than five times after to the administration of the conjugate. In some embodiments, an immune checkpoint inhibitor is administered to a subject prior to administration of the conjugate and after administration of the conjugate.

In some embodiments of the methods and uses provided herein, an anti-CD25 conjugate is administered to a subject once or more than once. In some embodiments, the conjugate is administered one time, two times, three times, four times, five times, six times, seven times, eight times, nine times, 10 times, or more than 10 times to the subject. In some embodiments, the first tumor(s) is illuminated after each administration of the conjugate, such as within 24 hours±4 hours after each administration of the conjugate. In some embodiments, the conjugate is administered more than once if a residual lesion remains in the subject, such as a first tumor or one or more additional tumors, residual cells or masses from a primary tumor, invasive cancer cells or metastatic tumor cells. In some embodiments, the dosing of the conjugate is repeated if a residual lesion remains at a time that is more than 2 weeks, 3 weeks, 4 weeks, 1 month, 2 months, 3 months, 4, months, 5 months, 6 months, 1 year or more than 1 year after the prior administration of the conjugate.

In some embodiments of the methods and uses provided herein, an anti-CD25 conjugate, such as an anti-CD25-IR700 conjugate is administered and the methods also include administration of an immune checkpoint inhibitor, wherein the dual administration of results in an enhanced effects, such as an additional, additive, or synergistic effect. Additional, additive, or synergistic effect refers to an effect that is more than the effect of either monotherapy alone (i.e. administration of only the conjugate, administration of only the conjugate followed by illumination, or administration of only the checkpoint inhibitor to a subject). For example, the administration of an anti-CD25-IR700 conjugate and administration of a checkpoint inhibitor, such as an anti-PD-1 antibody, produces an effect that is greater than if only the anti-CD25-IR700 conjugate or only the checkpoint inhibitor is administered, or if only the anti-CD25-IR700 conjugate is administered followed by illumination. In some aspects, the provided methods and uses results in an additional, additive, or synergistic anti-tumor response; for example, the growth and/or increase in volume, dimension(s), or mass of one or both of the first tumor and the number of cells in the secondary population in the subject is inhibited to a greater degree or extent as compared with administration of only the conjugate, only the conjugate followed by illumination, and/or only the anti-PD-1 antibody. In some aspects of the provided methods or uses, the growth and/or increase in volume, dimension(s), or mass of one or both of the first tumor and the volume, dimension(s), or cell number of the secondary population in the subject is inhibited to a greater degree as compared with administration of only the conjugate followed by illumination and as compared with administration of only the anti-PD-1 antibody. In some embodiments, the inhibition includes one or more of: a less than 20% increase in tumor volume, tumor dimensions or tumor mass; no change in tumor volume, dimension, or mass (i.e., halted tumor growth or progression); or a reduction in tumor volume, tumor dimensions or tumor mass, or a reduction in the number of tumor cells. In some embodiments, the inhibition includes one or more of: a less than 20% increase in the number of tumor cells. In some embodiments, the methods produce an enhanced response, such as a more complete response, a more durable response or longer lasting response.

In some embodiments, the methods produce an enhanced effect, such as an additional effect, an additive effect, or a synergistic effect on a first tumor including one or more of: inhibition, reduction or elimination of tumor growth, reduction in tumor volume, dimension(s), or mass, or an increase in numbers of subjects with a complete response, and any combinations thereof. In some embodiments, the methods produce an enhanced effect, such as an additional effect, an additive effect, or a synergistic effect, on invasive tumor cells including one or more of: inhibition, reduction or elimination of tumor cell growth, reduction in number or volume, dimension(s), or mass of invasive tumor cells, and any combinations thereof, including combinations with effects on one or more illuminated tumors, such as the first tumor. In some embodiments, the methods produce an enhanced effect, such as an additional effect, an additive effect, or a synergistic effect, on metastatic tumor cells including one or more of: inhibition, reduction or elimination of metastatic tumor cell growth, reduction in number or volume, dimension(s), or mass of metastatic tumor cells, and any combinations thereof, including combinations with effects on one or more illuminated tumors. In some embodiments, the synergistic effect is achieved on the tumor exposed directly to the illumination (i.e., illumination or irradiation with the selected wavelength of light). In some embodiments, the enhanced effect is achieved on tumor cells that were not illuminated, such as where a first tumor is illuminated, and a synergistic effect is achieved on metastatic tumor cells or invasive tumor cells that were not illuminated, such as metastatic tumor cells or invasive tumor cells located distal to the illuminated tumor that were not illuminated.

In some embodiments, the methods produce a synergistic effect on the increase or augmentation of systemic immunity. In some embodiments, the methods produce a synergistic effect on any of the measures of systemic immunity as described herein or known, such as those described in Section II herein, for example, the number of intratumoral CD8⁺ T lymphocytes, the ratio of CD8⁺ T lymphocytes to regulatory T cells (T_{regs}), intratumoral T lymphocyte exhaustion (e.g., the percentage of CD3⁺CD8⁺ cells that express PD-1 and/or CTLA4 markers), the number or percentage of intratumoral activated CD8⁺ T lymphocytes (e.g., Ki67⁺ or CD69⁺CD8 cells as a percentage of CD45⁺ cells), the expansion of cytotoxic intratumoral T lymphocytes (e.g., the percentage of CD3⁺CD8⁺ cells that do not express PD-1 and/or CTLA4 markers), based on the splenocyte cytotoxicity against tumor cells, or any or all of combination thereof. In some aspects, intratumoral CD8⁺ T lymphocytes include CD3⁺CD8⁺ cells, intratumoral exhausted T lymphocytes include PD-1⁺CTLA-4⁺CD3⁺CD8⁺ cells, activated intratumoral CD8⁺ T lymphocytes include CD3⁺CD8⁺Ki67⁺ and/or CD3⁺CD8⁺CD69⁺ cells, expansion of cytotoxic T lymphocytes include PD-1⁻CTLA-4⁻CD3⁺CD8⁺ cells.

In some embodiments, the methods include administration of an anti-CD25-IR700 conjugate and an immune checkpoint inhibitor to achieve a synergistic effect. In some embodiments, the methods include administration of an anti-CD25-IR700 conjugate and an anti-PD-1 antibody, to achieve an enhanced effect, such as a synergistic effect or an additive effect. In some embodiments, the anti-CD25-IR700 conjugate comprises basiliximab with a function Fc region and the immune checkpoint inhibitor is an anti-PD-1 antibody such as nivolumab, and an enhanced effect, such as an additional, additive, or synergistic effect, is seen as a reduction of tumor growth, reduction of tumor volume, reduction in tumor dimension, reduction in tumor mass, or a complete response, in an illuminated tumor, and/or a reduction in the growth, number, volume, dimension, or mass of non-illuminated tumor cells in a secondary population of tumor cells (such as invasive tumor cells or metastatic tumor cells). In some embodiments, administration of an anti-CD25-IR700 conjugate and illumination results in a reduction of the number of intratumoral Tregs and/or an increase of intratumoral CD8+ to Treg ratio or intratumoral CD4+ to Treg ratio. In some embodiments, a combination treatment with an anti-PD-1 antibody can result in a synergistic effect with respect to a reduction of the number of intratumoral T_{regs} and/or an increase of intratumoral CD8+ to Treg ratio or intratumoral CD4+ to Treg ratio.

### V. Additional Therapeutic Agent

In some aspects, the provided methods and uses involve administration of an additional therapeutic agent or anti-cancer treatment. In some aspects, the additional therapeutic agent is an immune modulating agent. In some aspects, the additional therapeutic agent is an anti-cancer treatment.

In some embodiments, the immune-modulating agent is cytokine. In some embodiments, the immune modulating agent is a cytokine or is an agent that induces increased expression of a cytokine in the tumor microenvironment. In some aspects, “cytokine” refers to a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immune modulating agent is a cytokine and the cytokine is IL-4, TNF-a, GM-CSF or IL-2.

In some embodiments, the immune modulating agent is selected from among GM-CSF, CpG-ODN (CpG oligodeoxynucleotides), lipopolysaccharide (LPS), monophosphoryl lipid A (MPL), alum, recombinant Leishmania polyprotein, imiquimod, MF59, poly I:C, poly A:U, type 1 IFN, Pam3Cys, Pam2Cys, complete freund's adjuvant (CFA), alpha-galactosylceramide, RC-529, MDF2P, Loxoribine, anti-CD40 agonist, SIRPa antagonist, AS04, AS03, Flagellin, Resiquimod, DAP (diaminopimelic acid), MDP (muramyl dipeptide) and CAF01 (cationic adjuvant formulation-01). In some embodiments, the immune modulating agent is a Toll-like receptor (TLR) agonist, an adjuvant or a cytokine. In some embodiments, the immune modulating agent is a TLR agonist and the TLR agonist is TLR agonist is a TLR4 agonist, a TLR7 agonist, a TLR8 agonist, or a TLR9 agonist. In some embodiments, the TLR agonist is selected from among triacylated lipoprotein, diacylated lipopeptide, lipoteichoic acid, peptidoglycan, zymosan, Pam3CSK4, dsRNA, polyLC, Poly G10, Poly G3, CpG, 3M003, flagellin, lipopolysaccharide (LPS) Leishmania homolog of eukaryotic ribosomal elongation and initiation factor 4a (LeIF), MED 19197, SD-101, and imidazoquinoline TLR agonists.

In some embodiments, the immune modulating agent can contain one or more interleukins or other cytokines. For example, the interleukin can include leukocyte interleukin injection (Multikine), which is a combination of natural cytokines.

In some embodiments, the immune modulating agent is a Toll-like receptor (TLR) agonist. In some embodiments, such agonists can include a TLR4 agonist, a TLR8 agonist, or a TLR9 agonist. Such an agonist may be selected from peptidoglycan, polyLC, CpG, 3M003, flagellin, and Leishmania homolog of eukaryotic ribosomal elongation and initiation factor 4a (LeIF).

In some embodiments, the immune modulating agent can be one that enhances the immunogenicity of tumor cells such as patupilone (epothilone B), epidermal-growth factor receptor (EGFR)-targeting monoclonal antibody 7A7.27, histone deacetylase inhibitors (e.g., vorinostat, romidepsin, panobinostat, belinostat, and entinostat), the n3-polyunsaturated fatty acid docosahexaenoic acid, proteasome inhibitors (e.g., bortezomib), shikonin (the major constituent of the root of Lithospermum erythrorhizon,) and oncolytic viruses, such as TVec (talimogene laherparepvec). In some embodiments, the immune modulating agent activates immunogenic cell death of the cancer or tumor, such as antrhacyclins (doxorubicin, mitoxantron), BK channel agonists, bortezomib, botrtezomib plus mitocycin C plus hTert-Ad, Cardiac glycosides plus non-ICD inducers, cyclophosphamide, GADD34/PP1 inhibitors plus mitomycin, LV-tSMAC, and oxaliplatin. In some embodiments, the immune modulating agent can be an epigenetic therapy, such as DNA methyltransferase inhibitors (e.g., Decitabine, 5-aza-2′-deoxycytidine).

For example, in some embodiments, the immune modulating agent can be a DNA methyltransferase inhibitor, which can regulate expression of tumor associated antigens (TAA). TAAs are antigenic substances produced in tumor cells which triggers an immune response. TAAs are often down-regulated by DNA methylation in tumors to escape the immune system. Reversal of DNA methylation restores TAA expression, increasing the immunogencity of tumor cells. For example, demethylating agents such as decitabine (5-aza-2′-deoxycytidine) can upregulate expression of TAAs in tumor cells and increase immune recognition of the cancerous cells. Photoimmunotherapy would further expose TAAs to the immune system by disrupting cells.

In some embodiments, the additional therapeutic agent is an agent or compound, used in anti-cancer treatment, such as anti-cancer agents. These include any agents, that can alleviate, reduce, ameliorate, prevent, or place or maintain in a state of remission of clinical symptoms or diagnostic markers associated with tumors and cancer, and can be used in combinations and compositions provided herein. In some embodiments, the anti-cancer agent is one whose therapeutic effect is generally associated with penetration or delivery of the anti-cancer agent into the tumor microenvironment or tumor space. In some embodiments, the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor or an antibody or antigen-binding antibody fragment thereof. In some embodiments, the anti-cancer agent is a peptide, protein or small molecule drug.

In some embodiments, the anti-cancer agent is 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin or auroropyrimidine.

In some embodiments, the anti-cancer agent is an alkylating agent. Alkylating agents are compounds that directly damage DNA by forming covalent bonds with nucleic acids and inhibiting DNA synthesis. Exemplary alkylating agents include, but are not limited to, mechlorethamine, cyclophosphamide, ifosamide, melphalan, chlorambucil, busulfan, and thiotepa as well as nitrosurea alkylating agents such as carmustine and lomustine.

In some embodiments, the anti-cancer agent is an antibody or antigen-binding antibody fragment.

In some embodiments, the anti-cancer agent is a platinum drug. Platinum drugs bind to and cause crosslinking of DNA, which ultimately triggers apoptosis. Exemplary platinum drugs include, but are not limited to, cisplatin, carboplatin, oxaliplatin, satraplatin, picoplatin, nedaplatin, triplatin, and lipoplatin.

In some embodiments, the anti-cancer agent is an antimetabolite. Antimetabolites interfere with DNA and RNA growth by substituting for the normal building blocks of RNA and DNA. These agents damage cells during the S phase, when the cell's chromosomes are being copied. In some cases, antimetabolites can be used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®).

In some embodiments, the anti-cancer agent is an anti-tumor antibiotic. Anti-tumor antibiotics work by altering the DNA inside cancer cells to keep them from growing and multiplying. Anthracyclines are anti-tumor antibiotics that interfere with enzymes involved in DNA replication. These drugs generally work in all phases of the cell cycle. They can be widely used for a variety of cancers. Exemplary anthracyclines include, but are not limited to, daunorubicin, doxorubicin, epirubicin, and idarubicin. Other anti-tumor antibiotics include actinomycin-D, bleomycin, mitomycin-C, and mitoxantrone.

In some embodiments, the anti-cancer agent is a topoisomerase inhibitor. These drugs interfere with enzymes called topoisomerases, which help separate the strands of DNA so they can be copied during the S phase. Topoisomerase inhibitors can be used to treat certain leukemias, as well as lung, ovarian, gastrointestinal, and other cancers. Exemplary toposiomerase inhibitors include, but are not limited to, doxorubicin, topotecan, irinotecan (CPT-11), etoposide (VP-16), teniposide, and mitoxantrone.

In some embodiments, the anti-cancer agent is a mitotic inhibitor. Mitotic inhibitors are often plant alkaloids and other compounds derived from natural plant products. They work by stopping mitosis in the M phase of the cell cycle but, in some cases, can damage cells in all phases by keeping enzymes from making proteins needed for cell reproduction. Exemplary mitotic inhibitors include, but are not limited to, paclitaxel (Taxol®), docetaxel (Taxotere®), ixabepilone (Ixempra®), vinblastine (Velban®), vincristine (Oncovin®), vinorelbine (Navelbine®), and estramustine (Emcyt®).

In some embodiments, the anti-cancer agent is a corticosteroid. Corticosteroids, often simply called steroids, are natural hormones and hormone-like drugs that are useful in the treatment of many types of cancer. Corticosteroids can also be used before chemotherapy to help prevent allergic reactions as well as during and after chemotherapy to help prevent nausea and vomiting. Exemplary corticosteroids include, but are not limited to, prednisone, methylprednisolone (Solumedrol®), and dexamethasone (Decadron®).

In some embodiments, the anti-cancer agent is another type of chemotherapy drug, such as a proteosome inhibitor, a kinase inhibitor, or a histone-deacetylase inhibitor. In other embodiments, the anti-cancer agent is a biologic such as an antibody used in cancer therapy.

### VI. Devices for Use with the Methods and Compositions

In some aspects, devices that can be used with the methods and compositions herein include light diffusing devices that provide illumination at a wavelength (or wavelengths) of light wavelength suitable for use with the dye conjugate composition, such as a phthalocyanine dye conjugate (e.g., an anti-CD25-IR700 conjugate such as those described herein). Illumination devices can include a light source (for example a laser), and a means of conveying the light to the area of interest (for example, one or more fibers to illuminate an isolated area of a subject or an isolated lesion or tumor).

In some embodiments of the methods and uses provided herein, illumination is carried out using cylindrical diffusing fibers that includes a diffuser length of 0.5 cm to 10 cm and spaced 1.8±0.2 cm apart. In some embodiments, the light illumination dose is from or from about 20 J/cm fiber length to about 500 J/cm fiber length. In some embodiments, the lesion is a tumor that is an interstitial tumor and illumination is carried out using cylindrical diffusing fibers. In some embodiments, the tumor is greater than 10 mm deep or is a subcutaneous tumor.

In some embodiments, the provided methods include illuminating an interstitial tumor in a subject with cylindrical diffusing fibers that includes a diffuser length of 0.5 cm to 10 cm and spaced 1.8±0.2 cm apart with a light dose of or about 100 J/cm fiber length or with a fluence rate of or about 400 mW/cm. In some embodiments, the tumor is greater than 10 mm deep or is a subcutaneous tumor. In some embodiments, the cylindrical diffusing fibers are placed in a catheter positioned in the tumor 1.8±0.2 cm apart. In some embodiments, the catheter is optically transparent.

In some embodiments, the lesion is a tumor that is a superficial tumor. In some embodiments, the tumor is less than 10 mm thick. In some embodiments, illumination is carried out using a microlens-tipped fiber for surface illumination. In some embodiments, the light illumination dose is from or from about 5 J/cm² to about 200 J/cm².

In some embodiments, the provided methods include illuminating a superficial tumor in a subject with a microlens-tipped fiber for surface illumination with a light dose of from or from about 5 J/cm² to about 200 J/cm². In some embodiments, the light illumination dose is or is about 50 J/cm².

In some embodiments, the illumination (also referred to herein as illumination) employ a device with “top hat” irradiance distribution profile, such as those described in WO2018/080952 and US20180239074.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “conjugate” refers to a targeting molecule linked directly or indirectly to a photoactivatable dye, such as those produced by chemical conjugates and those produced by any other methods. For example, a conjugate can refer to a phthalocyanine dye, such as an IR700 molecule, linked directly or indirectly to one or more targeting molecules, such as to a polypeptide binds to or targets to a cell surface protein. A targeting molecule can be a polypeptide, more than one polypeptide, an antibody or a chemical moiety.

As used herein an “anti-CD25 conjugate” refers to a conjugate having a targeting molecule that binds to CD25. An anti-CD25 conjugate can have a targeting molecule that is an antibody, antigen-binding fragment or other moiety that binds to CD25.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

“Specifically binds” refers to the ability of individual antibodies to specifically immunoreact with an antigen, such as a tumor-specific antigen, relative to binding to unrelated proteins, such as non-tumor proteins, for example β-actin. For example, a CD25-specific binding agent binds substantially only the CD25 protein in vitro or in vivo. As used herein, the term “tumor-specific binding agent” includes tumor-specific antibodies and other agents that bind substantially only to a tumor-specific protein in that preparation.

“Antibody-IR700 molecule” or “antibody-IR700 conjugate” refers to a molecule that includes both an antibody, such as a tumor-specific antibody, conjugated to IR700. In some examples the antibody is a humanized antibody (such as a humanized monoclonal antibody) that specifically binds to a surface protein on a cancer cell.

“Antigen” refers to a compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific peptide (such as one found on the surface of a cancer cell) or immunogenic fragment thereof.

“Immune checkpoint inhibitor” refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include PD-1/PD-L1 and CTLA-4/B7-1/B7-2. Some immune checkpoint inhibitors are used to treat cancer.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “combination therapy” refers to a treatment in which a subject is given two or more therapeutic agents, such as at least two or at least three therapeutic agents, for treating a single disease. In some embodiments, each therapy can result in an independent pharmaceutical effect, and together can result in an additive or synergistic pharmaceutical effect.

As used herein, “treating” a subject with a disease or condition means that the subject's symptoms are partially or totally alleviated or remain static following treatment. Hence treating encompasses prophylaxis, therapy and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### VIII. Exemplary Embodiments

Among the embodiments provided herein are:

1. A method for treating a cancer, comprising:

(a) administering to a subject having a cancer comprising a primary tumor and metastatic tumor cells, a conjugate comprising a phthalocyanine dye linked to a targeting molecule, wherein the targeting molecule binds to CD25,

(b) administering an immune checkpoint inhibitor to the subject, and

(c) after administering the conjugate, illuminating the primary tumor at a wavelength of at or about 600 nm to at or about 850 nm and at a dose of from at or about 25 J/cm² to at or about 400 J/cm² or from at or about 2 J/cm fiber length to at or about 500 J/cm fiber length,

wherein the growth and/or increase in volume of one or both of the primary tumor and metastatic tumor cells in the subject is inhibited.

2. The method of embodiment 1, wherein the metastatic tumor cells are not directly illuminated.

3. The method of embodiment 1 or 2, wherein the metastatic tumor cells are comprised in a solid tumor.

4. The method of any of embodiments 1-3, wherein the growth inhibition of the primary tumor, the metastatic tumor cells or both is synergistic as compared with administration of only one of the conjugate and the immune checkpoint inhibitor.

5. The method of any of embodiments 1-4, wherein the subject exhibits a complete response.

6. The method of any of embodiments 1-5, wherein the targeting molecule is an antibody or antigen-binding fragment thereof or comprises an antigen-binding fragment.

7. The method of embodiment 6, wherein the antibody is an anti-CD25 antibody.

8. The method of embodiment 7, wherein the anti-CD25 antibody comprises a functional Fc region.

9. The method of embodiment 7 or 8, wherein the anti-CD25 antibody is basiliximab or daclizumab, or biosimilar, interchangeable, biobetter, copy biological or biogeneric thereof.

10. The method of any of embodiments 7-9, wherein the anti-CD25 antibody is basiliximab.

11. The method of any of embodiments 1-10, wherein the immune checkpoint inhibitor is selected from the group consisting of a PD-1 inhibitor, a PD-L1 inhibitor or a CTLA-4 inhibitor.

12. The method of any of embodiments 1-11, wherein the immune checkpoint inhibitor is or comprises an anti-PD-1 antibody, an anti-PD-L1 antibody or an anti-CTLA-4 antibody, or an antigen-binding fragment thereof.

13. The method of embodiment 12, wherein the immune checkpoint inhibitor is or comprises an anti-PD-1 antibody or an antigen-binding fragment thereof.

14. The method of embodiment 12 or 13, wherein the anti-PD-1 antibody is selected from the group consisting of pembrolizumab (MK-3475, KEYTRUDA), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab.

15. The method of embodiment 12, wherein the anti-PD-L1 antibody is selected from the group consisting of atezolizumab (MPDL3280A, Tecentriq), avelumab (Bavencio), durvalumab (MEDI4736, Imfinzi), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

16. The method of embodiment 12, wherein the anti-CTLA-4 antibody is selected from the group consisting of ipilimumab (Yervoy), tremelimumab, AGEN1181, AGEN1884, ADU-1064, BCD-145, and BCD-217.

17. The method of any of embodiments 1-16, wherein the conjugate is administered to the subject before, concurrently, or after the administration of the immune checkpoint inhibitor.

18. The method of any of embodiments 1-17, wherein the immune checkpoint inhibitor is administered once, twice, three times, four times, five times, six times, seven times, eight times, nine times, 10 times, or more than 10 times to the subject.

19. The method of any of embodiments 1-18, wherein the immune checkpoint inhibitor is administered once, twice, three times, four times, five times, six times, seven times, eight times, nine times, or 10 times before the administration of the conjugate, and subsequently followed by the administration of the immune checkpoint inhibitor zero time, once, twice, three times, four times, five times, six times, seven times, eight times, nine times, 10 times, or more than 10 times to the subject after administration of the conjugate.

20. The method of any of embodiments 1-18, wherein the immune checkpoint inhibitor is administered concurrently with the administration of the conjugate, and subsequently followed by the administration of the immune checkpoint inhibitor zero time, once, twice, twice, three times, four times, five times, six times, seven times, eight times, nine times, 10 times, or more than 10 times to the subject after administration of the conjugate.

21. The method of any of embodiments 1-18, wherein the immune checkpoint inhibitor is administered once, twice, three times, four times, five times, six times, seven times, eight times, nine times, 10 times, or more than 10 times after the administration of the conjugate to the subject.

22. The method of any of embodiments 1-21, wherein the phthalocyanine dye is a Si-phthalocyanine dye.

23. The method of embodiment 22, wherein the Si-phthalocyanine dye is IR700.

24. The method of any of embodiments 1-23, wherein the illumination is carried out between 30 minutes and 96 hours after administering the conjugate.

25. The method of any of embodiments 1-24, wherein the illumination is carried out 24 hours±4 hours after administering the conjugate.

26. The method of any of embodiments 1-25, wherein the primary tumor is illuminated at a wavelength of 690±40 nm.

27. The method of any of embodiments 1-26, wherein the primary tumor is illuminated at a dose of or about of 50 J/cm² or 100 J/cm of fiber length.

28. The method of any of embodiments 1-27, wherein one or more of steps (a), (b) or (c) are repeated.

29. The method of any of embodiments 1-28, further comprising (d) administering an additional therapeutic agent or anti-cancer treatment.

30. The method of any of embodiments 1-29, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

31. The method of any of embodiments 1-30, wherein the metastatic tumor cells are located in one, two, three, or more than three tissues or organs that are different from the tissue or organ where the primary tumor is located.

32. A method for treating a cancer, comprising:

(1) administering to a subject having a cancer comprising a primary tumor and invasive tumor cells, a conjugate comprising a phthalocyanine dye linked to a targeting molecule, wherein the targeting molecule binds to CD25,

(2) administering an immune checkpoint inhibitor to the subject, and

(3) after administering the conjugate, irradiating the primary tumor at a wavelength of at or about 600 nm to at or about 850 nm and at a dose of from at or about 25 J/cm² to at or about 400 J/cm² or from at or about 2 J/cm fiber length to at or about 500 J/cm fiber length,

wherein the growth and/or increase in volume of one or both of the primary tumor and invasive tumor cells in the subject is inhibited.

33. The method of embodiment 32, wherein the invasive tumor cells are not directly illuminated.

34. The method of embodiment 32 or 33, wherein the invasive tumor cells are comprised in a solid tumor.

35. The method of any of embodiments 32-34, wherein the growth inhibition of the primary tumor, the invasive tumor cells or both is synergistic as compared with administration of only one of the conjugate and the immune checkpoint inhibitor.

36. The method of any of embodiments 32-35, wherein the subject exhibits a complete response.

37. The method of any of embodiments 32-36, wherein the targeting molecule is an antibody or antigen-binding fragment thereof or comprises an antigen-binding fragment.

38. The method of embodiment 37, wherein the antibody is an anti-CD25 antibody.

39. The method of embodiment 38, wherein the anti-CD25 antibody comprises a functional Fc region.

40. The method of embodiment 38 or 39, wherein the anti-CD25 antibody is basiliximab or daclizumab, or biosimilar, interchangeable, biobetter, copy biological or biogeneric thereof.

41. The method of any of embodiments 38-40, wherein the anti-CD25 antibody is basiliximab.

42. The method of any of embodiments 32-41, wherein the immune checkpoint inhibitor is selected from the group consisting of a PD-1 inhibitor, a PD-L1 inhibitor or a CTLA-4 inhibitor.

43. The method of any of embodiments 32-42, wherein the immune checkpoint inhibitor is or comprises an anti-PD-1 antibody, an anti-PD-L1 antibody or an anti-CTLA-4 antibody, or an antigen-binding fragment thereof.

44. The method of embodiment 43, wherein the immune checkpoint inhibitor is or comprises an anti-PD-1 antibody or an antigen-binding fragment thereof.

45. The method of embodiment 43 or 44, wherein the anti-PD-1 antibody is selected from the group consisting of pembrolizumab (MK-3475, KEYTRUDA), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb207l7, RO7121661, CX-188, and spartalizumab.

46. The method of embodiment 43, wherein the anti-PD-L1 antibody is selected from the group consisting of atezolizumab (MPDL3280A, Tecentriq), avelumab (Bavencio), durvalumab (MEDI4736, Imfinzi), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

47. The method of embodiment 43, wherein the anti-CTLA-4 antibody is selected from the group consisting of ipilimumab (Yervoy), tremelimumab, AGEN1181, AGEN1884, ADU-1064, BCD-145, and BCD-217.

48. The method of any of embodiments 32-47, wherein the conjugate is administered to the subject before, concurrently, or after the administration of the immune checkpoint inhibitor.

49. The method of any of embodiments 32-48, wherein the immune checkpoint inhibitor is administered once, twice, three times, four times, five times, six times, seven times, eight times, nine times, 10 times, or more than 10 times to the subject.

50. The method of any of embodiments 32-49, wherein the immune checkpoint inhibitor is administered once, twice, three times, four times, five times, six times, seven times, eight times, nine times, or 10 times before the administration of the conjugate, and subsequently followed by the administration of the immune checkpoint inhibitor zero time, once, twice, three times, four times, five times, six times, seven times, eight times, nine times, 10 times, or more than 10 times to the subject after administration of the conjugate.

51. The method of any of embodiments 32-49, wherein the immune checkpoint inhibitor is administered concurrently with the administration of the conjugate, and subsequently followed by the administration of the immune checkpoint inhibitor zero time, once, twice, twice, three times, four times, five times, six times, seven times, eight times, nine times, 10 times, or more than 10 times to the subject after administration of the conjugate.

52. The method of any of embodiments 32-49, wherein the immune checkpoint inhibitor is administered once, twice, three times, four times, five times, six times, seven times, eight times, nine times, 10 times, or more than 10 times after the administration of the conjugate to the subject.

53. The method of any of embodiments 32-52, wherein the phthalocyanine dye is a Si-phthalocyanine dye.

54. The method of embodiment 53, wherein the Si-phthalocyanine dye is IR700.

55. The method of any of embodiments 32-54, wherein the illumination is carried out between 30 minutes and 96 hours after administering the conjugate.

56. The method of any of embodiments 32-55, wherein the illumination is carried out 24 hours±4 hours after administering the conjugate.

57. The method of any of embodiments 32-56, wherein the primary tumor is illuminated at a wavelength of 690±40 nm.

58. The method of any of embodiments 32-57, wherein the primary tumor is illuminated at a dose of or about of 50 J/cm² or 100 J/cm of fiber length.

59. The method of any of embodiments 32-58, wherein one or more of steps (1), (2) or (3) are repeated.

60. The method of any of embodiments 32-59, wherein further comprising (4) administering an additional therapeutic agent or anti-cancer treatment.

61. The method of any of embodiments 32-60, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

62. A method for enhancing systemic immunity in a subject having a tumor, comprising:

(a) administering to a subject having a tumor a conjugate comprising a phthalocyanine dye linked to an anti-CD25 antibody,

(b) administering an immune checkpoint inhibitor to the subject, and

(c) after administering the conjugate, irradiating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length,

wherein systemic immunity of the subject is enhanced as compared to the systemic immunity of the subject prior to the administration of the conjugate and the immune checkpoint inhibitor.

63. The method of embodiment 62, wherein systemic immunity is measured by one or more of a cytotoxic T lymphocyte (CTL) activity assay, an intratumoral T cell exhaustion assay, an intratumoral effector T cell expansion assay, or a T cell receptor diversity assay.

64. The method of embodiment 62 or 63, wherein:

the tumor comprises a primary tumor and metastatic tumor cells, and wherein the primary tumor is illuminated and the metastatic tumor cells are not directly illuminated; or

the tumor comprises a primary tumor and invasive tumor cells, and wherein the primary tumor is illuminated and the invasive tumor cells are not directly illuminated.

65. The method of any of embodiments 62-64, wherein systemic immunity is measured by a CTL activity assay using splenocytes or peripheral blood cells or bone marrow cells or lymph node cells, optionally collected from the subject between day 4 and day 28 after illumination of the primary tumor in the subject.

66. The method of any of embodiments 62-64, wherein systemic immunity is measured by an intratumoral T cell exhaustion assay using T cells collected from the primary tumor or a metastatic tumor cell mass or an invasive tumor cell mass, optionally collected from the subject between day 4 and day 28 after illumination of the primary tumor in the subject.

67. The method of any of embodiments 62-64, wherein systemic immunity is measured by an intratumoral effector T cell expansion assay using T cells collected from the primary tumor or a metastatic tumor cell mass or an invasive tumor cell mass, optionally collected from the subject between day 4 and day 28 after illumination of the primary tumor in the subject.

68. The method of any of embodiments 62-64, wherein systemic immunity is measured by a T cell receptor diversity assay using T cells collected from the primary tumor or a metastatic tumor cell mass or an invasive tumor cell mass or the peripheral circulation, optionally collected from the subject between day 4 and day 28 after illumination of the primary tumor in the subject.

69. The method of any of embodiments 62-64, wherein systemic immunity is measured by determining the presence, number or frequency of regulatory T cells (Tregs) in the tumor and/or the ratio of intratumoral Treg cells to intratumoral CD8+ T cells or intratumoral CD4+ T cells from the primary tumor or a metastatic tumor cell mass or an invasive tumor cell mass, optionally collected from the subject between day 4 and day 28 after illumination of the primary tumor in the subject.

70. The method of any of embodiments 62-69, wherein the anti-CD25 antibody comprises a functional Fc region.

71. The method of any of embodiments 62-70, wherein the anti-CD25 antibody is basiliximab or daclizumab, or biosimilar, interchangeable, biobetter, copy biological or biogeneric thereof.

72. The method of any of embodiments 62-71, wherein the anti-CD25 antibody is basiliximab.

73. The method of any of embodiments 62-72, wherein the immune checkpoint inhibitor is or comprises an anti-PD-1 antibody, an anti-PD-L1 antibody or an anti-CTLA-4 antibody, or an antigen-binding fragment thereof.

74. The method of embodiments 73, wherein the immune checkpoint inhibitor is or comprises an anti-PD-1 antibody or an antigen-binding fragment thereof.

75. The method of embodiment 73 or 74, wherein the anti-PD-1 antibody is selected from the group consisting of pembrolizumab (MK-3475, KEYTRUDA), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab.

76. The method of embodiment 73, wherein the anti-PD-L1 antibody is selected from the group consisting of atezolizumab (MPDL3280A, Tecentriq), avelumab (Bavencio), durvalumab (MEDI4736, Imfinzi), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

77. The method of embodiment 73, wherein the anti-CTLA-4 antibody is selected from the group consisting of ipilimumab (Yervoy), tremelimumab, AGEN1181, AGEN1884, ADU-1064, BCD-145, and BCD-217.

78. The method of any of embodiments 62-77, wherein the conjugate is administered to the subject before, concurrently with, or after the administration of the immune checkpoint inhibitor.

79. The method of any of embodiments 62-78, wherein the immune checkpoint inhibitor is administered once, twice, three times, four times, five times, six times, seven times, eight times, nine times, 10 times, or more than 10 times to the subject.

80. The method of any of embodiments 62-79, wherein the phthalocyanine dye is a Si-phthalocyanine dye.

81. The method of embodiment 80, wherein the Si-phthalocyanine dye is IR700.

82. The method of any of embodiments 62-81, wherein the illumination is carried out between 30 minutes and 96 hours after administering the conjugate and immune checkpoint inhibitor.

83. The method of any of embodiments 62-82, wherein the illumination is carried out 24 hours±4 hours after administering the conjugate and immune checkpoint inhibitor.

84. The method of any of embodiments 62-83, wherein the tumor is illuminated at a wavelength of 690±40 nm.

85. The method of any of embodiments 62-84, wherein the tumor is illuminated at a dose of or about of 50 J/cm² or 100 J/cm of fiber length.

86. The method of any of embodiments 62-85, wherein one or more of steps (a), (b), (c) or (d) are repeated.

87. The method of any of embodiments 62-86, wherein the tumor is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

88. A method for generating a synergistic response in a subject, comprising

(1) administering to a subject having cancer comprising a primary tumor a conjugate comprising a phthalocyanine dye linked to a targeting molecule, wherein the targeting molecule binds to CD25,

(2) administering an immune checkpoint inhibitor to the subject, and

(3) after administering the conjugate, irradiating the primary tumor at a wavelength of at or about 600 nm to at or about 850 nm and at a dose of from at or about 25 J/cm² to at or about 400 J/cm² or from at or about 2 J/cm fiber length to at or about 500 J/cm fiber length,

wherein the growth and/or increase in volume of the primary tumor is synergistically reduced as compared to administration of only the conjugate or only the immune checkpoint inhibitor.

89. The method of embodiment 88, wherein the cancer further comprises metastatic tumor cells, and wherein the growth and/or increase in volume of the metastatic tumor cells is reduced as compared to administration of only the conjugate or only the immune checkpoint inhibitor.

90. The method of embodiment 89, wherein the reduction of growth or the increase in volume of the metastatic tumor cells is synergistic.

91. The method of embodiment 88 wherein the cancer further comprises invasive tumor cells, and wherein the growth and/or increase in volume of the invasive tumor cells is reduced as compared to administration of only the conjugate or only the immune checkpoint inhibitor.

92. The method of embodiment 91, wherein the reduction of growth or the increase in volume of the invasive tumor cells is synergistic.

93. The method of any of embodiments 88-84, wherein the targeting molecule is an antibody or antigen-binding fragment thereof or comprises an antigen-binding fragment.

94. The method of any of embodiments 88-93, wherein the antibody is an anti-CD25 antibody.

95. The method of embodiment 94, wherein the anti-CD25 antibody comprises a functional Fc region.

96. The method of any of embodiments 88-95, wherein the anti-CD25 antibody is basiliximab or daclizumab, or biosimilar, interchangeable, biobetter, copy biological or biogeneric thereof.

97. The method of any of embodiments 88-96, wherein the anti-CD25 antibody is basiliximab.

98. The method of any of embodiments 88-97, wherein the immune checkpoint inhibitor is selected from the group consisting of a PD-1 inhibitor, a PD-L1 inhibitor or a CTLA-4 inhibitor.

99. The method of any of embodiments 88-98, wherein the immune checkpoint inhibitor is or comprises an anti-PD-1 antibody, an anti-PD-L1 antibody or an anti-CTLA-4 antibody, or an antigen-binding fragment thereof.

100. The method of embodiment 99, wherein the immune checkpoint inhibitor is or comprises an anti-PD-1 antibody or an antigen-binding fragment thereof.

101. The method of embodiment 99 or 100, wherein the anti-PD-1 antibody is selected from the group consisting of pembrolizumab (MK-3475, KEYTRUDA), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab.

102. The method of embodiment 99, wherein the anti-PD-L1 antibody is selected from the group consisting of atezolizumab (MPDL3280A, Tecentriq), avelumab (Bavencio), durvalumab (MEDI4736, Imfinzi), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

103. The method of embodiment 99, wherein the anti-CTLA-4 antibody is selected from the group consisting of ipilimumab (Yervoy), tremelimumab, AGEN1181, AGEN1884, ADU-1064, BCD-145, and BCD-217.

104. The method of embodiment 90, wherein the conjugate is administered to the subject before, concurrently with, or after the administration of the immune checkpoint inhibitor.

105. The method of any of embodiments 88-104, wherein the immune checkpoint inhibitor is administered once, twice, three times, four times, five times, six times, seven times, eight times, nine times, 10 times, or more than 10 times to the subject.

106. The method of any of embodiments 88-105, wherein the immune checkpoint inhibitor is administered once, twice, three times, four times, five times, six times, seven times, eight times, nine times, or 10 times before the administration of the conjugate, and subsequently followed by the administration of the immune checkpoint inhibitor zero time, once, twice, three times, four times, five times, six times, seven times, eight times, nine times, 10 times, or more than 10 times to the subject after administration of the conjugate.

107. The method of any of embodiments 88-105, wherein the immune checkpoint inhibitor is administered concurrently with the administration of the conjugate, and subsequently followed by the administration of the immune checkpoint inhibitor zero time, once, twice, twice, three times, four times, five times, six times, seven times, eight times, nine times, 10 times, or more than 10 times to the subject after administration of the conjugate.

108. The method of any of embodiments 88-105, wherein the immune checkpoint inhibitor is administered once, twice, three times, four times, five times, six times, seven times, eight times, nine times, 10 times, or more than 10 times after the administration of the conjugate to the subject.

109. The method of any of embodiments 88-108, wherein the phthalocyanine dye is a Si-phthalocyanine dye.

110. The method of embodiment 109, wherein the Si-phthalocyanine dye is IR700.

111. The method of any of embodiments 88-110, wherein the illumination is carried out between 30 minutes and 96 hours after administering the conjugate.

112. The method of any of embodiments 88-111, wherein the illumination is carried out 24 hours±4 hours after administering the conjugate.

113. The method of any of embodiments 88-112, wherein the primary tumor is illuminated at a wavelength of 690±40 nm.

114. The method of any of embodiments 88-113, wherein the primary tumor is illuminated at a dose of or about of 50 J/cm² or 100 J/cm of fiber length.

115. The method of any of embodiments 88-114, wherein one or more of steps (1), (2), and (3) are repeated.

116. The method of any of embodiments 88-115, further comprising (4) administering an additional therapeutic agent or anti-cancer treatment.

117. The method of any of embodiments 88-116, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

118. The method of any of embodiments 89-117, wherein the metastatic tumor cells are located in one, two, three, or more than three tissues or organs that are different from the tissue or organ where the primary tumor is located.

119. A method for treating a cancer, comprising:

(a) administering an immune checkpoint inhibitor to a subject having a cancer comprising a first tumor and a secondary population of tumor cells;

(b) administering a conjugate comprising a phthalocyanine dye linked to an anti-CD25 antibody to the subject; and

(c) after administering the conjugate, illuminating the first tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the secondary population is not directly illuminated.

120. The method of embodiment 119, wherein the first tumor and/or the secondary population is inhibited to a greater degree as compared with administration of only the conjugate, only the conjugate followed by illumination, or only the immune checkpoint inhibitor.

121. The method of embodiment 119 or 120, wherein the immune checkpoint inhibitor is selected from the group consisting of a PD-1 inhibitor, a PD-L1 inhibitor or a CTLA-4 inhibitor.

122. The method of any of embodiments 119-121, wherein the immune checkpoint inhibitor is or comprises an anti-PD-1 antibody, an anti-PD-L1 antibody or an anti-CTLA-4 antibody, or an antigen-binding fragment thereof.

123. The method of embodiment 122, wherein the immune checkpoint inhibitor is or comprises an anti-PD-1 antibody or an antigen-binding fragment thereof.

124. A method for treating a cancer, comprising:

(a) administering an anti-PD-1 antibody to a subject having a cancer comprising a first tumor and a secondary population of tumor cells;

(b) administering a conjugate comprising a phthalocyanine dye linked to an anti-CD25 antibody to the subject; and

(c) after administering the conjugate, illuminating the first tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the secondary population is not directly illuminated;

wherein the first tumor and/or the secondary population is inhibited to a greater degree as compared with administration of only the conjugate, only the conjugate followed by illumination, or only the anti-PD-1 antibody.

125. The method of any of embodiments 119-124, wherein inhibition comprises one or more of: less than at or about 20% increase in tumor volume, tumor dimensions or tumor mass, or a reduction in tumor volume, tumor dimensions or tumor mass, or a reduction in the number of tumor cells.

126. The method of embodiment 125, wherein the reduction in tumor volume, tumor dimensions or tumor mass, or the number of tumor cells comprises at or about a 30% reduction or more.

127. The method of any of embodiments 119-126, wherein the secondary population comprises metastatic tumor cells.

128. The method of any of embodiments 119-126, wherein the secondary population comprises invasive tumor cells.

129. The method of any of embodiments 119-128, wherein the secondary population comprises metastatic tumor cells and invasive tumor cells.

130. The method of any of embodiments 119-129, wherein the immune checkpoint inhibitor is administered to the subject concurrently with the conjugate.

131. The method of any of embodiments 119-130, wherein the immune checkpoint inhibitor is administered to the subject within 24 to 48 hours of administering the conjugate.

132. The method of any of embodiments 119-131, wherein the immune checkpoint inhibitor is administered to the subject within 24 hours±4 hours of administering the conjugate.

133. The method of any of embodiments 119-132, wherein a first dose of the immune checkpoint inhibitor is administered prior to the administration of the conjugate.

134. The method of embodiment 133, wherein the first dose of the immune checkpoint inhibitor is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

135. The method of any of embodiments 119-134, wherein the immune checkpoint inhibitor is administered once, twice, three times, four times, five times, six times, seven times, eight times, nine times, 10 times, or more than 10 times to the subject.

136. The method of any of embodiments 119-135, wherein immune checkpoint inhibitor is administered once, twice, three times, four times, five times, six times, seven times, eight times, nine times, or 10 times before the administration of the conjugate, and subsequently followed by the administration of the immune checkpoint inhibitor zero times, once, twice, three times, four times, five times, six times, seven times, eight times, nine times, 10 times, or more than 10 times to the subject after administration of the conjugate.

137. The method of any of embodiments 119-135, wherein the immune checkpoint inhibitor is administered concurrently with the administration of the conjugate, and subsequently followed by the administration of the immune checkpoint inhibitor zero times, once, twice, twice, three times, four times, five times, six times, seven times, eight times, nine times, 10 times, or more than 10 times to the subject after administration of the conjugate.

138. The method of any of embodiments 119-135, wherein the immune checkpoint inhibitor is administered once, twice, three times, four times, five times, six times, seven times, eight times, nine times, 10 times, or more than 10 times after the administration of the conjugate to the subject.

139. The method of any of embodiments 122-129, wherein the anti-PD-1 antibody is administered to the subject concurrently with the conjugate.

140. The method of any of embodiments 122-130, wherein the anti-PD-1 antibody is administered to the subject within 24 to 48 hours of administering the conjugate.

141. The method of any of embodiments 122-131, wherein the anti-PD-1 antibody is administered to the subject within 24 hours±4 hours of administering the conjugate.

142. The method of any of embodiments 122-132, wherein a first dose of the anti-PD-1 antibody is administered prior to the administration of the conjugate.

143. The method of embodiment 142, wherein the first dose of the anti-PD-1 antibody is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

144. The method of any of embodiments 122-143, wherein the anti-PD-1 antibody is administered once, twice, three times, four times, five times, six times, seven times, eight times, nine times, 10 times, or more than 10 times to the subject.

145. The method of any of embodiments 122-144, wherein anti-PD-1 antibody is administered once, twice, three times, four times, five times, six times, seven times, eight times, nine times, or 10 times before the administration of the conjugate, and subsequently followed by the administration of the anti-PD-1 antibody zero times, once, twice, three times, four times, five times, six times, seven times, eight times, nine times, 10 times, or more than 10 times to the subject after administration of the conjugate.

146. The method of any of embodiments 122-144, wherein the anti-PD-1 antibody is administered concurrently with the administration of the conjugate, and subsequently followed by the administration of the anti-PD-1 antibody zero times, once, twice, twice, three times, four times, five times, six times, seven times, eight times, nine times, 10 times, or more than 10 times to the subject after administration of the conjugate.

147. The method of any of embodiments 122-144, wherein the anti-PD-1 antibody is administered once, twice, three times, four times, five times, six times, seven times, eight times, nine times, 10 times, or more than 10 times after the administration of the conjugate to the subject.

148. The method of any of embodiments 119-147, wherein the secondary population is comprised in a solid tumor.

149. The method of any of embodiments 119-148, wherein the subject exhibits a complete response.

150. The method of any of embodiments 119-149, wherein the anti-CD25 antibody comprises a functional Fc region.

151. The method of any of embodiments 119-150, wherein the anti-CD25 antibody is basiliximab or daclizumab, or biosimilar, interchangeable, biobetter, copy biological or biogeneric thereof.

152. The method of embodiment 151, wherein the anti-CD25 antibody is basiliximab.

153. The method of any of embodiments 122-152, wherein the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

154. The method of any of embodiments 119-153, wherein the phthalocyanine dye is a Si-phthalocyanine dye.

155. The method of embodiment 154, wherein the Si-phthalocyanine dye is IR700.

156. The method of any of embodiments 119-155, wherein the illumination is carried out between 30 minutes and 96 hours after administering the conjugate.

157. The method of embodiment 156, wherein the illumination is carried out 24 hours 4 hours after administering the conjugate.

158. The method of any of embodiments 119-157, wherein the first tumor is illuminated at a wavelength of 690±40 nm.

159. The method of any of embodiments 119-158, wherein the first tumor is illuminated at a dose of or about of 50 J/cm² or 100 J/cm of fiber length.

160. The method of any of embodiments 119-159, further comprising (d) administering an additional therapeutic agent or anti-cancer treatment.

161. The method of any of embodiments 119-160, wherein one or more of steps (a), (b), (c), or (d) are repeated.

162. The method of any of embodiments 119-161, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

163. The method of any of embodiments 119-162, wherein the second population is located in one, two, three, or more than three tissues or organs that are different from the tissue or organ where the first tumor is located.

164. The method of any of embodiments 119-163, wherein the first tumor and/or the secondary population is inhibited to a greater degree as compared with administration of only the conjugate followed by illumination, and as compared with administration of only the immune checkpoint inhibitor.

165. The method of any of embodiments 122-164, wherein the first tumor and/or the secondary population is inhibited to a greater degree as compared with administration of only the conjugate followed by illumination, and as compared with administration of only the anti-PD-1 antibody.

166. A method for generating an enhanced response in a subject having a cancer, comprising:

(a) administering an immune checkpoint inhibitor to the subject having a cancer comprising a tumor;

(b) administering a conjugate comprising a phthalocyanine dye linked to an anti-CD25 antibody to the subject, wherein the immune checkpoint inhibitor is administered prior to or concurrently with the conjugate; and

(c) after administering the conjugate, illuminating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length.

167. The method of embodiment 166, wherein:

the enhanced response comprises an enhancement of systemic immunity of the subject as compared to the systemic immunity of the subject prior to the administration of the conjugate followed by illumination and the immune checkpoint inhibitor; and/or

the enhanced response comprises an enhancement of inhibition of the tumor as compared with administration of only the conjugate, only the conjugate followed by illumination, or only the immune checkpoint inhibitor.

168. The method of embodiment 166 or 167, wherein the immune checkpoint inhibitor is selected from the group consisting of a PD-1 inhibitor, a PD-L1 inhibitor or a CTLA-4 inhibitor.

169. The method of any of embodiments 166-168, wherein the immune checkpoint inhibitor is or comprises an anti-PD-1 antibody, an anti-PD-L1 antibody or an anti-CTLA-4 antibody, or an antigen-binding fragment thereof.

170. The method of embodiment 169, wherein the immune checkpoint inhibitor is or comprises an anti-PD-1 antibody or an antigen-binding fragment thereof.

171. A method for generating an enhanced response in a subject having a cancer, comprising:

(a) administering an anti-PD-1 antibody to the subject having a cancer comprising a tumor;

(b) administering a conjugate comprising a phthalocyanine dye linked to an anti-CD25 antibody to the subject, wherein the anti-PD-1 antibody is administered prior to or concurrently with the conjugate; and

(c) after administering the conjugate, illuminating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length;

wherein:

the enhanced response comprises an enhancement of systemic immunity of the subject as compared to the systemic immunity of the subject prior to the administration of the conjugate followed by illumination and the anti-PD-1 antibody; and/or

the enhanced response comprises an enhancement of inhibition of the tumor as compared with administration of only the conjugate, only the conjugate followed by illumination, or only the anti-PD-1 antibody.

172. The method of any of embodiments 166-171, wherein the enhanced response is additive or synergistic.

173. The method of any of embodiments 166-172, wherein inhibition comprises one or more of: less than at or about 20% increase in tumor volume, tumor dimensions or tumor mass, or a reduction in tumor volume, tumor dimensions or tumor mass, or a reduction in the number of tumor cells.

174. The method of embodiment 173, wherein the reduction in tumor volume, tumor dimensions or tumor mass, or the number of tumor cells comprises at or about a 30% reduction or more.

175. The method of any of embodiments 166-174, wherein:

the tumor comprises a first tumor and a secondary population of tumor cells, and wherein the first tumor is illuminated and the secondary population is not directly illuminated;

the tumor comprises a first tumor and metastatic tumor cells, and wherein the first tumor is illuminated and the metastatic tumor cells are not directly illuminated; and/or

the tumor comprises a first tumor and invasive tumor cells, and wherein the first tumor is illuminated and the invasive tumor cells are not directly illuminated.

176. The method of any of embodiments 167-175, wherein the enhanced response is a synergistic response, wherein the synergistic response comprises a synergistic reduction in the growth, tumor volume, tumor dimensions or tumor mass of a first tumor, a synergistic reduction in the number of cells in the secondary population in the subject, a synergistic reduction in the growth, tumor volume, tumor dimensions, tumor mass, or the number of metastatic or invasive tumor cells, or any combination thereof.

177. The method of any of embodiments 166-176, wherein the immune checkpoint inhibitor is administered to the subject within 24 hour to 48 hours of administering the conjugate.

178. The method of any of embodiments 166-177, wherein the immune checkpoint inhibitor is administered to the subject within 24 hours±4 hours of administering the conjugate.

179. The method of any of embodiments 166-178, wherein a first dose of the immune checkpoint inhibitor is administered prior to the administration of the conjugate.

180. The method of embodiment 179, wherein the first dose of the immune checkpoint inhibitor is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

181. The method of any of embodiments 169-180, wherein the anti-PD-1 antibody is administered to the subject within 24 hour to 48 hours of administering the conjugate.

182. The method of any of embodiments 169-181, wherein the anti-PD-1 antibody is administered to the subject within 24 hours±4 hours of administering the conjugate.

183. The method of any of embodiments 169-182, wherein a first dose of the anti-PD-1 antibody is administered prior to the administration of the conjugate.

184. The method of embodiment 183, wherein the first dose of the anti-PD-1 antibody is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

185. The method of any of embodiments 166-184, wherein systemic immunity is measured by one or more of a cytotoxic T lymphocyte (CTL) activity assay, an intratumoral T cell exhaustion assay, an intratumoral effector T cell expansion assay, a T cell receptor diversity assay, an activated CD8⁺ T cell assay, a circulating regulatory T cell (Treg) assay, an intratumoral Treg assay, or a CD8⁺ Tcell:Treg assay.

186. The method of any of embodiments 166-185, wherein systemic immunity is measured by a CTL activity assay using splenocytes or peripheral blood cells or bone marrow cells or lymph node cells, optionally collected from the subject between day 4 and day 28 after illumination of the first tumor in the subject.

187. The method of any of embodiments 166-185, wherein systemic immunity is measured by an intratumoral T cell exhaustion assay using T cells collected from the first tumor or a metastatic tumor cell mass or an invasive tumor cell mass, optionally collected from the subject between day 4 and day 28 after illumination of the first tumor in the subject.

188. The method of any of embodiments 166-185, wherein systemic immunity is measured by an intratumoral effector T cell expansion assay using T cells collected from the first tumor or a metastatic tumor cell mass or an invasive tumor cell mass, optionally collected from the subject between day 4 and day 28 after illumination of the first tumor in the subject.

189. The method of any of embodiments 166-185, wherein systemic immunity is measured by a T cell receptor diversity assay using T cells collected from the first tumor or a metastatic tumor cell mass or an invasive tumor cell mass or the peripheral circulation, optionally collected from the subject between day 4 and day 28 after illumination of the first tumor in the subject.

190. The method of any of embodiments 166-185, wherein systemic immunity is measured by determining the presence, number or frequency of regulatory T cells (Tregs) in the tumor and/or the ratio of intratumoral Treg cells to intratumoral CD8+ T cells or intratumoral CD4+ T cells from the first tumor or a metastatic tumor cell mass or an invasive tumor cell mass, optionally collected from the subject between day 4 and day 28 after illumination of the first tumor in the subject.

191. The method of any of embodiments 166-190, wherein the anti-CD25 antibody comprises a functional Fc region.

192. The method of any of embodiments 166-191, wherein the anti-CD25 antibody is basiliximab or daclizumab, or biosimilar, interchangeable, biobetter, copy biological or biogeneric thereof.

193. The method of embodiment 192, wherein the anti-CD25 antibody is basiliximab.

194. The method of any of embodiments 169-193, wherein the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

195. The method of any of embodiments 166-194, wherein the immune checkpoint inhibitor is administered once, twice, three times, four times, five times, six times, seven times, eight times, nine times, 10 times, or more than 10 times to the subject.

196. The method of any of embodiments 169-195, wherein the anti-PD-1 antibody is administered once, twice, three times, four times, five times, six times, seven times, eight times, nine times, 10 times, or more than 10 times to the subject.

197. The method of any of embodiments 166-196, wherein the phthalocyanine dye is a Si-phthalocyanine dye.

198. The method of embodiment 197, wherein the Si-phthalocyanine dye is IR700.

199. The method of any of embodiments 166-198, wherein the illumination is carried out between 30 minutes and 96 hours after administering the conjugate.

200. The method of embodiment 199, wherein the illumination is carried out 24 hours±4 hours after administering the conjugate.

201. The method of any of embodiments 166-200, wherein the tumor is illuminated at a wavelength of 690±40 nm.

202. The method of any of embodiments 166-201, wherein the tumor is illuminated at a dose of or about of 50 J/cm² or 100 J/cm of fiber length.

203. The method of any of embodiments 166-202, further comprising (d) administering an additional therapeutic agent or anti-cancer treatment.

204. The method of any of embodiments 166-203, wherein one or more of steps (a), (b), (c), or (d) are repeated.

205. The method of any of embodiments 166-204, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

206. The method of any of embodiments 166-205, wherein the enhanced response comprises an additive response or a synergistic response as compared with administration of only the conjugate followed by illumination, and as compared with administration of only the immune checkpoint inhibitor.

207. The method of any of embodiments 166-206, wherein the enhanced response comprises an additive response or a synergistic response as compared with administration of only the conjugate followed by illumination, and as compared with administration of only the anti-PD-1 antibody.

### IX. Examples

The following examples are included for illustrative purposes only and are not intended to limit the scope of the invention.

### Example 1 Generation of Anti-CD25 Antibody-IRDye 700 Conjugate

This example describes a method for preparing a conjugate containing IRDye 700DX (IR700) linked to an exemplary anti-CD25 antibody, PC61, thus producing a PC61-IRDye 700DX (PC61-IR700 conjugate).

PC61, a rat monoclonal antibody (mAb) directed against mouse CD25, was incubated (1 mg, 6.8 nmol) with IRDye 700DX NHS Ester (IR700; LI-COR Bioscience, Lincoln, Nebr.) (66.8 μg, 34.2 nmol, 5 mmol/L in DMSO) in 0.1 mol/L Na₂HPO₄ (pH 8.5) at room temperature for 30 to 120 min. The mixture was purified using a Sephadex G50 column (PD-10; GE Healthcare, Piscataway, N.J.). Protein concentration was determined with Coomassie Plus protein assay kit (Pierce Biotechnology, Rockford, Ill.) by measuring the absorption at 595 nm with a UV-Vis system (8453 Value System; Agilent Technologies, Palo Alto, Calif.). The concentration of IR700 was measured by absorption with the UV-Vis system to confirm the number of fluorophore molecules conjugated to each PC61 molecule. The number of IR700 per PC61 was about 3.

Purity of the PC61-IR700 conjugate was confirmed by analytical size-exclusion HPLC (SE-HPLC). SE-HPLC was performed using an Agilent 1100 HPLC system (Santa Clara, Calif.) equipped with a PDA detector controlled by Chemstation software. SE chromatography was performed on a Shodex KW-803 column (New York, N.Y.) eluted for 20 minutes using phosphate buffered saline (PBS) at 1.0 mL/min. The PC61-IR700 preparation exhibited strong association and contained no detectable mAb aggregates as determined by SE-HPLC.

To determine the in vitro binding characteristics of IR700 conjugates, ¹²⁵I labeling of the conjugates using the Indo-Gen procedure was performed. Minimal loss of mAb with IR700 conjugation was observed. Immunoreactivity assay was performed as described previously. Briefly, after trypsinization, 2×10⁶ of tumor cells were resuspended in PBS containing 1% bovine serum albumin (BSA). ¹²⁵I-PC61-IR700 (1 mCi, 0.2 μg) was added and incubated for 1 h on ice. The cells were washed, pelleted, the supernatant was decanted, and the cells were counted in a 2470 Wizard gamma-counter (Perkin Elmer, Shelton, Conn.). Nonspecific binding to the cells was examined under conditions of excess unlabeled antibody (200 μg of non-labeled PC61).

### Example 2 Anti-CD25-IR700 PIT and Anti-PD-1 Antibody Synergistically Inhibit Tumor Growth and Survival In Vivo

This example describes the synergistic inhibitory effect of an exemplary anti-CD25 antibody-IR700 PIT in combination with an exemplary anti-PD-1 antibody on tumor growth in vivo.

Immunocompetent BALB/c mice at age of 6-8 weeks were inoculated with 3×10⁶ CT26-EphA2 clone c4D10 murine colon carcinoma cells/mouse subcutaneously on the right hind flank. When allograft tumors grew to about 150 mm³, the mice were administered with saline (100 μL), PC61-IR700 conjugate (100 μg) generated substantially as described in Example 1 above, anti-PD-1 antibody clone RMP1-14 (100 μg), or a combination of PC61-IR700 conjugate (100 μg) and RMP1-14 (100 μg). The PC61-IR700 conjugate was administered at day 4 and RMP1-14 at days 4, 6, 8, and 11. Twenty-four hours after administration of PC61-IR700 conjugate, tumors in the PIT group were illuminated at 690 nm at a dosage of 100 J/cm². The tumor growth and survival were observed over 22 days. Tumor volume was calculated using the formula: tumor volume=(width×length)×height/2.

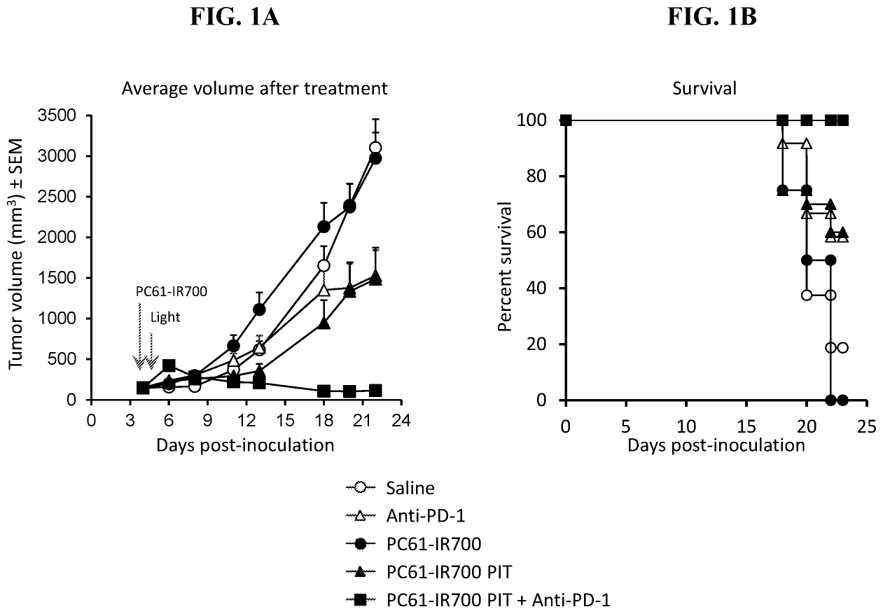

In mice that received the PC61-IR700-PIT or anti-PD-1 (RMP1-14) monotherapies, the growth of tumors was substantially inhibited in comparison to the tumor growth in control mice that received saline or PC61-IR700 conjugate alone without PIT (FIG. 1A; closed and open triangles compared to open and circles, respectively). Strikingly, in mice treated with a combination of PC61-IR700-PIT and anti-PD-1 antibody, tumor growth was further inhibited in a synergistic manner (FIG. 1A, closed squares). In addition to examining the overall tumor growth, the rate of complete response (CR) was also compared among the treatment groups. After 25 days of treatment, 7/20 (35%) of mice treated with PC61-IR700 PIT and 1/12 (8.3%) of mice treated with anti-PD-1 (RMP1-14) monotherapy achieved CR, while 1/16 (6.25%) or 0/12 (0%) of the animals in the saline only or PC61-IR700 conjugate alone without PIT control groups achieved CR. Surprisingly, the combination of PC61-IR700 conjugate and anti-PD-1 treatment resulted in CR in (17/23) 73.9% of mice (FIG. 1A). The combination therapy had substantially greater rate of CR than either PC61-IR700-PIT or anti-PD-1 monotherapies, and the effect of these two treatments was synergistic. Corroborating these results, mice receiving PC61-IR700-PIT or anti PD-1 (RMP1-14) monotherapies (closed triangle and closed circle, respectively), exhibited increased survival compared to control mice that received saline or PC61-IR700 conjugate alone without PIT (FIG. 1B, closed and open triangles compared to open and circles, respectively), and mice treated with a combination of PC61-IR700 PIT and PD-1 antibody exhibited even better (100%) survival (FIG. 1B, closed squares). These results support synergistic effects between anti-CD25 PIT and anti-PD-1 therapy.

### Example 3 Anti-CD25-IR700 PIT and Anti-PD-1 Synergistically Inhibit the Growth of Non-illuminated Distal Tumors In Vivo

This example describes the synergistic inhibitory effect of an exemplary anti-CD25 antibody-IR700 PIT in combination with an exemplary anti-PD-1 antibody, RMP1-14, on the growth of distal tumors that were not illuminated.

BALB/c mice at age of 6-8 weeks were inoculated with 3×10⁶ CT26-EphA2 clone c4D10 cells/mouse subcutaneously on both right and left hind flanks. When allograft tumors on both sides of the mice grew to a size of about 150 mm³, mice were administered saline (100 μL), anti-CD25 antibody PC61-IR700 conjugate (100 μg), RMP1-14 (100 μg), or a combination of PC61-IR700 conjugate (100 μg) and RMP1-14 (100 μg). The PC61-IR700 conjugate was administered at day 4 and RMP1-14 at days 4, 6, 8, and 11. Twenty-four hours after administration of PC61-IR700 conjugate, tumors on the right flanks in the mice of PC61-IR700 PIT group were illuminated at 690 nm at 100 J/cm², while those on the left flanks were not illuminated. Thus, the right flank tumors served as primary tumors, and the left flank ones served as distal or metastatic tumors.

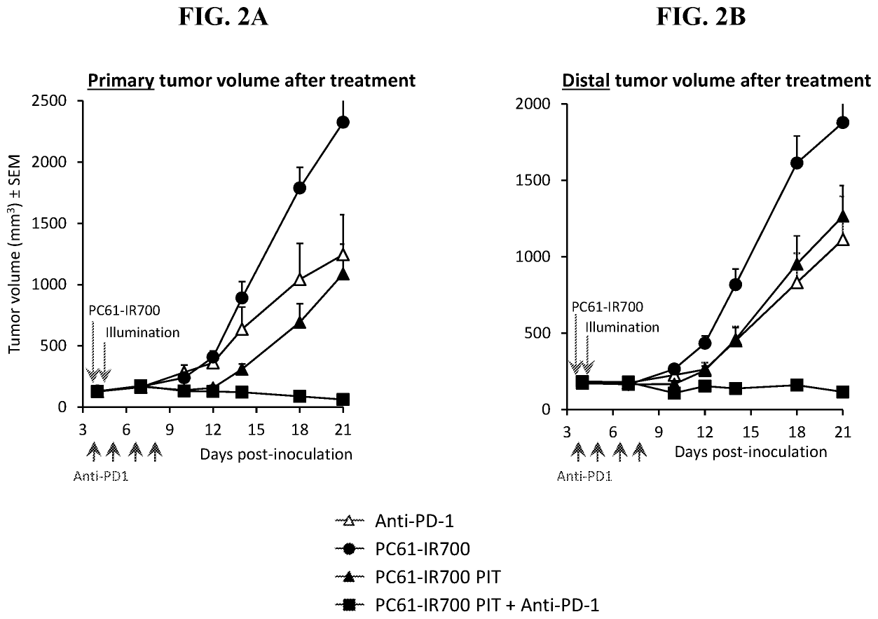

As shown in FIGS. 2A-2B, in mice treated with PC61-IR700 conjugate with illumination (PC61-IR700 PIT; closed triangles) or RMP1-14 monotherapy (open triangles), the growth of the illuminated primary tumors (FIG. 2A) and the distal, non-illuminated tumors (FIG. 2B) was substantially inhibited when compared to those treated PC61-IR700 conjugate without illumination (closed circles). In mice treated with the combination of PC61-IR700 conjugate with illumination (PC61-IR700 PIT) and anti-PD-1 (RMP1-14), the growth of the illuminated primary tumors (FIG. 2A) and the distal, non-illuminated tumors (FIG. 2B) was further inhibited, and this inhibitory effect was synergistic (closed squares).

### Example 4 Anti-CD25 Antibody Effects on Circulating Regulatory T Cell Levels

This example describes the effects of an exemplary anti-CD25 antibody on circulating regulatory T cells (Tregs).

BALB/c mice were administered 100 μg anti-CD25 antibody PC61 antibody (PC61 WT; n=8), or PC61 antibody with the rat Fc region replaced with a wild-type mouse Fc region (PC61 mouse WT; n=8) or an N297Q mutant mouse Fc region that lacks effector function/ADCC activity (PC61 mouse N297Q; n=8). Control mice were administered 100 μl saline for comparison (n=6). Blood samples were collected on day 1 and 8 after antibody administration, and isolated lymphocytes were stained for cell markers including CD25, FoxP3, CD3, and CD4. Isotype controls were also used for staining. The stained cells were analyzed using flow cytometry, and the percentage of CD25⁺FoxP3⁺ of CD3⁺CD4⁺ T cells was determined for each condition.

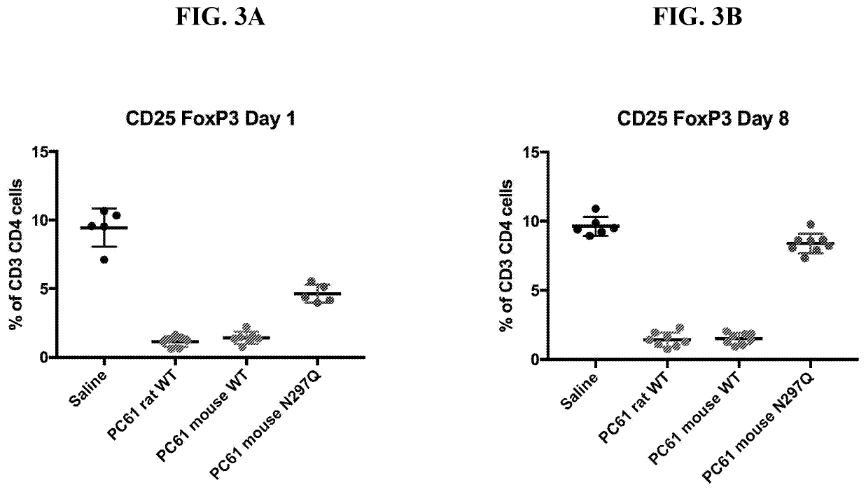

The percentage of CD25⁺FoxP3⁺ T cells in mice administered PC61 with a wild-type rat or mouse Fc domain was reduced compared to the saline controls the day after administration (FIG. 3A), and the reduction was sustained at day 8 (FIG. 3B). The percentage of CD25⁺FoxP3⁺ T cells was reduced to a lesser extent the day after administration of PC61 with a mutant Fc (PC61 mouse N297Q; FIG. 3A) than the antibodies with the wild-type Fc domains. Unlike the mice treated with the antibodies with the wild-type Fc domains, the reduction following PC61 mouse N297Q administration was transient, as the percentage of CD25⁺FoxP3⁺ T cells recovered similar to control levels by day 8 (FIG. 3B).

### Example 5 Anti-CD25-IR700 PIT and Anti-PD-1 Synergistic Effects on Non-Illuminated Distal Tumors

This example describes the role of circulating regulatory T cells (T_{reg}) depletion in the synergistic anticancer effects of an exemplary anti-CD25 antibody-IR700 PIT in combination with an exemplary anti-PD-1 antibody (RMP1-14) on the growth of distal tumors that were not illuminated.

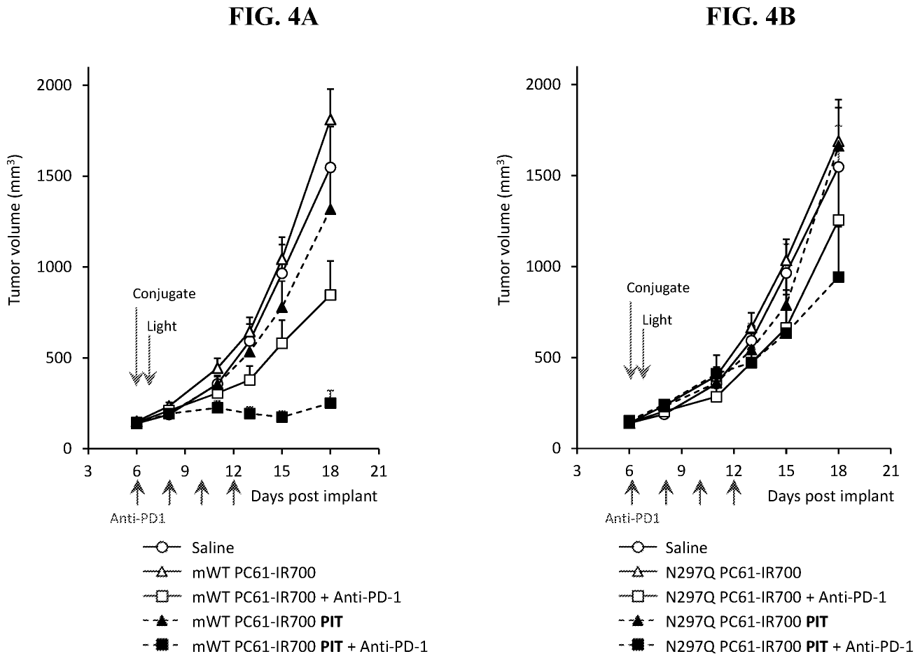

BALB/c mice at age of 6-8 weeks were inoculated with 3×10⁶ CT26-EphA2 clone c4D10 cells/mouse subcutaneously on both right and left hind flanks. When allograft tumors on both sides of the mice grew to a size of about 130 mm³-140 mm³ (Day 6 post implant), mice were administered saline (100 μL), anti-CD25 antibody PC61-IR700 conjugate containing a mouse wild-type (mWT) Fc region (mWT PC61-IR700) or N297Q mutant Fc region (N297Q), lacking effector function, (N297Q PC61-IR700) (100 μg), anti-PD-1 (RMP1-14; 100 μg), a combination of PC61-IR700 conjugate containing a WT or N297Q Fc region (100 μg) and anti-PD-1 (100 μg). The mWT PC61-IR700 and N297Q conjugates were administered at Day 6 and RMP1-14 at Day 6, 8, 10 and 12. Twenty-four hours after administration of PC61-IR700 conjugate, tumors on the right flanks in the mice of PC61-IR700 PIT group were illuminated at 690 nm at 100 J/cm², while those on the left flanks were shielded with black foil to prevent illumination. Thus, the right flank tumors served as primary tumors, and the left flank tumors served as unilluminated, distal tumors. Tumor volumes were measured every 2-3 days until 18 days post implant. The results for mice administered treatments involving the conjugate with an mWT Fc domain are shown in FIG. 4A. Results for mice administered treatments involving the conjugate with an ADCC-incompetent mutant N297Q Fc domain are shown in FIG. 4B.

Mice administered saline only (open circles; FIGS. 4A and 4B) exhibited progressive tumor growth over the course of the study. Mice administered conjugate only (mWT or N297Q) (open triangles, FIGS. 4A and 4B, respectively) also exhibited progressive tumor growth. Combinatorial administration of conjugate (mWT or N297Q) and anti-PD-1, without illumination (open squares, FIGS. 4A and 4B, respectively), resulted in some reduced tumor growth compared to saline controls. PIT with the mWT conjugate (closed triangles, FIG. 4A) resulted in reduced tumor growth compared to saline controls, while PIT with the ADCC-incompetent N297Q conjugate (closed triangles, FIG. 4B) had no effect on tumor growth. The combination of PIT with the mWT conjugate and anti-PD-1 treatment (closed squares, FIG. 4A) substantially arrested tumor growth. The efficacy of the PIT-anti-PD-1 combination treatment was substantially diminished when administering the N297Q conjugate (closed squares, FIG. 4B). These results, taken together with those from Example 4, indicate that the anticancer activity of the anti-CD25-IR700 PIT as a monotherapy or in combination with anti-PD-1 treatment is significantly enhanced with concurrent, sustained depletion of circulating T_{regs}.

### Example 6 Anti-CD25-IR700 PIT Effect on Intratumoral Regulatory T Cells

This example describes the depletion of regulatory T cells (T_{regs}) in vivo in response to an exemplary anti-CD25 antibody-IR700 PIT.

BALB/c mice were inoculated with 1×10⁶ 4T1-EpCAM tumor cells subcutaneously on the right hind flank. Once tumors reached an average volume of approximately 180 mm³, mice were treated with saline, anti-CD25 antibody PC61-IR700 conjugate alone or PC61-IR700 conjugate with illumination (PC61-IR700 PIT). Twenty-four hours after administration of the conjugate, tumors in the mice of the illuminated (PIT) group were exposed to 690 nm light at 100 J/cm². Two hours post illumination, tumors were excised from all groups and processed into single cell suspensions. Suspended cells were then stained the T_{reg} markers CD3, CD4, CD45, and FoxP3. The stained cells were analyzed using flow cytometry, and the percentage of FoxP3 cells out of CD3⁺CD4⁺ lymphocytes was determined.

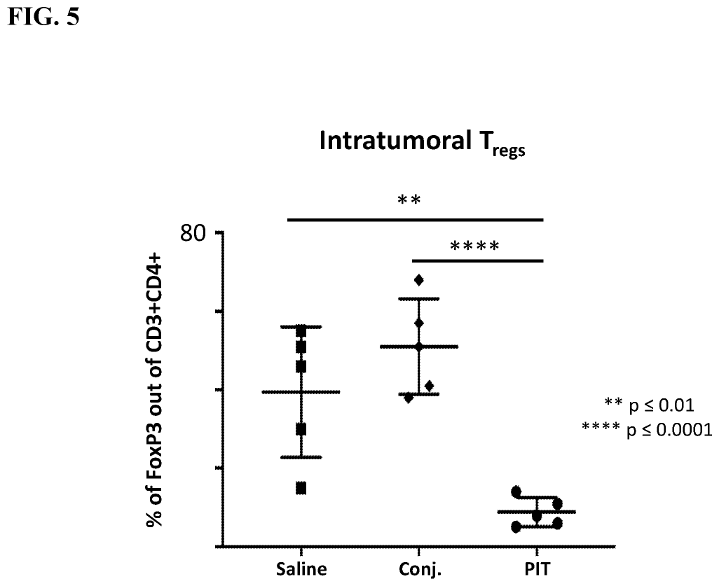

As shown in FIG. 5, in tumor-bearing mice treated with PC61-IR700 PIT (circles), the number of FoxP3⁺CD3⁺CD4⁺ T cells in the tumors decreased significantly in comparison to those treated with saline (squares) or PC61-IR700 conjugate alone (diamonds) (P<0.01 and P<0.0001, respectively), indicating an immediate Treg reduction in the tumor after anti-CD25 PIT (FIG. 5). No reduction in CD8⁺ T cells was observed (data not shown). The results showed that an anti-CD25 PIT leads to an acute depletion of intratumoral regulatory T cells (T_{regs}).

### Example 7 Anti-CD25-IR700 PIT Alleviates Intratumoral CD8⁺ T Cell Exhaustion In Vivo

This example describes the alleviative effect of an exemplary anti-CD25 antibody—IR700 PIT, on intratumoral CD8⁺ T cell exhaustion in vivo.

BALB/c mice were inoculated with CT26-EphA2 clone c4D10 tumor cells. Once tumors reached an average volume of 150 mm³, mice were treated with saline, anti-CD25 antibody PC61-IR700 conjugate alone, or PC61-IR700 conjugate with illumination (PC61-IR700 PIT). Twenty-four hours after administration of the conjugate, tumors in the mice of the illumination (PIT) group were illuminated at 690 nm at 100 J/cm². Two days post illumination, tumors were excised from all groups and processed into single cell suspensions. Suspended cells were then stained for cell markers including CD3, CD45, CD8a, PD-1, and CTLA4. Isotype controls were also used for staining. The stained cells were analyzed using flow cytometry.

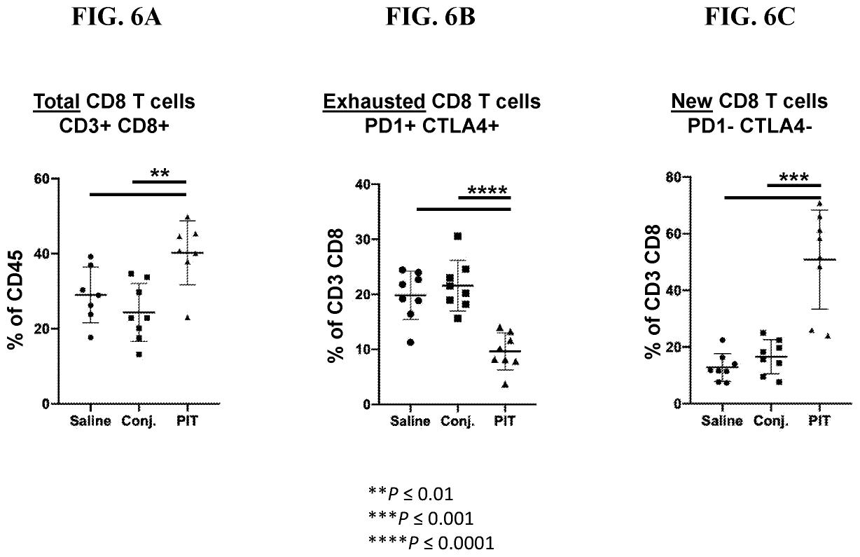

As shown in FIGS. 6A-6B, in mice treated with PC61-IR700 PIT, the proportion of intratumoral CD8⁺ T cells (CD3⁺ CD8⁺) increased significantly (P<0.01) in comparison to that in mice that received saline or PC61-IR700 conjugate alone (Conj.; without illumination) (FIG. 6A). The proportion of exhausted CD8⁺ cells (PD1⁺CTLA-4⁺) decreased significantly following PC61-IR700 PIT (P<0.0001) compared to saline or conjugate alone control (FIG. 6B).

### Example 8 Anti-CD25-IR700 PIT Increases the Non-Exhausted Intratumoral Effector CD8⁺ T Lymphocytes In Vivo

This example describes the stimulatory effect of an exemplary anti-CD25 antibody-IR700 PIT, on the expansion of intratumoral effector CD8+ T lymphocytes in vivo.

BALB/c mice were inoculated with CT26-EphA2 clone c4D10 tumor cells. Once tumors reached an approximate average volume of 150 mm³, mice were treated with saline, PC61-IR700 conjugate alone or PC61-IR700 conjugate with illumination (PC61-IR700 PIT). Twenty-four hours after administration of the conjugate, tumors in the mice of the illuminated (PIT) group were exposed to 690 nm light at 100 J/cm². Eight days post illumination, tumors were excised from all groups and processed into single cell suspensions. Suspended cells were then stained for cell markers including CD3, CD45, CD8a, PD-1, and CTLA4. Isotype controls were also used for staining. The stained cells were analyzed using flow cytometry.

As shown in FIG. 6C, in tumor-bearing mice treated with PC61-IR700 PIT, the number of non-exhausted PD-1⁻ CTLA-4⁻ CD3⁺CD8⁺ T cells in the tumors increased significantly in comparison to those administered only saline or PC61-IR700 conjugate alone (Conj.; without illumination) (P<0.001), indicating PC61-IR700 PIT induced expansion of effector CD8⁺ T cells in the tumor microenvironment (FIG. 6C).

### Example 9 Anti-CD25-IR700 PIT Results in Increased Activated CD8⁺ T Cells

This example describes the effect of an exemplary anti-CD25 antibody-IR700 PIT on intratumoral CD8⁺ T cell activation in vivo.

BALB/c mice were inoculated with 4T1-EpCAM tumor cells. Once tumors reached an approximate average volume of 150 mm³, mice were treated with saline, anti-CD25 antibody PC61-IR700 conjugate alone, or PC61-IR700 conjugate with illumination (PC61-IR700 PIT). Twenty-four hours after administration of the conjugate, tumors in the mice of the illumination (PIT) group were illuminated at 690 nm at 100 J/cm². Seven days post illumination, tumors were excised from all groups and processed into single cell suspensions. Suspended cells were then stained for cell markers including CD3, CD69, CD45, CD8a, Ki67. Isotype controls were also used for staining. The stained cells were analyzed using flow cytometry.

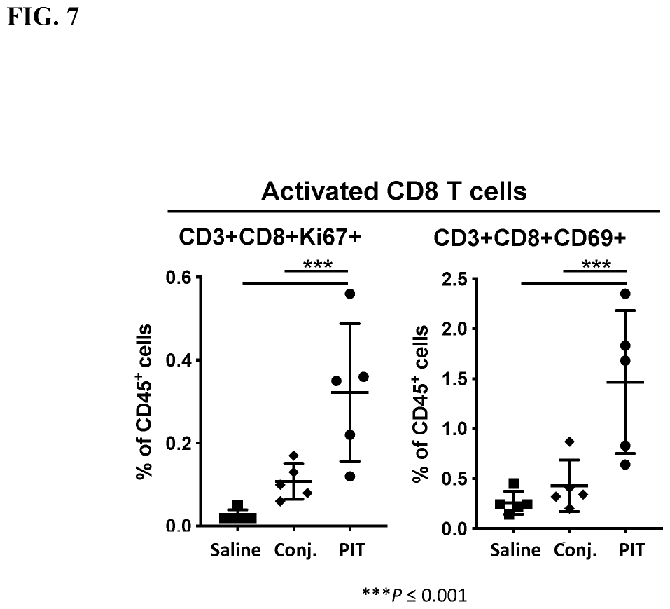

As shown in FIG. 7, in mice treated with PC61-IR700 PIT (circles), the number of activated CD8⁺ T cells (CD3⁺CD8⁺Ki67⁺, left panel; and CD3⁺CD8⁺CD69⁺, right panel) was significantly increased (P<0.001) compared to mice that received saline (squares) or PC61-IR700 conjugate alone (Conj.; without illumination) (diamonds). These results indicate that anti-CD25 PIT results in increased activated CD8 T cells in the illuminated tumor.

### Example 10 Anti-CD25-IR700 PIT Results in Sustained Intratumoral Increased CD8⁺/T_{reg} Ratio

This example describes the effect of the anti-CD25 PIT, on the ratio of intratumoral CD8⁺ T cells to regulatory T cells (T_{regs}) in vivo, which is a predictive marker of clinical response to treatment.

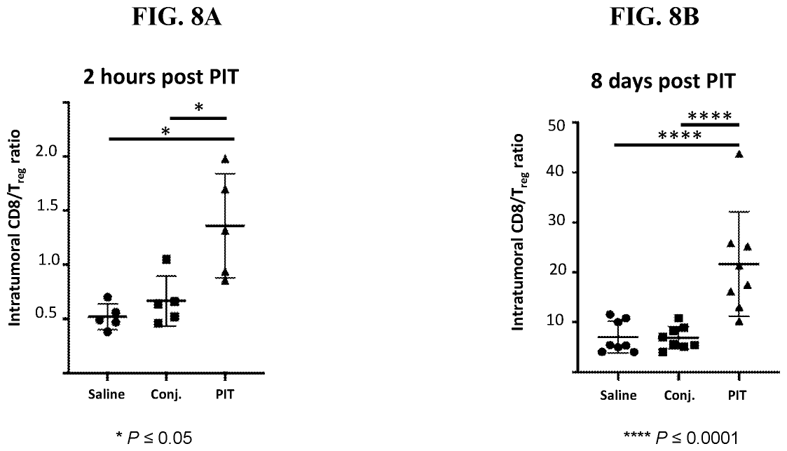

BALB/c mice were inoculated with 1×10⁶ CT26-EphA2 clone c4D10 tumor cells subcutaneously on the right hind flank. Once tumors reached an approximate average volume of 150 mm³, mice were treated with saline, PC61-IR700 conjugate (100 μg) alone (Conj.), or PC61-IR700 conjugate (100 μg) with illumination (PC61-IR700 PIT). Twenty-four hours after administration of the conjugate, tumors in the mice of the illumination (PIT) group were illuminated at 690 nm at 100 J/cm². At two hours or eight days post illumination, tumors were excised from all groups and processed into single cell suspensions. Suspended cells were then stained for cell markers including CD3, CD45, CD8a, CD4, and FoxP3. Isotype controls were also used for staining. The stained cells were analyzed using flow cytometry, and the ratio of intratumoral CD8⁺ T cells to T_{regs} was determined (FIGS. 8A-8B).

As shown in FIG. 8A, at 2 hours post illumination, tumors of mice treated with PC61-IR700 PIT (triangles) had an increased intratumoral CD8⁺/T_{reg} ratio compared to mice that received saline (circles; P≤0.05) or PC61-IR700 conjugate alone (without illumination) (squares; P≤0.05). These results indicate that anti-CD25 PIT results in intratumoral CD8 T cell activation. The increased CD8⁺/T_{reg} ratio in tumors of animal receiving PIT was sustained through eight days post illumination (FIG. 8B; P<0.0001). These results indicate a single treatment of anti-CD25 PIT results in a durable increase in CD8⁺/T_{reg} ratio inside the treated tumor.

### Example 11 Rejection of the Growth of Re-challenged Tumors in Mice with Complete Response After Anti-CD25-PIT700 PIT and Anti-PD-1 Treatments

This example describes the rejection of growth of tumors newly inoculated into the mice that had achieved complete response after initial treatment with an exemplary anti-CD25 antibody-IR700 PIT alone, or in combination with an exemplary anti-PD-1 antibody (RMP1-14) treatments.

BALB/c mice at age of 6-8 weeks were inoculated with 3×10⁶ CT26-EphA2 clone c4D10 cells/mouse subcutaneously on the right hind flank. When allograft tumors grew to a size of about 150 mm³, mice were administered with saline (100 μL), anti-CD25 antibody PC61-IR700 conjugate (100 μg), anti-PD-1 antibody RMP1-14 (100 μg), or a combination of PC61-IR700 conjugate (100 μg) and RMP1-14 (100 μg). The PC61-IR700 conjugate was administered at day 4 and RMP1-14 at days 4, 6, 8, and 11. Twenty-four hours after administration of PC61-IR700, tumors were illuminated at 690 nm at 100 J/cm². The mice that achieved complete response (with disappearance of tumors) (CR mice) and naïve mice were re-challenged with CT26-EphA2 cells on the left hind flank on day 42 after initial tumor inoculation (day 38 post treatment). For animals that exhibited no tumor growth after re-challenge with CT26-EphA2 cells, 4T1 tumors, a syngeneic mouse tumor line from a different origin than CT26, were inoculated in the right axilla on day 78 post initial tumor inoculation (day 74 post treatment).

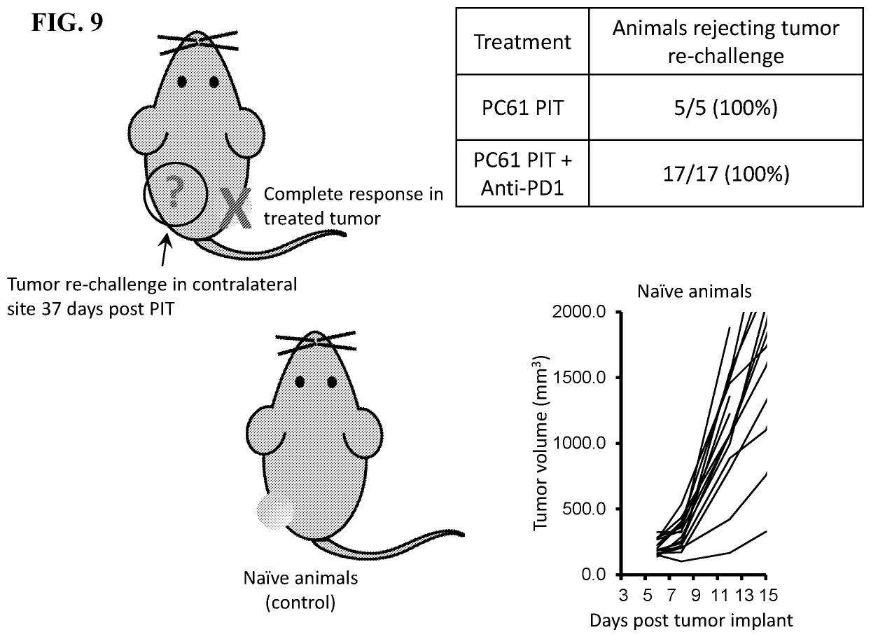

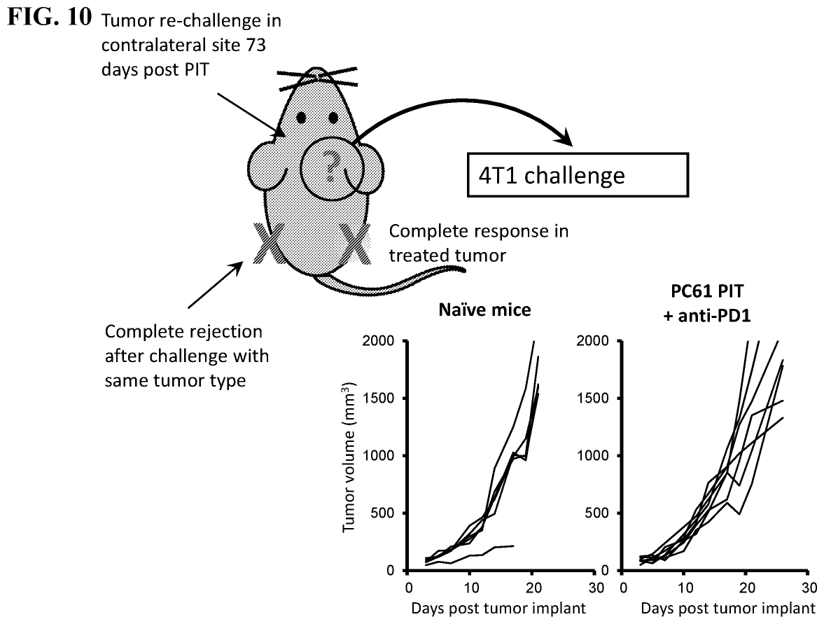

In the CR mice that were re-challenged with the same type of tumor cells CT26-EphA2, the tumor cells were completely rejected and no tumor growth was observed in groups that initially treated with PC61-IR700 PIT alone (5/5, 100%), and with the combination of PC61-IR700 PIT and RMP1-14 (17/17, 100%), while the tumor volume increased in naïve mice that were not previously exposed to treatments (FIG. 9). Following inoculation of 4T1 tumor cells in the right axilla, a different type of tumor cells from the originally inoculated, the growth of 4T1 tumors similarly increased between the treated mice and the naïve mice that were not previously exposed to treatments (FIG. 10). These data indicated that the rejection of re-challenged tumor cell growth was tumor specific.

### Example 12 Anti-CD25-IR700 PIT and Anti-PD-1 Enhance Systemic Immunity

This example describes the stimulatory effect of an exemplary anti-CD25 antibody-IR700 PIT, in combination with an exemplary anti-PD-1 antibody on systemic immunity in vivo.

A. Cytotoxic T Lymphocyte (CTL) Assay

CTL assay was designed to evaluate the tumor-specific cytotoxic activity of splenocytes from mice inoculated with CT26-EphA2 clone c4D10 tumors. Cytotoxicity was evaluated using the CytoTox™ 96 Non-Radioactive Cytotoxicity Assay (Promega; Cat. #G1780. The kit measured the levels of lactate dehydrogenase (LDH) in the well, which is released from cells upon cell death. The spleens were harvested from the tumor-bearing mice that achieved complete response (CR mice) after treatment with anti-CD25 antibody PC61-IR700 PIT monotherapy or with PC61-IR700 PIT and anti-PD-1 (RMP1-14) combination therapy, or from tumor-bearing mice that were treatment naïve. Single-cell suspensions were prepared by mechanical dissociation of the spleens over a 70-μm pore size cell strainer. The resulting flow-through was collected, and red blood cells were lysed. The suspended splenocytes were primed with the CT26 antigen AH1 peptide for four days in vitro. Afterwards, a cytotoxic assay was performed by co-incubating the splenocytes (effector cells) and CT26-ephA2 clone c4D10 target cells at a variety of effector cell to target cell ratios (E:T ratios) for four hours. Subsequently, the splenocytes were removed, and the LDH levels released from the target cells were measured. The human pancreatic cancer cell line BxPC3 cells were used as unrelated control target cells.

B. Results

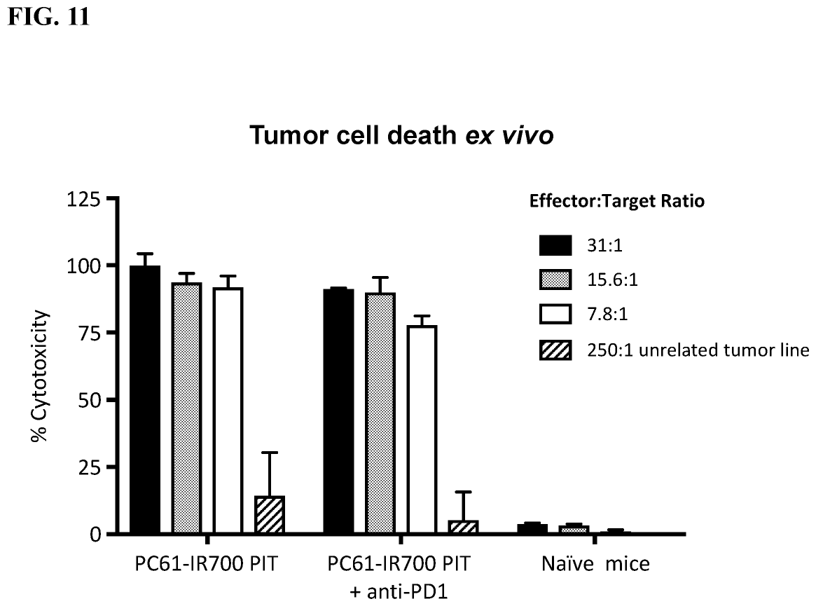

In CR mice that were treated with PC61-IR700 PIT alone or in combination with RMP1-14, the splenocytes exhibited tumor-specific immune response against the target tumor cells ex vivo (FIG. 11). For splenocytes derived from CR mice that were treated with PC61-IR700 PIT alone, the results showed a clear E:T ratio-dependent cytotoxic effect on the target tumor cells, capable of killing 100% of the target cells at an E:T ratio of 31:1, and 92% at an E:T ratio of 7.8:1 (FIG. 11). For splenocytes derived from CR mice previously treated with PC61-IR700 PIT with RMP1-14, the results showed a clear E:T ratio dependent cytotoxic effect on the target tumor cells, capable of killing 91% of the target cells at a ratio of 31:1, and 75% at a ratio of 7.8:1. For splenocytes derived from naïve mice that received no treatment, the results showed a minimal cytotoxic effect on the target tumor cells, capable of killing no more than 11% of the target cells at any E:T ratio (FIG. 11). Moreover, there was less than 15% cytotoxic effect against BxPC3 cells, an unrelated type of tumor cells serving as a control for target tumor cells, at E:T ratio as high as 250:1. These results clearly showed that treatment with anti-CD25-IR700 PIT alone or in combination with anti-PD-1 resulted in an increase in tumor-specific cytotoxic T cells in the spleen.

### Example 13 CD8⁺ T Cell Activity and the Efficacy of Anti-CD25-Conjugate and Anti-PD-1 Treatments

This example demonstrates that the effect of an exemplary anti-CD25 antibody-IR700 PIT, in combination with an exemplary anti-PD-1 antibody, on tumor growth in vivo was dependent on a functional CD8⁺ T cell population.

BALB/c mice were inoculated with 3×10⁶ CT26-EphA2 clone c4D10 cells subcutaneously on both the right and left hind flanks per mouse. Mice with CD8⁺ T cell depletion were generated by intraperitoneal injection of anti-CD8a antibody (BioXCell, clone 2.43, catalog #BP0061) (100 μg/mouse) on days 4 and 7 post tumor cell inoculation. When allograft tumors on both sides of the mice grew to about 150 mm³ in size, mice were administered with saline, anti-CD25 antibody PC61-IR700 conjugate (100 μg), anti-PD-1 antibody RMP1-14 (100 μg), or a combination of PC61-IR700 conjugate (100 μg) and RMP1-14 (100 μg). The PC61-IR700 conjugate was administered at day 4 and RMP1-14 at days 4, 6, 8, and 11. Twenty-four hours after administration of PC61-IR700 conjugate, tumors on the right flanks in the mice of PIT group were illuminated at 690 nm at a dose of 100 J/cm², while those on the left flanks were not illuminated. Thus, the right flank tumors served as primary tumors, and the left flank tumors served as distal tumors.

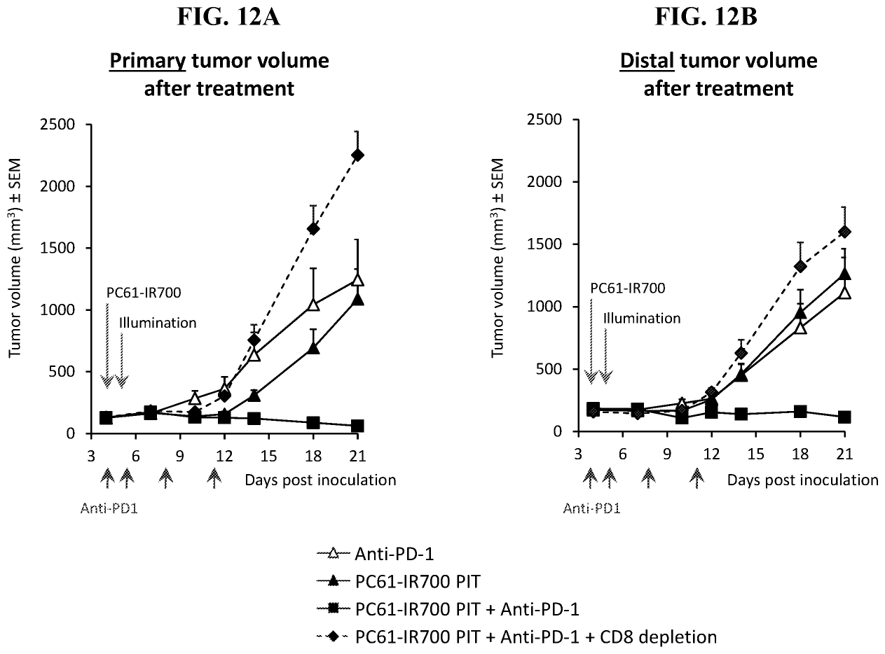

As shown in FIGS. 12A-12B, combination treatment of PC61-IR700 conjugate with illumination (PC61-IR700 PIT) and anti-PD-1 (RMP1-14) substantially and synergistically inhibited tumor growth of both illuminated tumors (right flank; FIG. 12A) and non-illuminated distal tumors (left flank; FIG. 12B) in immunocompetent BALB/c mice (closed squares) compared to the individual PC61-IR700 PIT (closed triangles) or anti-PD-1 (open triangles) monotherapies. The growth of both illuminated tumors (right flank) and non-illuminated distal tumors (left flank) was substantially inhibited in comparison to the control saline or PC61-IR700 conjugate only (non-illuminated) groups (data not shown). Surprisingly, in mice depleted of CD8⁺ T cells, the inhibitory effect of aa combination of PC61-IR700 PIT and anti-PD-1 on tumor growth was completely abolished (FIGS. 12A and 12B; closed diamonds), indicating the effect of the combination treatment was mediated by CD8⁺ T cells.

### Example 14 Reduction of Immunosuppressive Intratumoral Regulatory T Cell (Treg) by CD25 PIT and Anti-PD-1 Treatment

This example describes the effect of an exemplary anti-CD25 conjugate PIT treatment in combination with an anti-PD-1 antibody on the intratumoral CD8 T cell to Treg ratio.

BALB/c mice were inoculated with 1×10⁶ CT26-EphA2 clone c4D10 cells subcutaneously on the right hind flank. When allograft tumors grew to about 150 mm³ in size, mice were administered a saline control, anti-CD25 antibody PC61-IR700 conjugate (100 μg), or a combination of PC61-IR700 conjugate (100 μg) and an anti-PD-1 antibody (clone RMP1-14, 100 μg). The PC61-IR700 conjugate was administered at Day 7 and the anti-PD-1 antibody was administered at Days 7, 9, 11, and 13. Twenty-four hours after administration of the PC61-IR700 conjugate, tumors on the right flanks in the mice of PIT group were illuminated at 690 nm at a dose of 100 J/cm².

At eight days post illumination, tumors were excised from a first subset and second subset of mice, respectively from each of the treatment groups. The excised tumors were processed into single cell suspensions. Suspended cells were then stained for cell markers including CD3, CD45, CD4, and FoxP3. Isotype controls were also used for staining. The stained cells were analyzed using flow cytometry.

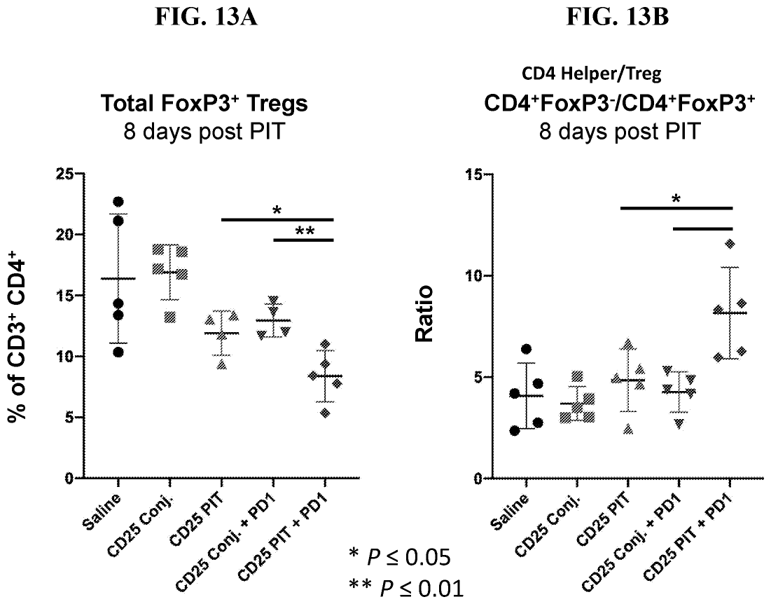

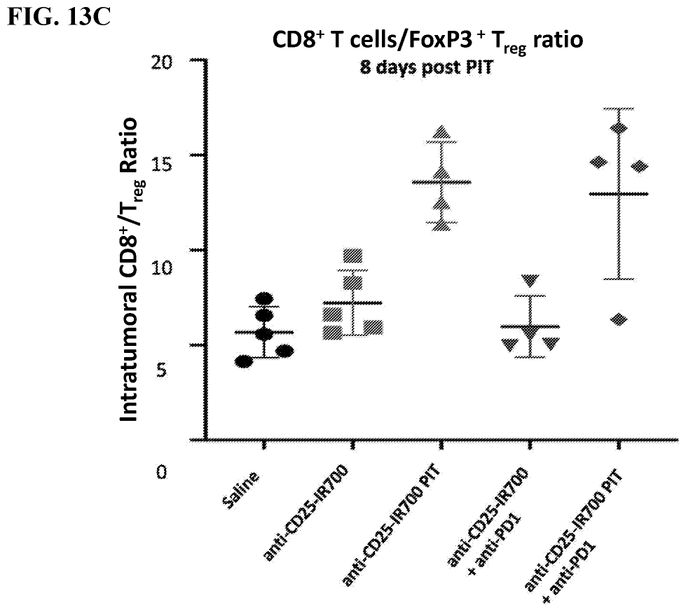

Flow cytometry analysis demonstrated that the combination of the anti-CD25 conjugate PIT treatment and the anti-PD-1 antibody (CD25 PIT+PD1) provided a durable reduction in the FoxP3⁺ Tregs (FIG. 13A). The decrease in FoxP3⁺ Tregs with the combination treatment was significant compared to the combination treatment of the conjugate and anti-PD-1 antibody without illumination (CD25 Conj.+PD1) (P<0.01) and compared to the anti-CD25 conjugate PIT treatment alone without the anti-PD-1 antibody (CD25 PIT) (P<0.05)

The combination treatment (CD25 PIT+PD1) also provided a significant increase in the ratio of CD4⁺ helper T cells (CD4⁺FoxP3⁻) to Treg cells (CD4⁺FoxP3⁺) (FIG. 13B). This increase was significant compared to the combination of the conjugate and anti-PD-1 antibody without illumination (CD25 Conj.+PD1) (P≤0.05) and compared to the anti-CD25 conjugate PIT treatment alone without anti-PD-1 antibody (CD25 PIT) (P<0.05). The effect of the anti-CD25 conjugate PIT with the anti-PD-1 antibody provides a synergistic enhancement of the intratumoral CD4⁺ to Treg ratio.

The anti-CD25 conjugate PIT treatment alone (CD25 PIT) or in combination with the anti-PD-1 antibody (CD25 PIT+PD1) resulted in a substantial increase in the intratumoral CD8 T cells to FoxP3 Treg ratio compared to control groups (FIG. 13C).

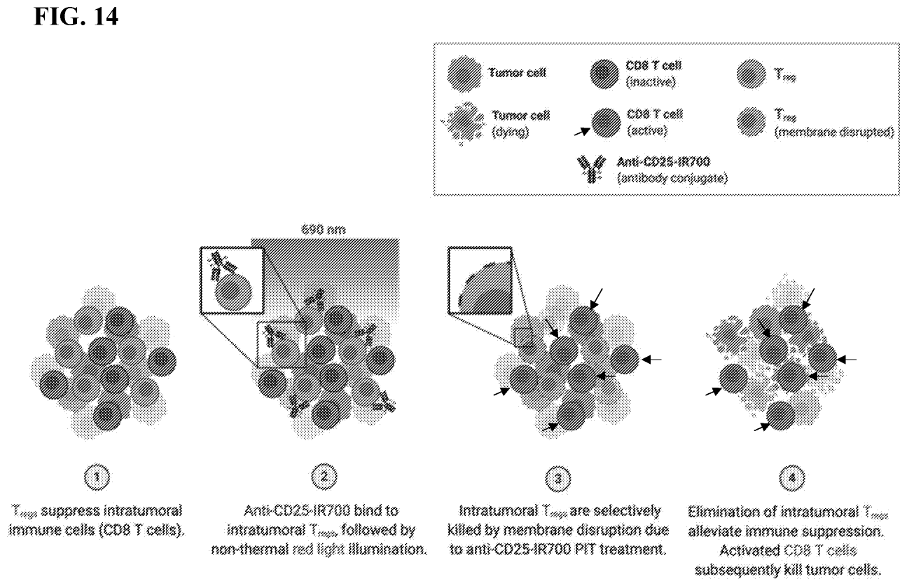

These results indicate that the combination treatment of anti-CD25 conjugate PIT with an anti-PD-1 antibody treatment elicits a durable reduction of intratumoral Tregs. A proposed mechanism of action is depicted in FIG. 14.

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. A method for treating a cancer, comprising:
(a) administering an anti-PD-1 antibody to a subject having a cancer comprising a first tumor and a secondary population of tumor cells;
(b) administering a conjugate comprising a phthalocyanine dye linked to an anti-CD25 antibody to the subject; and
(c) after administering the conjugate, illuminating the first tumor at a wavelength of at or about 600 nm to at or about 850 nm and at a dose of from at or about 25 J/cm² to at or about 400 J/cm² or from at or about 2 J/cm fiber length to at or about 500 J/cm fiber length, wherein the secondary population is not directly illuminated;
wherein the first tumor and/or the secondary population is inhibited to a greater degree as compared with administration of only the conjugate, only the conjugate followed by illumination, or only the anti-PD-1 antibody.

2. The method of claim 1, wherein inhibition comprises one or more of: less than at or about 20% increase in tumor volume, tumor dimensions or tumor mass, or a reduction in tumor volume, tumor dimensions or tumor mass, or a reduction in the number of tumor cells.

3. The method of claim 2, wherein the reduction in tumor volume, tumor dimensions or tumor mass, or the number of tumor cells comprises at or about a 30% reduction or more.

4. The method of any of claims 1-3, wherein the secondary population comprises metastatic tumor cells.

5. The method of any of claims 1-3, wherein the secondary population comprises invasive tumor cells.

6. The method of any of claims 1-5, wherein the secondary population comprises metastatic tumor cells and invasive tumor cells.

7. The method of any of claims 1-6, wherein the anti-PD-1 antibody is administered to the subject concurrently with the conjugate.

8. The method of any of claims 1-7, wherein the anti-PD-1 antibody is administered to the subject within 24 to 48 hours of administering the conjugate.

9. The method of any of claims 1-8, wherein the anti-PD-1 antibody is administered to the subject within 24 hours±4 hours of administering the conjugate.

10. The method of any of claims 1-9, wherein a first dose of the anti PD-1 antibody is administered prior to the administration of the conjugate.

11. The method of claim 10, wherein the first dose of the anti PD-1 antibody is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

12. The method of any of claims 1-11, wherein the anti-PD-1 antibody is administered once, twice, three times, four times, five times, six times, seven times, eight times, nine times, 10 times, or more than 10 times to the subject.

13. The method of any of claims 1-12, wherein anti-PD-1 antibody is administered once, twice, three times, four times, five times, six times, seven times, eight times, nine times, or 10 times before the administration of the conjugate, and subsequently followed by the administration of the anti-PD-1 antibody zero times, once, twice, three times, four times, five times, six times, seven times, eight times, nine times, 10 times, or more than 10 times to the subject after administration of the conjugate.

14. The method of any of claims 1-12, wherein the anti-PD-1 antibody is administered concurrently with the administration of the conjugate, and subsequently followed by the administration of the anti-PD-1 antibody zero times, once, twice, twice, three times, four times, five times, six times, seven times, eight times, nine times, 10 times, or more than 10 times to the subject after administration of the conjugate.

15. The method of any of claims 1-12, wherein the anti-PD-1 antibody is administered once, twice, three times, four times, five times, six times, seven times, eight times, nine times, 10 times, or more than 10 times after the administration of the conjugate to the subject.

16. The method of any of claims 1-15, wherein the secondary population is comprised in a solid tumor.

17. The method of any of claims 1-16, wherein the subject exhibits a complete response.

18. The method of any of claims 1-17, wherein the anti-CD25 antibody comprises a functional Fc region.

19. The method of any of claims 1-17, wherein the anti-CD25 antibody is basiliximab or daclizumab, or biosimilar, interchangeable, biobetter, copy biological or biogeneric thereof.

20. The method of claim 19, wherein the anti-CD25 antibody is basiliximab.

21. The method of any of claims 1-20, wherein the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

22. The method of any of claims 1-21, wherein the phthalocyanine dye is a Si-phthalocyanine dye.

23. The method of claim 22, wherein the Si-phthalocyanine dye is IR700.

24. The method of any of claims 1-23, wherein the illumination is carried out between 30 minutes and 96 hours after administering the conjugate.

25. The method of claim 24, wherein the illumination is carried out 24 hours±4 hours after administering the conjugate.

26. The method of any of claims 1-25, wherein the first tumor is illuminated at a wavelength of 690±40 nm.

27. The method of any of claims 1-26, wherein the first tumor is illuminated at a dose of or about of 50 J/cm² or 100 J/cm of fiber length.

28. The method of any of claims 1-27, further comprising (d) administering an additional therapeutic agent or anti-cancer treatment.

29. The method of any of claims 1-28, wherein one or more of steps (a), (b), (c), or (d) are repeated.

30. The method of any of claims 1-29, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

31. The method of any of claims 1-30, wherein the second population is located in one, two, three, or more than three tissues or organs that are different from the tissue or organ where the first tumor is located.

32. The method of any of claims 1-31, wherein the first tumor and/or the secondary population is inhibited to a greater degree as compared with administration of only the conjugate followed by illumination, and as compared with administration of only the anti-PD-1 antibody.

33. A method for generating an enhanced response in a subject having a cancer, comprising:
(a) administering an anti-PD-1 antibody to the subject having a cancer comprising a tumor;
(b) administering a conjugate comprising a phthalocyanine dye linked to an anti-CD25 antibody to the subject, wherein the anti-PD-1 antibody is administered prior to or concurrently with the conjugate; and
(c) after administering the conjugate, illuminating the tumor at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein:
the enhanced response comprises an enhancement of systemic immunity of the subject as compared to the systemic immunity of the subject prior to the administration of the conjugate followed by illumination and the anti-PD-1 antibody; and/or
the enhanced response comprises an enhancement of inhibition of the tumor as compared with administration of only the conjugate, only the conjugate followed by illumination, or only the anti-PD-1 antibody.

34. The method of claim 33, wherein the enhanced response is additive or synergistic.

35. The method of claim 33 or 34, wherein inhibition comprises one or more of: less than at or about 20% increase in tumor volume, tumor dimensions or tumor mass, or a reduction in tumor volume, tumor dimensions or tumor mass, or a reduction in the number of tumor cells.

36. The method of claim 35, wherein the reduction in tumor volume, tumor dimensions or tumor mass, or the number of tumor cells comprises at or about a 30% reduction or more.

37. The method of any of claims 33-36, wherein:
the tumor comprises a first tumor and a secondary population of tumor cells, and wherein the first tumor is illuminated and the secondary population is not directly illuminated;
the tumor comprises a first tumor and metastatic tumor cells, and wherein the first tumor is illuminated and the metastatic tumor cells are not directly illuminated; and/or
the tumor comprises a first tumor and invasive tumor cells, and wherein the first tumor is illuminated and the invasive tumor cells are not directly illuminated.

38. The method of any of claims 34-37, wherein the enhanced response is a synergistic response, wherein the synergistic response comprises a synergistic reduction in the growth, tumor volume, tumor dimensions or tumor mass of a first tumor, a synergistic reduction in the number of cells in the secondary population in the subject, a synergistic reduction in the growth, tumor volume, tumor dimensions, tumor mass, or the number of metastatic or invasive tumor cells, or any combination thereof.

39. The method of any of claims 33-38, wherein the anti-PD-1 antibody is administered to the subject within 24 hour to 48 hours of administering the conjugate.

40. The method of any of claims 33-39, wherein the anti-PD-1 antibody is administered to the subject within 24 hours±4 hours of administering the conjugate.

41. The method of any of claims 33-40, wherein a first dose of the anti PD-1 antibody is administered prior to the administration of the conjugate.

42. The method of claim 41, wherein the first dose of the anti PD-1 antibody is administered at least 1 day, 2 days, 3 days, 4 days, 5 days, 6 days, 7 days, 8 days, 9, days, 10 days, 11 days, 12 days, 13 days, 14 days, 1 week, 2 weeks, 3 weeks, 4 weeks, 5 weeks, 6 weeks 7 weeks, 8 weeks, 9 weeks, or 10 weeks prior to the administration of the conjugate.

43. The method of any of claims 33-42, wherein systemic immunity is measured by one or more of a cytotoxic T lymphocyte (CTL) activity assay, an intratumoral T cell exhaustion assay, an intratumoral effector T cell expansion assay, a T cell receptor diversity assay, an activated CD8⁺ T cell assay, a circulating regulatory T cell (Treg) assay, an intratumoral Treg assay, or a CD8⁺ Tcell:Treg assay.

44. The method of any of claims 33-43, wherein systemic immunity is measured by a CTL activity assay using splenocytes or peripheral blood cells or bone marrow cells or lymph node cells, optionally collected from the subject between day 4 and day 28 after illumination of the first tumor in the subject.

45. The method of any of claims 33-43, wherein systemic immunity is measured by an intratumoral T cell exhaustion assay using T cells collected from the first tumor or a metastatic tumor cell mass or an invasive tumor cell mass, optionally collected from the subject between day 4 and day 28 after illumination of the first tumor in the subject.

46. The method of any of claims 33-43, wherein systemic immunity is measured by an intratumoral effector T cell expansion assay using T cells collected from the first tumor or a metastatic tumor cell mass or an invasive tumor cell mass, optionally collected from the subject between day 4 and day 28 after illumination of the first tumor in the subject.

47. The method of any of claims 33-43, wherein systemic immunity is measured by a T cell receptor diversity assay using T cells collected from the first tumor or a metastatic tumor cell mass or an invasive tumor cell mass or the peripheral circulation, optionally collected from the subject between day 4 and day 28 after illumination of the first tumor in the subject.

48. The method of any of claims 33-43, wherein systemic immunity is measured by determining the presence, number or frequency of regulatory T cells (Tregs) in the tumor and/or the ratio of intratumoral Treg cells to intratumoral CD8+ T cells or intratumoral CD4+ T cells from the first tumor or a metastatic tumor cell mass or an invasive tumor cell mass, optionally collected from the subject between day 4 and day 28 after illumination of the first tumor in the subject.

49. The method of any of claims 33-48, wherein the anti-CD25 antibody comprises a functional Fc region.

50. The method of any of claims 33-49, wherein the anti-CD25 antibody is basiliximab or daclizumab, or biosimilar, interchangeable, biobetter, copy biological or biogeneric thereof.

51. The method of claim 50, wherein the anti-CD25 antibody is basiliximab.

52. The method of any of claims 33-51, wherein the anti-PD-1 antibody is selected from the group consisting of pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, RO7121661, CX-188, spartalizumab, BCD-217, HX009, IBI308, PDR001, REGN2810, and TSR-042 (ANB011).

53. The method of any of claims 33-52, wherein the anti-PD-1 antibody is administered once, twice, three times, four times, five times, six times, seven times, eight times, nine times, 10 times, or more than 10 times to the subject.

54. The method of any of claims 33-53, wherein the phthalocyanine dye is a Si-phthalocyanine dye.

55. The method of claim 54, wherein the Si-phthalocyanine dye is IR700.

56. The method of any of claims 33-55, wherein the illumination is carried out between 30 minutes and 96 hours after administering the conjugate.

57. The method of claim 56, wherein the illumination is carried out 24 hours±4 hours after administering the conjugate.

58. The method of any of claims 33-57, wherein the tumor is illuminated at a wavelength of 690±40 nm.

59. The method of any of claims 33-58, wherein the tumor is illuminated at a dose of or about of 50 J/cm² or 100 J/cm of fiber length.

60. The method of any of claims 33-59, further comprising (d) administering an additional therapeutic agent or anti-cancer treatment.

61. The method of any of claims 33-60, wherein one or more of steps (a), (b), (c), or (d) are repeated.

62. The method of any of claims 33-61, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

63. The method of any of claims 33-62, wherein the enhanced response comprises an additive response or a synergistic response as compared with administration of only the conjugate followed by illumination, and as compared with administration of only the anti-PD-1 antibody.
